(19) United States
(12) Patent Application Publication
(10) Pub. No.: US 2018/0333389 A1
(43) Pub. Date: Nov. 22, 2018

(54) VITAMIN E COMPOSITIONS AND METHODS OF USE THEREFOR

(71) Applicant: BIOELECTRON TECHNOLOGY CORPORATION, Mountain View, CA (US)

(72) Inventor: Guy M. Miller, Monte Sereno, CA (US)

(21) Appl. No.: 15/953,448

(22) Filed: Apr. 14, 2018

Related U.S. Application Data
(60) Provisional application No. 62/488,643, filed on Apr. 21, 2017.
(60) Provisional application No. 62/485,739, filed on Apr. 14, 2017.

(51) Int. Cl. A61K 31/355; C07D 311/72

(52) U.S. Cl. CPC A61K 31/355; A61K 31/122; A61K 2300/00; C07D 311/72

(57) ABSTRACT

Compositions comprising a synthetic, non-plant based, substantially pure stereoisomer or stereoisomers of an alpha, beta, gamma, or delta tocopherol or alpha, beta, gamma, or delta tocotrienol, or the quinone or hydroquinone form thereof, or a metabolite thereof, are disclosed. The compositions of the present disclosure can, for example, have improved activity in cells as compared to compositions comprising equimolar mixtures of all stereoisomers.

### CROSS REFERENCE TO RELATED APPLICATION

This application claims the benefit under 35 U.S.C. § 119 of U.S. provisional application No. 62/485,739, filed Apr. 14, 2017, and U.S. provisional application No. 62/488,643, filed Apr. 21, 2017, the contents of which are hereby incorporated by reference in their entirety.

### BACKGROUND

Vitamin E has been characterized as an important fat soluble anti-oxidant that interrupts the propagation of reactive oxygen species that spread through biological membranes or through fat, when its lipid content undergoes oxidation by reacting with more reactive lipid species to form more stable products. Though collated as a single vitamin, Vitamin E actually can be comprised of a number of different compounds, including alpha, beta, gamma, and delta tocopherols and tocotrienols. Multiple stereoisomers are possible for each compound, with the (R,R,R) form being the naturally occurring configuration for tocopherols, and the (R,E,E) form being the naturally occurring configuration for tocotrienols.

The harvesting and purification of Vitamin E from natural sources has long presented economic and environmental challenges. In particular, Vitamin E is typically harvested from plant sources, such as palm trees and soybeans. Costs associated with their production can be high, whether from the standpoint of monetary costs of securing natural sources, e.g., as components of plant oils, such as palm oils, soybean oils or the like, processing costs for extracting and purifying the Vitamin E components from complex plant oils, or in terms of environmental costs, as agricultural impacts on natural environments. In particular, some plant sources of Vitamin E, such as soybeans, are primarily available as GMOs, which may present regulatory constraints in certain countries. Furthermore, plant-sourced Vitamin E is often present in unspecified ratios of the various tocopherols and tocotrienols. One particular challenge with obtaining Vitamin E from plant sources is to obtain high quantities of a product that is at least modestly pure.

As a result of these costs, synthetic processes have been developed and proliferated for the production of Vitamin E, but a drawback of existing synthetic formulations is that they exist as racemic mixtures, or as equimolar mixtures of diastereomers (an equimolar mixture of the eight alpha tocopherol diastereomers is often referred to as “all racemic alpha tocopherol” or “all-rac alpha-tocopherol”).

### SUMMARY

In one aspect is provided a composition comprising a synthetic, non-plant based, substantially pure stereoisomer of an alpha, beta, gamma, or delta tocopherol or tocotrienol, or the quinone or hydroquinone form thereof, or a metabolite thereof. In some or any embodiments, provided is a composition wherein the stereoisomer is selected from the group consisting of: (R,R,R)-alpha-tocopherol, (R,R,R)-beta-tocopherol, (R,R,R)-gamma-tocopherol, (R,R,R)-delta-tocopherol, (R,E,E)-alpha-tocotrienol, (R,E,E)-beta-tocotrienol, (R,E,E)-gamma-tocotrienol, (R,E,E)-delta-tocotrienol, (R,R,R)-alpha-tocopherol quinone, (R,R,R)-beta-tocopherol quinone, (R,R,R)-gamma-tocopherol quinone, (R,R,R)-delta-tocopherol quinone, (R,E,E)-alpha-tocotrienol quinone, (R,E,E)-beta-tocotrienol quinone, (R,E,E)-gamma-tocotrienol quinone, (R,E,E)-delta-tocotrienol quinone, (R,R,R)-alpha-tocopherol hydroquinone, (R,R,R)-beta-tocopherol hydroquinone, (R,R,R)-gamma-tocopherol hydroquinone, (R,R,R)-delta-tocopherol hydroquinone, (R,E,E)-alpha-tocotrienol hydroquinone, (R,E,E)-beta-tocotrienol hydroquinone, (R,E,E)-gamma-tocotrienol hydroquinone, (R,E,E)-delta-tocotrienol hydroquinone, and metabolites thereof. In some or any embodiments, provided is the composition wherein the stereoisomer is (R,R,R)-alpha-tocopherol. In some or any embodiments, provided is a composition wherein the stereoisomer is (R,R,R)-beta-tocopherol. In some or any embodiments, provided is a composition wherein the stereoisomer is (R,R,R)-gamma-tocopherol. In some or any embodiments, provided is a composition wherein the stereoisomer is (R,R,R)-delta-tocopherol. In some or any embodiments, provided is a composition wherein the stereoisomer is (R,E,E)-alpha-tocotrienol. In some or any embodiments, provided is a composition wherein the stereoisomer is (R,E,E)-beta-tocotrienol. In some or any embodiments, provided is a composition wherein the stereoisomer is (R,E,E)-gamma-tocotrienol. In some or any embodiments, provided is a composition wherein the stereoisomer is (R,E,E)-delta-tocotrienol. In some or any embodiments, provided is a composition wherein the stereoisomer is (R,R,R)-alpha-tocopherol quinone. In some or any embodiments, provided is a composition wherein the stereoisomer is (R,R,R)-beta-tocopherol quinone. In some or any embodiments, provided is a composition wherein the stereoisomer is (R,R,R)-gamma-tocopherol quinone. In some or any embodiments, provided is a composition wherein the stereoisomer is (R,R,R)-delta-tocopherol quinone. In some or any embodiments, provided is a composition wherein the stereoisomer is (R,E,E)-alpha-tocotrienol quinone. In some or any embodiments, provided is a composition wherein the stereoisomer is (R,E,E)-beta-tocotrienol quinone. In some or any embodiments, provided is a composition wherein the stereoisomer is (R,E,E)-gamma-tocotrienol quinone. In some or any embodiments, provided is a composition wherein the stereoisomer is (R,E,E)-delta-tocotrienol quinone. In some or any embodiments, provided is a composition wherein the stereoisomer is (R,R,R)-alpha-tocopherol hydroquinone. In some or any embodiments, provided is a composition wherein the stereoisomer is (R,R,R)-beta-tocopherol hydroquinone. In some or any embodiments, provided is a composition wherein the stereoisomer is (R,R,R)-gamma-tocopherol hydroquinone. In some or any embodiments, provided is a composition wherein the stereoisomer is (R,R,R)-delta-tocopherol hydroquinone. In some or any embodiments, provided is a composition wherein the stereoisomer is (R,E,E)-alpha-tocotrienol hydroquinone. In some or any embodiments, provided is a composition wherein the stereoisomer is (R,E,E)-beta-tocotrienol hydroquinone. In some or any embodiments, provided is a composition wherein the stereoisomer is (R,E,E)-gamma-tocotrienol hydroquinone. In some or any embodiments, provided is a composition wherein the stereoisomer is (R,E,E)-delta-tocotrienol hydroquinone. In some or any embodiments, provided is a composition wherein the substantially pure stereoisomer comprises at least about 0.001% by weight of the composition. In some or any embodiments, provided is a composition wherein the substantially pure stereoisomer comprises at least about 0.01% by weight of the composition. In some or any embodiments, provided is a composition wherein the substantially pure stereoisomer comprises at least about 0.1% by weight of the composition. In some or any embodiments, provided is a composition wherein the substantially pure stereoisomer comprises at least about 1.0% by weight of the composition. In some or any embodiments, provided is a composition wherein the substantially pure stereoisomer comprises at least about 10.0% by weight of the composition. In some or any embodiments, provided is a composition wherein the purity is at least about 90%. In some or any embodiments, provided is a composition wherein the purity is at least about 95%. In some or any embodiments, provided is a composition wherein the purity is at least about 98%. In some or any embodiments, provided is a composition wherein the purity is at least about 99%. In some or any embodiments, provided is a composition wherein the purity is at least about 99.5%. In some or any embodiments, provided is a composition wherein the composition is a food, a functional food, a medical food, a pharmaceutical formulation, a nutritional supplement, a nutraceutical supplement, a vitamin supplement, a cosmetic formulation, a skin care formulation, or an ophthalmic formulation. In some or any embodiments, provided is a composition wherein the composition is formulated for human use. In some or any embodiments, provided is a composition wherein the composition is formulated for animal use. In some or any embodiments, provided is a composition wherein the composition is formulated for enteral, oral, parenteral, sublingual, inhalation, or rectal use. In some or any embodiments, provided is a composition wherein the composition is formulated for topical use. In some or any embodiments, provided is a composition wherein the composition is formulated for oral use. In some or any embodiments, a composition enriched in one or more of the desired stereoisomers is produced without chromatography techniques.

In another aspect, provided is a composition comprising one or more synthetic, non-plant based, substantially pure stereoisomers of: (a) a tocopherol selected from the group consisting of: alpha-tocopherol, beta-tocopherol, gamma-tocopherol, and delta-tocopherol, wherein the one or more stereoisomers of the tocopherol comprise an asymmetric carbon at the 2-position of the chromanol ring, wherein the 2-position asymmetric carbon has an R configuration; or a metabolite thereof; or (b) a tocopherol quinone selected from the group consisting of alpha-tocopherol quinone, beta-tocopherol quinone, gamma-tocopherol quinone, and delta-tocopherol quinone, wherein the one or more stereoisomers of the tocopherol quinone comprise an asymmetric carbon at the 3-position of the tail group, wherein the 3-position of the tail group has an R configuration; or a metabolite thereof; or (c) a tocopherol hydroquinone selected from the group consisting of alpha-tocopherol hydroquinone, beta-tocopherol hydroquinone, gamma-tocopherol hydroquinone, and delta-tocopherol hydroquinone, wherein the one or more stereoisomers of the tocopherol quinone comprise an asymmetric carbon at the 3-position of the tail group, wherein the 3-position of the tail group has an R configuration; or a metabolite thereof. In some or any embodiments, provided is a composition wherein the one or more stereoisomers are alpha-tocopherol stereoisomers. In some or any embodiments, provided is a composition wherein the one or more stereoisomers are beta-tocopherol stereoisomers. In some or any embodiments, provided is a composition wherein the one or more stereoisomers are gamma-tocopherol stereoisomers. In some or any embodiments, provided is a composition wherein the one or more stereoisomers are delta-tocopherol stereoisomers. In some or any embodiments, provided is a composition wherein the one or more stereoisomers are alpha-tocopherol quinone stereoisomers. In some or any embodiments, provided is a composition wherein the one or more stereoisomers are beta-tocopherol quinone stereoisomers. In some or any embodiments, provided is a composition wherein the one or more stereoisomers are gamma-tocopherol quinone stereoisomers. In some or any embodiments, provided is a composition wherein the one or more stereoisomers are delta-tocopherol quinone stereoisomers. In some or any embodiments, provided is a composition wherein the one or more stereoisomers are alpha-tocopherol hydroquinone stereoisomers. In some or any embodiments, provided is a composition wherein the one or more stereoisomers are beta-tocopherol hydroquinone stereoisomers. In some or any embodiments, provided is a composition wherein the one or more stereoisomers are gamma-tocopherol hydroquinone stereoisomers. In some or any embodiments, provided is a composition wherein the one or more stereoisomers are delta-tocopherol hydroquinone stereoisomers. In some or any embodiments, provided is a composition wherein the substantially pure stereoisomer or stereoisomers comprise at least about 0.001% by weight of the composition. In some or any embodiments, provided is a composition wherein the substantially pure stereoisomer or stereoisomers comprise at least about 0.01% by weight of the composition. In some or any embodiments, provided is a composition wherein the substantially pure stereoisomer or stereoisomers comprise at least about 0.1% by weight of the composition. In some or any embodiments, provided is a composition wherein the substantially pure stereoisomer or stereoisomers comprise at least about 1.0% by weight of the composition. In some or any embodiments, provided is a composition wherein the substantially pure stereoisomer or stereoisomers comprise at least about 10.0% by weight of the composition. In some or any embodiments, provided is a composition wherein the purity is at least about 90%. In some or any embodiments, provided is a composition wherein the purity is at least about 95%. In some or any embodiments, provided is a composition wherein the purity is at least about 98%. In some or any embodiments, provided is a composition wherein the purity is at least about 99%. In some or any embodiments, provided is a composition wherein the purity is at least about 99.5%. In some or any embodiments, provided is a composition wherein the composition is a food, a functional food, a medical food, a pharmaceutical formulation, a nutritional supplement, a nutraceutical supplement, a vitamin supplement, a cosmetic formulation, a skin care formulation, or an ophthalmic formulation. In some or any embodiments, provided is a composition wherein the composition is formulated for human use. In some or any embodiments, provided is a composition wherein the composition is formulated for animal use. In some or any embodiments, provided is a composition wherein the composition is formulated for enteral, oral, parenteral, sublingual, inhalation, or rectal use. In some or any embodiments, provided is a composition wherein the composition is formulated for topical use. In some or any embodiments, provided is a composition wherein the composition is formulated for oral use. In some or any embodiments, a composition enriched in one or more of the desired stereoisomers is produced without chromatography techniques.

In another aspect, provided is a composition comprising one or more synthetic, non-plant based stereoisomers of a compound selected from the group consisting of alpha, beta, gamma, and delta tocopherol or tocotrienol, and the quinone and hydroquinone forms thereof, wherein the synthetic, non-plant based stereoisomers have an activity that is at least about 5% greater than an equimolar mixture of all diastereomers of the compound. In some or any embodiments, provided is a composition wherein the composition is a food, a functional food, a medical food, a pharmaceutical formulation, a nutritional supplement, a nutraceutical supplement, a vitamin supplement, a cosmetic formulation, a skin care formulation, or an ophthalmic formulation. In some or any embodiments, provided is a composition wherein the composition is formulated for human use. In some or any embodiments, provided is a composition wherein the composition is formulated for animal use. In some or any embodiments, provided is a composition wherein the composition is formulated for enteral, oral, parenteral, sublingual, inhalation, or rectal use. In some or any embodiments, provided is a composition wherein the composition is formulated for topical use. In some or any embodiments, provided is a composition wherein the composition is formulated for oral use. In some or any embodiments, a composition enriched in one or more of the desired stereoisomers is produced without chromatography techniques.

In another aspect, provided is a composition comprising one or more synthetic, non-plant based stereoisomers of a compound selected from the group consisting of alpha, beta, gamma, and delta tocopherol or tocotrienol, and the quinone and hydroquinone forms thereof; wherein the one or more synthetic, non-plant based stereoisomers of the tocopherol or tocotrienol comprise an asymmetric carbon at the 2-position of the chromanol ring, wherein the 2-position asymmetric carbon has an R configuration; wherein the one or more synthetic, non-plant based stereoisomers of the tocopherol or tocotrienol quinone or hydroquinone comprise an asymmetric carbon at the 3-position of the tail group, wherein the 3-position of the tail group has an R configuration; and wherein the synthetic, non-plant based stereoisomers have an activity that is at least about 5% greater than an equimolar mixture of the corresponding R and S isomers of the compound. In some or any embodiments, provided is a composition wherein the composition is a food, a functional food, a medical food, a pharmaceutical formulation, a nutritional supplement, a nutraceutical supplement, a vitamin supplement, a cosmetic formulation, a skin care formulation, or an ophthalmic formulation. In some or any embodiments, provided is a composition wherein the composition is formulated for human use. In some or any embodiments, provided is a composition wherein the composition is formulated for animal use. In some or any embodiments, provided is a composition wherein the composition is formulated for enteral, oral, parenteral, sublingual, inhalation, or rectal use. In some or any embodiments, provided is a composition wherein the composition is formulated for topical use. In some or any embodiments, provided is a composition wherein the composition is formulated for oral use. In some or any embodiments, a composition enriched in one or more of the desired stereoisomers is produced without chromatography techniques.

In another aspect, provided is a composition comprising a synthetic, non-plant based alpha-tocopherol, wherein the composition has an alpha-tocopherol activity that is equal to or greater than about 1.1 IU per mg of synthetic, non-plant based alpha-tocopherol. In some or any embodiments, provided is a composition wherein the activity is equal to or greater than about 1.4 IU per mg of synthetic, non-plant based alpha-tocopherol. In some or any embodiments, provided is a composition wherein the synthetic, non-plant based alpha-tocopherol comprises a stereoisomer of alpha-tocopherol, wherein the stereoisomer comprises an asymmetric carbon at the 2-position of the chromanol ring, wherein the asymmetric carbon has an R configuration. In some or any embodiments, provided is a composition wherein the stereoisomer comprises three asymmetric carbons, wherein each asymmetric carbon has an R configuration. In some or any embodiments, provided is a composition wherein the stereoisomer is substantially pure. In some or any embodiments, provided is a composition wherein the composition is formulated for human use. In some or any embodiments, provided is a composition wherein the composition is formulated for animal use. In some or any embodiments, provided is a composition wherein the composition is formulated for enteral, oral, parenteral, sublingual, inhalation, or rectal use. In some or any embodiments, provided is a composition wherein the composition is formulated for topical use. In some or any embodiments, provided is a composition wherein the composition is formulated for oral use. In some or any embodiments, a composition enriched in one or more of the desired stereoisomers is produced without chromatography techniques.

In another aspect, provided is a composition comprising a synthetic, non-plant based alpha-tocopherol quinone, wherein the composition has a baseline activity in a Sulfide: quinone oxidoreductase colorimetric assay that is at least about 5% higher than a baseline activity of a composition that comprises a mixture of RRR- and SRR-alpha-tocopherol quinones with a diastereomeric ratio of about 1:1 in the assay, wherein each composition has an equivalent amount of RRR-alpha-tocopherol quinone. In some or any embodiments, provided is a composition wherein the composition is formulated for human use. In some or any embodiments, provided is a composition wherein the composition is formulated for animal use. In some or any embodiments, provided is a composition wherein the composition is formulated for enteral, oral, parenteral, sublingual, inhalation, or rectal use. In some or any embodiments, provided is a composition wherein the composition is formulated for topical use. In some or any embodiments, provided is a composition wherein the composition is formulated for oral use. In some or any embodiments, a composition enriched in one or more of the desired stereoisomers is produced without chromatography techniques.

In another aspect, provided is a method of treating an individual, comprising: administering to a subject in need thereof a physiologically effective amount or a therapeutically effective amount of a composition any embodiments described herein, including any of the foregoing embodiments.

In some embodiments, the composition has an alpha-tocopherol activity that is equal to or greater than about 1.2 IU per mg of synthetic, non-plant based alpha-tocopherol. In some embodiments, the composition has an alpha-tocopherol activity that is equal to or greater than about 1.3 IU per mg of synthetic, non-plant based alpha-tocopherol. In some embodiments, the composition has an alpha-tocopherol activity that is equal to or greater than about 1.4 IU per mg of synthetic, non-plant based alpha-tocopherol. In some embodiments, the composition has an alpha-tocopherol activity that is equal to or greater than about 1.45 IU per mg of synthetic, non-plant based alpha-tocopherol. In some embodiments, the composition has an alpha-tocopherol activity that is equal to or greater than about 1.49 IU per mg of synthetic, non-plant based alpha-tocopherol. In some or any embodiments, a composition enriched in one or more of the desired stereoisomers is produced without chromatography techniques.

In some embodiments, the stereoisomer(s) have a C¹²/C¹³ molar ratio of less than about 95:1, less than about 90:1, less than about 80:1, less than about 70:1, less than about 60:1, or less than about 50:1. In some embodiments, a non-plant based compound of the present disclosure has a C¹²/C¹³ molar ratio of greater than about 99:1. In some embodiments, a non-plant based compound of the present disclosure has a C¹²/C¹³ molar ratio of greater than about 95:1, about 90:1, about 80:1, about 70:1, about 60:1, or about 50:1.

Additional aspects and advantages of the present disclosure will become readily apparent to those skilled in this art from the following detailed description, wherein only illustrative aspects of the present disclosure are shown and described. As will be realized, the present disclosure is capable of other and different aspects, and its several details are capable of modifications in various respects, all without departing from the present disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative aspects, in which the principles of the present disclosure are utilized, along with the accompanying drawings of which:

FIG. 1A illustrates SQR activity in the presence of R,R,R-alpha tocopherol quinone, S,R,R-alpha tocopherol quinone, and an equimolar mixture of R/S,R,R-alpha tocopherol quinone (“Ambo-ATQ”);

FIG. 1B illustrates SQR activity in the presence of increasing concentrations of R,R,R-alpha tocopherol quinone, with and without S,R,R-alpha tocopherol quinone;

FIG. 2 illustrates the amount of conversion of R,R,R-alpha-tocopherol (black bars) and all-rac-alpha tocopherol (grey bars) to the corresponding quinones in HepG2 cells;

FIG. 3 illustrates the amount of conversion of R,R,R-alpha-tocopherol (black bars) and all-rac-alpha tocopherol (grey bars) to the corresponding quinones in NHF cells; and

FIG. 4 illustrates the amount of conversion of R,R,R-alpha-tocopherol and all-rac-alpha tocopherol (grey bars) to the corresponding quinones in Q7 cells.

### DETAILED DESCRIPTION

The use of certain synthetic forms of Vitamin E for nutritional and other uses, typically present as “all-racemic” alpha-tocopherol (an equimolar mixture of all 8 stereoisomers of alpha tocopherol) has proliferated. However, the inventors have discovered that certain stereoisomeric forms of the Vitamin E constituents are not only less effective, but can actually operate as inhibitors to important biological enzyme systems. Moreover, certain metabolites of Vitamin E components have been demonstrated to provide improved characteristics over Vitamin E. The present disclosure addresses these differences by providing compositions that comprise selected stereoisomers of Vitamin E components and/or metabolites thereof, to address these challenges.

SQR, or sulfide:quinone oxidoreductase, is a mitochondrially-localized enzyme that, in humans, is encoded by the sqrdl gene. The physiological role of SQR is to clear hydrogen sulfide generated via multiple biological transformations. While hydrogen sulfide can exert toxic effects at relatively modest concentrations, at lower concentrations sulfide plays a role in modulating various cellular processes. To accomplish this, SQR catalyzes the two-electron oxidation of hydrogen sulfide to a sulfane sulfur (persulfide equivalent) with concomitant reduction of a quinone substrate to a hydroquinone. The endogenous quinone substrate in humans is believed to be coenzyme Q10. The inventors have discovered quinones derived from Vitamin E can also serve as oxidizing substrates for SQR. Without wishing to be bound by any particular theory, the inventors believe that in this capacity, the quinone metabolites of Vitamin E modulate the biological role of SQR in maintaining the appropriate hydrogen sulfide levels within a cell.

While various embodiments of this disclosure have been shown and described herein, it will be apparent to those skilled in the art that such embodiments are provided by way of example only. Numerous variations, changes, and substitutions may occur to those skilled in the art without departing from the inventive subject matter. It should be understood that various alternatives to the embodiments described herein may be employed.

The term “synthetic,” as used herein, refers to compounds that have been made by chemical synthesis in a man-made setting, i.e., compounds that are prepared by reaction of other compounds, such as through the chemical conversion or chemical modification of one or more precursor compounds, in a man-made setting. Such synthetic compounds exclude compounds that are isolated or purified from a natural source or from an organism. In some or any embodiments, none of the starting materials nor intermediates used in Examples 1-8 are isolated or purified from a natural source or from an organism.

The term “non-plant based,” as used herein, refers to a compound that is not isolated from a plant source and is not produced from a tocopherol or tocotrienol that has been isolated from a plant source. For example, a “non-plant based alpha tocopherol” excludes alpha tocopherol that has been synthesized by methylating beta-, gamma-, and/or delta-tocopherols that were isolated from a plant source. It further excludes alpha-tocopherol that was produced by hydrogenation of alpha-tocotrienol that was isolated from a plant source. Because plants have a preference for C¹² over C¹³, a compound that is produced by a plant will typically have a higher C¹²/C¹³ ratio than a compound that is not produced by a plant. In some embodiments, a non-plant based compound of the present disclosure has a C¹²/C¹³ molar ratio of less than 99:1. In some embodiments, a non-plant based compound of the present disclosure has a C¹²/C¹³ molar ratio of less than 95:1, 90:1, 80:1, 70:1, 60:1, or 50:1. In some embodiments, a non-plant based compound of the present disclosure has a C¹²/C¹³ molar ratio of greater than 99:1. In some embodiments, a non-plant based compound of the present disclosure has a C¹²/C¹³ molar ratio of greater than 95:1, 90:1, 80:1, 70:1, 60:1, or 50:1.

The term “alpha-tocopherol activity” indicates the activity in international units (IU). The different stereoisomers have different activity levels, as based on fertility enhancement by the prevention of miscarriages in pregnant rats. 1 IU is defined as 0.667 mg of RRR-alpha tocopherol.

The term “asymmetric carbon,” “chiral carbon,” or “stereocenter,” as used in reference to a carbon in a compound described herein, generally refers to a carbon atom that is attached to four different types of atoms or groups of atoms. A chiral carbon does not have a plane of symmetry, and thus, a compound that contains a chiral carbon does not have a plane of symmetry. An asymmetric carbon, chiral carbon, or stereocenter may be designated as having an R configuration or an S configuration, according to the (R)/(S) notation or the Cahn-Ingold Prelog Rules. In some embodiments, a compound may have an R stereocenter, an S stereocenter, or a combination of R and S stereocenters. In some embodiments, a compound that has a stereocenter may rotate plane polarized light.

The term “racemic mixture,” as used herein, generally refers to a substantially equal mixture of enantiomers of a chiral molecule or compound. In certain embodiments, the racemic mixture is about 50:50.

The term “stereoisomer,” as used herein, generally refers to a single compound of all stereoisomeric compounds that have the same sequence of bonded atoms and differ only in the three-dimensional orientation of their atoms in space. Stereoisomers include chiral carbons, and cis-trans orientation of double bonds. For example, if a compound has three stereocenters, the compound has a possibility of eight diastereomers, or eight stereoisomers. As used herein, a stereoisomer generally refers to a single compound of the possible eight stereoisomers.

The term “enantiomer,” as used herein, generally refers to one of two stereoisomers that are mirror images of each other, and that are not superimposable on each other.

The term “enantiomeric ratio,” as used herein, generally refers to the ratio of one enantiomer to another enantiomer. For example, if a solution contains 70% of the R isomer and 30% of the S isomer, the enantiomeric ratio, or e.r., will be 7:3 (R:S). If a solution contains a precise racemic mixture, i.e. 50% of the R isomer and 50% of the S isomer, the enantiomeric ratio will be 1:1 (R:S).

The term “diastereomeric ratio,” as used herein, generally refers to the ratio of one diastereomer to one or more other diastereomers. For example, alpha-tocopherol has three stereocenters. Thus, when referenced herein, an equal mixture of all eight potential alpha-tocopherol stereoisomers having three chiral centers with configurations of -RRR, -RRS, -RSR, -RSS, -SSS, -SSR, -SRS, and -SRR, corresponds to a diastereomeric ratio of the mixture as about 1:1:1:1:1:1:1:1.

The compounds of the present disclosure may contain one or more asymmetric centers and/or one or more carbon-carbon double bonds, and may thus give rise to enantiomers, diastereomers, and other stereoisomeric forms, defined in terms of absolute stereochemistry, as (R)- or (S)-, or geometrically as (E) or (Z). The present disclosure is meant to include all such possible isomers, as well as their racemic and optically pure forms.

The term “subject,” as used herein, generally includes humans of any age group, e.g., a pediatric subject (e.g., infant, child, or adolescent) or adult subject (e.g., young adult, middle-aged adult, or senior adult) and/or other primates (e.g., cynomolgus monkeys or rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. The methods described herein can be useful in both human therapeutics and veterinary applications. In some embodiments, the subject is a mammal, and in some embodiments, the subject is human. In some embodiments, a composition of the present disclosure is administered to a subject in need thereof.

The terms “medical food” or “clinical food” in certain embodiments used herein, generally refers to an ingestible composition that includes an active ingredient, such as an alpha-tocopherol or other composition described herein, in addition to one or more of digestible fats, carbohydrates and proteins. Medical foods or clinical foods may be prescribed and/or administered to a subject to address a specific patient population or condition, such as, for example, a specific deficiency or deficiency syndrome.

The term “pharmaceutically acceptable excipient,” as used herein, generally includes, for example, solvents, dispersion media, coatings, antibacterial and antifungal agents, isotonic and absorption delaying agents. Except insofar as any conventional media or agent is incompatible with the active ingredient, its use in the therapeutic compositions of the present disclosure is contemplated. Supplementary active ingredients can also be incorporated into the compositions.

The term “treating” or “treatment” as used herein, may include preventing a disease-state from occurring in a mammal, inhibiting a disease state, and relieving the disease state. Treating also may include the amelioration of a symptom of a disease (e.g., lessen the pain or discomfort), wherein such amelioration may or may not be directly affecting the disease (e.g., cause, transmission, expression, etc.).

The term “therapeutically-effective amount,” as used herein, generally refers to that amount of compound that is sufficient to effect treatment, when administered to a subject in need of such treatment. The therapeutically-effective amount will vary depending upon the subject and disease condition being treated, the weight and age of the subject, the severity of the disease condition, the manner of administration and the like, which can readily be determined by one of ordinary skill in the art.

A “physiologically effective amount” of an active substance indicates an adequate amount of the active substances to have a significant, externally observable effect on the patient. Thus, such a physiologically effective amount affects one or more of the characteristics in the patient without the need for special equipment to determine the effect. For example, a physiologically effective amount of a compound of the present invention has a significant, externally observable effect on the behavior of the patient by reducing one or more of the symptoms of the condition to be treated. Accordingly, one can determine whether an adequate amount of the active substance has been administered by watching the patient and observing whether changes have occurred in the patient due to the active substance.

The term “pharmaceutically acceptable,” as used herein, generally refers to those properties and/or substances that are acceptable to the patient from a pharmacological/toxicological point of view and to the manufacturing pharmaceutical chemist from a physical/chemical point of view regarding composition, formulation, stability, patient acceptance, and bioavailability.

The phrases “substantially pure” and “purity,” as used herein, refer to compositions that are enriched with a specific stereoisomer or stereoisomers of a particular compound, relative to other stereoisomers of the compound, i.e. purity of the specific stereoisomer(s) is measured relative to the amount of the other stereoisomers of the compound. In some embodiments, a composition that is substantially pure as to a specific stereoisomer or stereoisomers of a particular compound may comprise other stereoisomers of the compound in an amount not to exceed about 10% by weight, about 10% by mol, or 10% by volume of the total amount of all stereoisomers of the compound that are present in the composition (“purity is at least about 90%”). For instance, where a composition is recited as comprising substantially pure RRR-alpha-tocopherol, it will be understood that the total amount of all other alpha-tocopherol stereoisomers (RRS, RSR, SRR, SSR, SRS, RSS, SSS) in the composition will be less than about 10% by weight, about 10% by mol, or 10% by volume of the total amount of alpha-tocopherol present in said composition. For example, where a composition is recited as comprising substantially pure alpha-tocopherol having an R configuration at the 2-position of the chromanol ring, it will be understood that the total amount of all other alpha-tocopherol stereoisomers (SRR, SSR, SRS, SSS) in the composition will be less than about 10% by weight, about 10% by mol, or 10% by volume of the total amount of alpha-tocopherol present in said composition. In some embodiments, a composition that is substantially pure as to a specific stereoisomer or stereoisomers of a particular compound may comprise other stereoisomers of the compound in an amount not to exceed about 5% by weight, 5% by mol, or 5% by volume of the total amount of all stereoisomers of the compound that are present in the composition (“purity is at least about 95%”). In some embodiments, a composition that is substantially pure as to a specific stereoisomer or stereoisomers of a particular compound may comprise other stereoisomers of the compound in an amount not to exceed about 4% by weight, 4% by mol, or 4% by volume of the total amount of all stereoisomers of the compound that are present in the composition (“purity is at least about 96%”). In some embodiments, a composition that is substantially pure as to a specific stereoisomer or stereoisomers of a particular compound may comprise other stereoisomers of the compound in an amount not to exceed about 3% by weight, 3% by mol, or 3% by volume of the total amount of all stereoisomers of the compound that are present in the composition (“purity is at least about 97%”). In some embodiments, a composition that is substantially pure as to a specific stereoisomer or stereoisomers of a particular compound may comprise other stereoisomers of the compound in an amount not to exceed about 2% by weight, 2% by mol, 2% by volume of the total amount of all stereoisomers of the compound that are present in the composition (“purity is at least about 98%”). In some embodiments, a composition that is substantially pure as to a specific stereoisomer or stereoisomers of a particular compound may comprise other stereoisomers of the compound in an amount not to exceed about 1% by weight, 1% by mol, or 1$ by volume of the total amount of all stereoisomers of the compound that are present in the composition (“purity is at least about 99%”). In some embodiments, a composition that is substantially pure as to a specific stereoisomer or stereoisomers of a particular compound may comprise other stereoisomers of the compound in an amount not to exceed about 0.5% by weight, 0.5% by mol, or 0.5% by volume of the total amount of all stereoisomers of the compound that are present in the composition (“purity is at least about 99.5%”). When determining whether a stereoisomer is “substantially pure,” only the amounts of the synthetic, non-plant based stereoisomers are considered. For example, when determining the purity of (R,R,R)-alpha-tocopherol in a food, only the amounts of the various stereoisomers of synthetic, non-plant based alpha-tocopherol are considered; alpha-tocopherol that is naturally present in the food is not used in the determination of purity. In some or any embodiments, the % purity is % by weight purity.

The term “substantially free,” as used herein, refers to compositions that have a lower amount of a specific stereoisomer or stereoisomers of a particular compound, relative to other stereoisomers of the compound. In some embodiments, a composition that is substantially free of a specific stereoisomer or stereoisomers of a particular compound comprises less than about 10% by weight, 10% by mol, or 10% by volume of that stereoisomer(s), relative to the total amount of all stereoisomers of the compound that are present in the composition (“substantially free is less than about 10%”). For instance, where a composition is recited as being substantially free of alpha-tocopherol having a S configuration at the 2-position of the chromanol ring, it will be understood that the total amount of the SRR, SSR, SRS, and SSS stereoisomers in the composition will be less than about 10% by weight, 10% by mol, or 10% by volume of the total amount of alpha-tocopherol present in said composition. In some embodiments, a composition that is substantially free of a specific stereoisomer or stereoisomers of a particular compound comprises less than about 5% by weight, 5% by mol, or 5% by volume of that stereoisomer(s), relative to the total amount of all stereoisomers of the compound that are present in the composition (“substantially free is less than about 5%”). In some embodiments, a composition that is substantially free of a specific stereoisomer or stereoisomers of a particular compound comprises less than about 4% by weight, 4% by mol, or 4% by volume of that stereoisomer(s), relative to the total amount of all stereoisomers of the compound that are present in the composition (“substantially free is less than about 4%”). In some embodiments, a composition that is substantially free of a specific stereoisomer or stereoisomers of a particular compound comprises less than about 3% by weight, 3% by mol, or 3% by volume of that stereoisomer(s), relative to the total amount of all stereoisomers of the compound that are present in the composition (“substantially free is less than about 3%”). In some embodiments, a composition that is substantially free of a specific stereoisomer or stereoisomers of a particular compound comprises less than about 2% by weight, 2% by mol, or 2% by volume of that stereoisomer(s), relative to the total amount of all stereoisomers of the compound that are present in the composition (“substantially free is less than about 2%”). In some embodiments, a composition that is substantially free of a specific stereoisomer or stereoisomers of a particular compound comprises less than about 1% by weight, 1% by mol, or 1% by volume of that stereoisomer(s), relative to the total amount of all stereoisomers of the compound that are present in the composition (“substantially free is less than about 1%”). In some embodiments, a composition that is substantially free of a specific stereoisomer or stereoisomers of a particular compound comprises less than about 0.1% by weight, 0.1% by mol, or 0.1% by volume of that stereoisomer(s), relative to the total amount of all stereoisomers of the compound that are present in the composition (“substantially free is less than about 0.1%”). When determining whether a composition is “substantially free” of a particular stereoisomer, only the amounts of the synthetic, non-plant based stereoisomers of the compound are considered.

A “metabolite” indicates a human or animal metabolite of the specified compound.

The term “unit dosage form” generally refers to physically discrete units suitable as unitary dosages for human subjects and other mammals, each unit containing a predetermined quantity of active material calculated to produce the desired therapeutic effect, in association with a suitable pharmaceutical excipient.

The term “about” when referring to a number or a numerical range means that the number or numerical range referred to may be an approximation within experimental variability (or within statistical experimental error), and thus the number or numerical range may vary from, for example, 1% to 10% of the stated number or numerical range. The term about shall also include the absolute stated number. For example, a composition stated as having less than about 10% of a given component also includes within its stated range a composition that has less than exactly 10% of the given component. In some or any embodiments, the number or numerical range may vary from, in some embodiments, 1%, 2%, 3%, 4%, 5%, 6%, 7%, 8%, 9%, or 10%.

In any embodiment described herein, unless otherwise specified, a % of a compound within a composition may be measured as a weight %, a molar %, or a volume %. In one embodiment, a % of a compound within a composition may be measured as a weight %. In another embodiment, a % of a compound within a composition may be measured as a molar %. In another embodiment, a % of a compound within a composition may be measured as a volume %.

### Compositions

Provided herein are compositions that are enriched in one or more synthetic, non-plant based stereoisomers of tocopherols, tocotrienols, their corresponding quinones and hydroquinones, and/or their metabolites. In some embodiments, the composition comprises one or more substantially pure synthetic, non-plant based stereoisomers of tocopherols, tocotrienols, their corresponding quinones and hydroquinones, and/or their metabolites. In some embodiments, the composition is enriched in a particular stereoisomer(s) that have better activity and/or less toxicity and/or less off-target effects than other stereoisomers of the same compound. In some embodiments, the composition has better activity, less toxicity, and/or less off-target effects than the corresponding composition that comprises an equimolar mixture of all stereoisomers. In some embodiments, the composition is substantially free of one or more stereoisomers having, for example, lower activity, higher toxicity, and/or off-target effects. In some embodiments, the composition is substantially free or one or more stereoisomers that inhibit the activity of other stereoisomers.

Because there has been no knowledge of the potentially negative effects of certain stereoisomers of vitamin E components and their metabolites, there has been no attempt to move away from the production and consumption of the synthetically produced all-racemic vitamin E components. On the contrary, uses of synthetic all-racemic vitamin E components in human and animal nutrition, skin care, and hosts of other applications has continued to expand. Furthermore, the importance of using these compositions may be even greater when applied to challenged populations, such as, for example, diseased subjects, critical care subjects, or subjects in otherwise compromised states.

Synthetic processes have largely been employed in the large scale manufacture of vitamin E components. Synthetically produced tocopherols and tocotrienols, however, are produced and commercialized as an equimolar mixture of all stereoisomers varying at each of the chiral centers. Thus, there exists an unmet need to synthetically produce compositions containing stereopure tocopherols and tocotrienols.

### Tocopherols and Tocotrienols

The term “Vitamin E” includes four different tocopherol and tocotrienol compounds: alpha, beta, gamma and delta, which identify the substitution pattern around the chromanol ring of the molecule. The tocotrienols differ from the tocopherols in the unsaturation of the attached hydrophobic side chain. Depending on the particular source, all eight tocopherol and tocotrienol molecules can be present in Vitamin E.

The tocopherols include in their structure three stereocenters, providing the potential for eight different isomers. Naturally occurring tocopherols are usually found as the R,R,R stereoisomer. Alpha-tocotrienol has a single chiral center, and two double bonds which can be present as the Z or E isomer. Thus alpha tocotrienol can exist as the (R,E,E), (R,E,Z), (R,Z,E), (R,Z,Z), (S,E,E), (S,E,Z), (S,Z,E), and (S,Z,Z) isomers. Naturally occurring tocotrienols generally exist as the R,E,E isomer.

Reference to atom numbering is made throughout the specification. In some instances, numbering refers to a certain position on a chroman or chromanol ring and in other instances to a certain position in a tail group. The following formulae show atom numbering for representative structures of compounds disclosed herein. In certain embodiments (R,R,R)—corresponds to (2R,4′R,8′R)—as would be appreciated by a person of skill in the art.

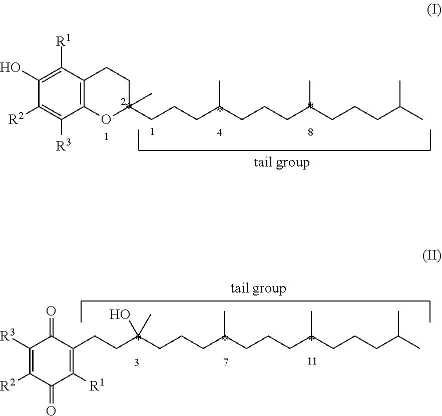

The following formulae show the substitution pattern on the chromanol or quinone core of the tocopherols and tocotrienols and their quinones, wherein an * symbol represents a chiral center. In some embodiments, when a structure does not explicitly describe stereochemistry at a chiral center, the lack of indication of stereochemistry indicates that this structure has both possible configurations (i.e. R- and S-) at the chiral center.

Formula (I) shows the structure of the alpha-, beta-, gamma-, and delta-tocopherols:

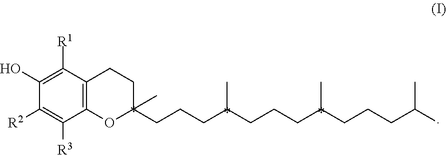

alpha tocopherol: R¹=Me, R²=Me, R³^{_{=Me;}}
beta tocopherol: R¹=Me R²^{^{H, R}}^{₃}=Me;
R₁^{=H, R}^{²}^{=Me, R}^{³}^{=Me;}
R₁^{H, R}^{²}H, R³=Me

Formula (II) shows the structure of the alpha-, beta-, gamma-, and delta-tocopherol quinones:

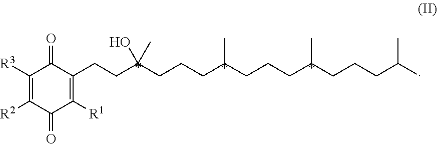

alpha tocopherol quinone: R¹^{^{=Me, R}}²=Me, R³=Me;
beta tocopherol quinone: R¹^{^{=Me, R}}²=H, R₃=Me;
gamma tocopherol quinone: R¹^{^{=H, R}}₂=Me, R₃=Me;
delta tocopherol quinone: R¹^{^{H, R}}₂=H, R³=Me

Formula (III) shows the structure of the alpha-, beta-, gamma-, and delta-tocotrienols (wherein the double bonds are present as the E isomer):

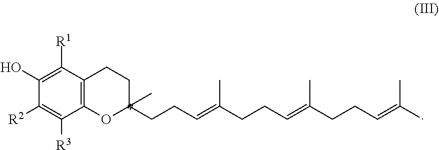

alpha tocotrienol: R¹^{^{=Me, R}}²=Me, R₃^{=Me;}
beta tocotrienol: R¹=Me, R₂^{=H, R}₃=Me;
gamma tocotrienol: R¹^{^{=H, R}}₂Me, R₃=Me;
delta tocotrienol: R¹^{^{=H, R}}^{₂}=H, R³=Me

Formula (IV) shows the structure of the alpha-, beta-, gamma-, and delta-tocotrienol quinones (wherein the double bonds are present as the E isomer):

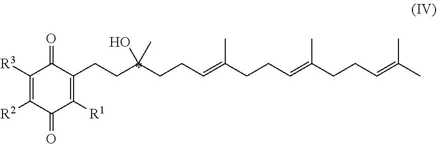

Alpha tocotrienol quinone: R¹=Me, R²=Me, R³=Me;
Beta tocotrienol quinone: R¹=Me, R²=H, R³=Me;
Gamma tocotrienol quinone: R¹=H, R²=Me, R³=Me;
Delta tocotrienol quinone: R¹=H, R²=H, R³=Me

Formula (V) shows the structure of the alpha-, beta-, gamma-, and delta-tocopherol hydroquinones:

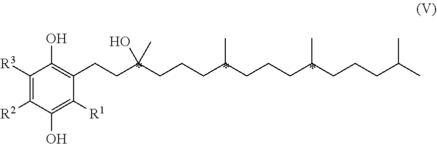

Alpha tocopherol hydroquinone: R¹=Me, R²=Me, R³=Me;
Beta tocopherol hydroquinone: R¹=Me, R²=H, R³=Me;
Gamma tocopherol hydroquinone: R¹=H, R²=Me, R³=Me;
Delta tocopherol hydroquinone: R¹=H, R²=H, R³=Me;

Formula (VI) shows the structure of the alpha-, beta-, gamma-, and delta-tocotrienol hydroquinones (wherein the double bonds are present as the E isomer):

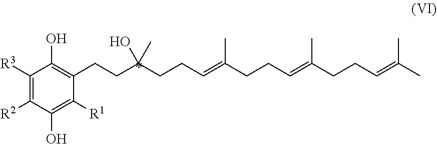

Alpha tocopherol hydroquinone: R¹=Me, R²=Me, R³=Me;
Beta tocopherol hydroquinone: R¹=Me, R²=H, R³=Me;
Gamma tocopherol hydroquinone: R¹=H, R²=Me, R³=Me;
Delta tocopherol hydroquinone: R¹=H, R²=H, R³=Me;

The following structure shows the structure of alpha-tocopherol, wherein an * symbol represents a chiral center:

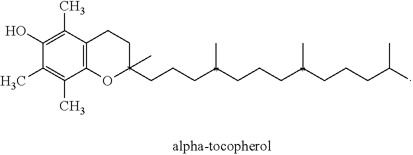

All eight stereoisomers of alpha-tocopherol are as follows:

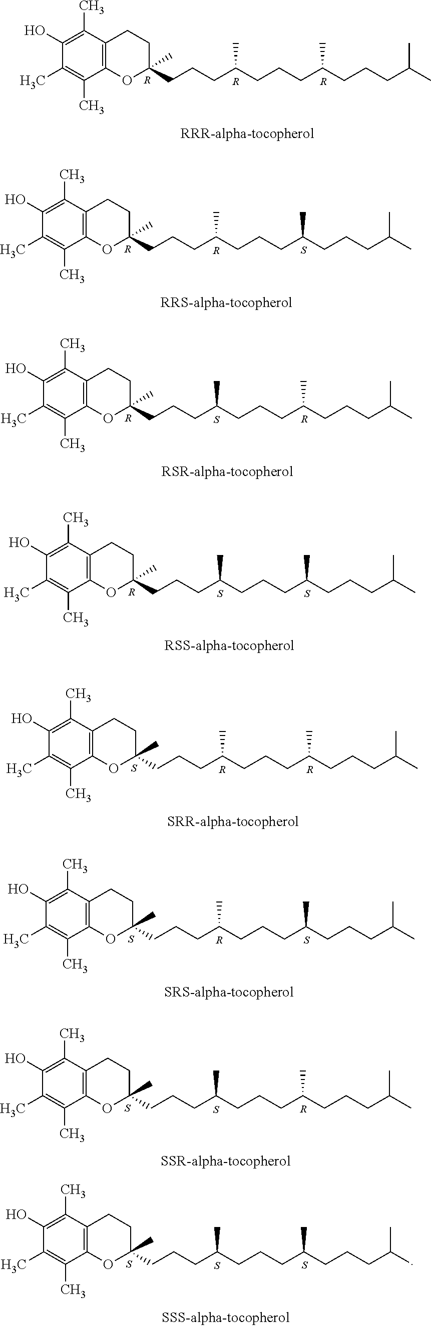

All eight stereoisomers of alpha-tocopherol quinone are as follows:

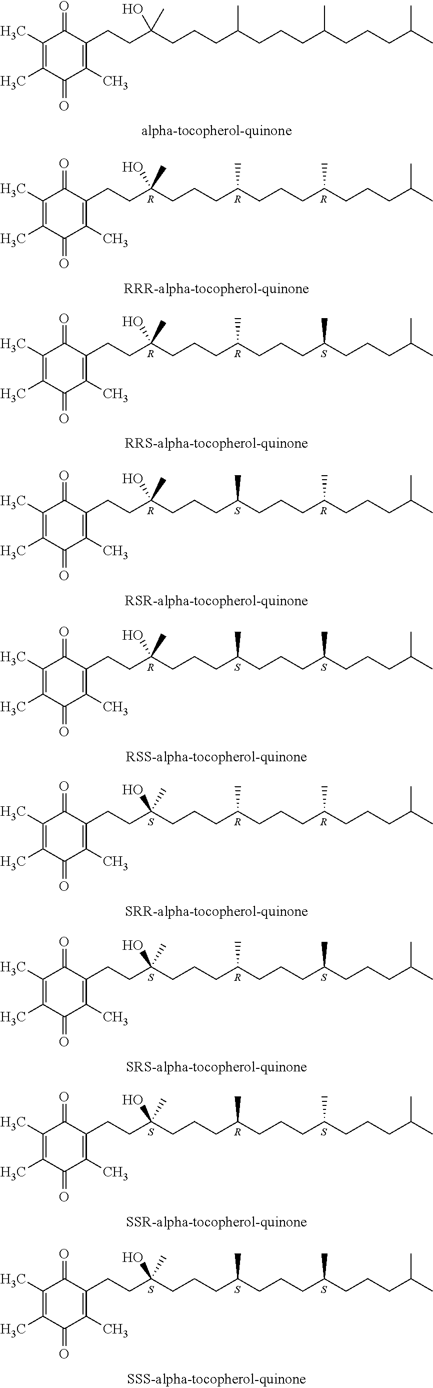

The eight stereoisomers of alpha-tocotrienol are as follows:

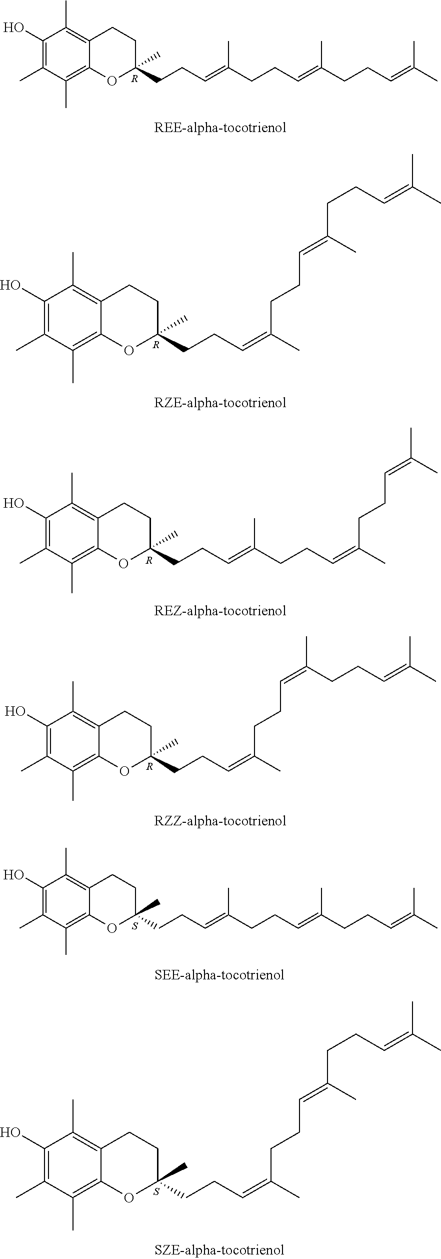

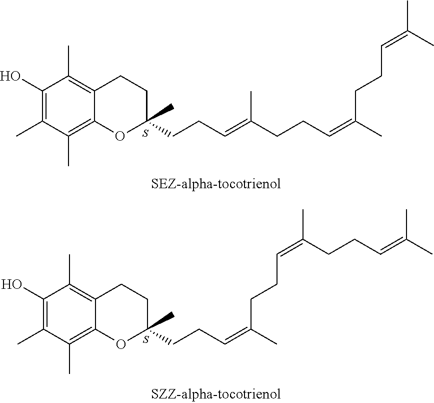

Compositions enriched in metabolites of the stereoisomers of the tocopherol and tocotrienol compounds described herein are also contemplated. Tocopherol quinones are metabolites of tocopherol compounds. Tocotrienol quinones are metabolites of tocotrienol compounds. In some embodiments, these quinones (or their hydroquinone forms thereof) have improved biological activity over their respective chroman compounds. Thus, chroman stereoisomers that have more rapid and/or more complete conversion to their respective quinone or hydroquinone may be preferred in some embodiments over other chroman stereoisomers.

### Non-Limiting Examples of Additional Metabolites

Non-limiting examples of metabolites of (R,E,E)-alpha-tocotrienol include the following:

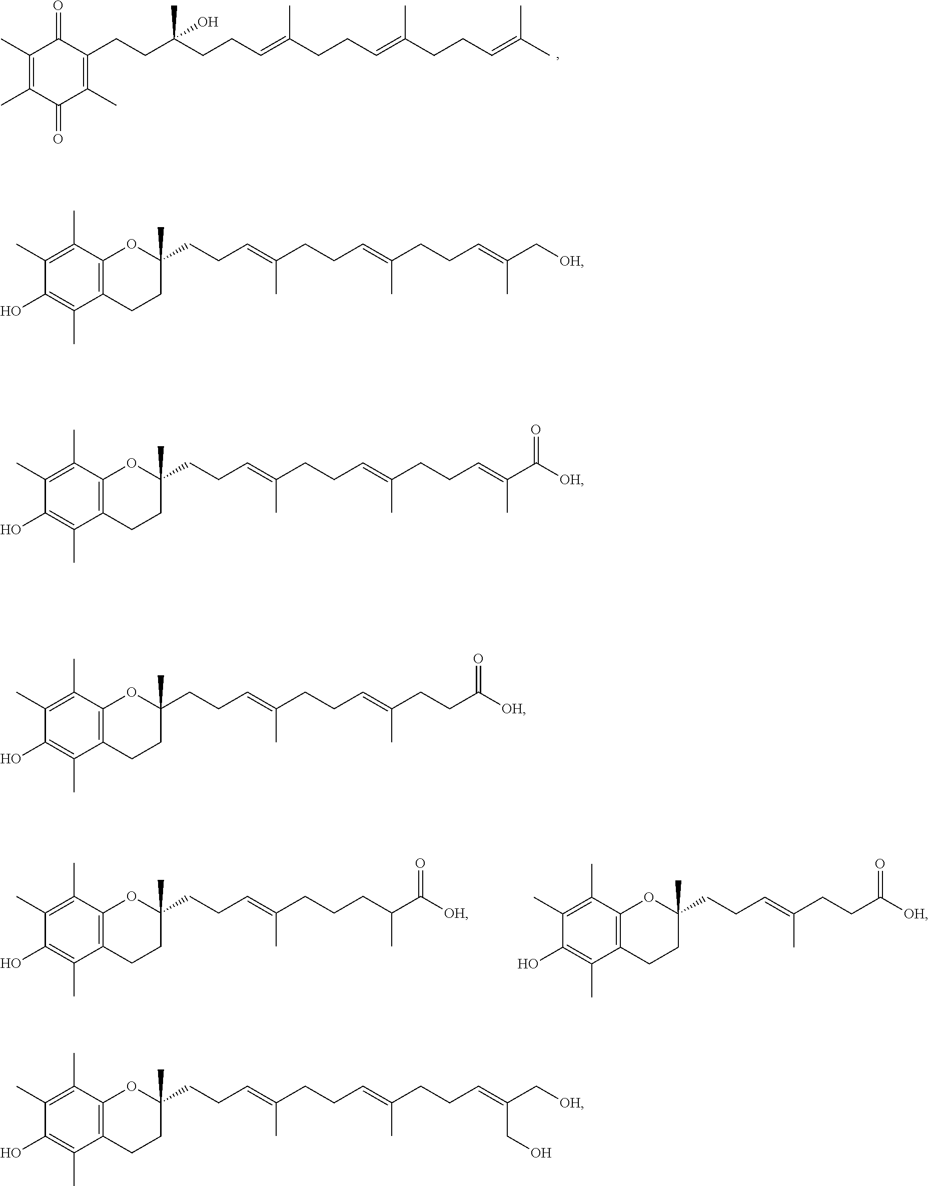

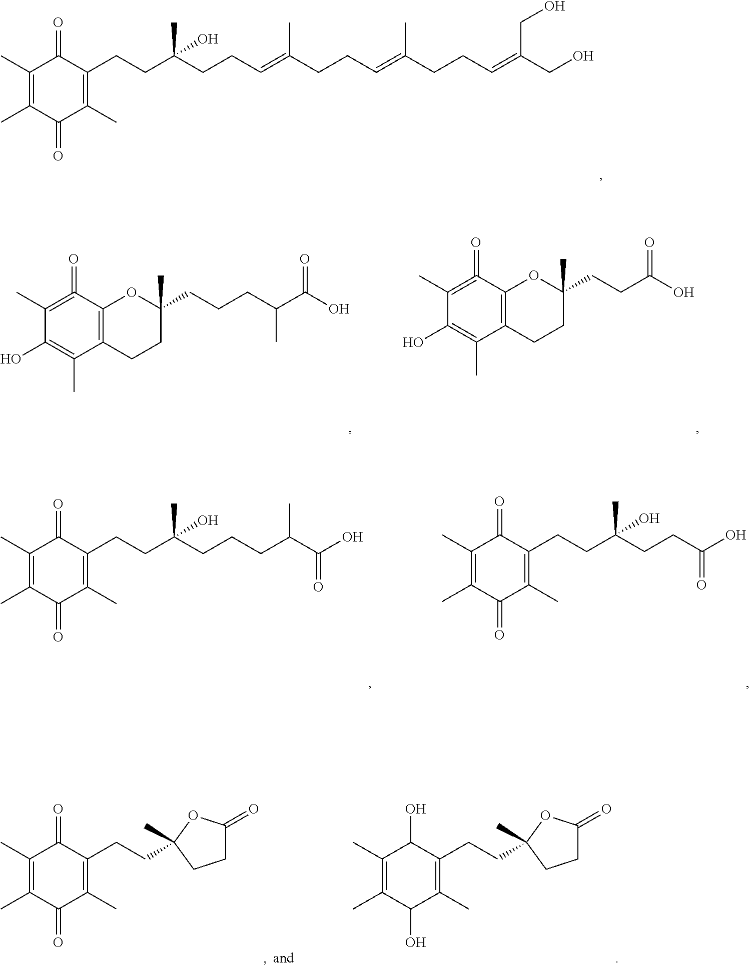

Metabolites of (R,E,E)-alpha tocotrienol also include metabolites of (R,E,E)-alpha tocotrienol quinone.

Non-limiting examples of metabolites of (R,E,E)-alpha-tocotrienol quinone include the following:

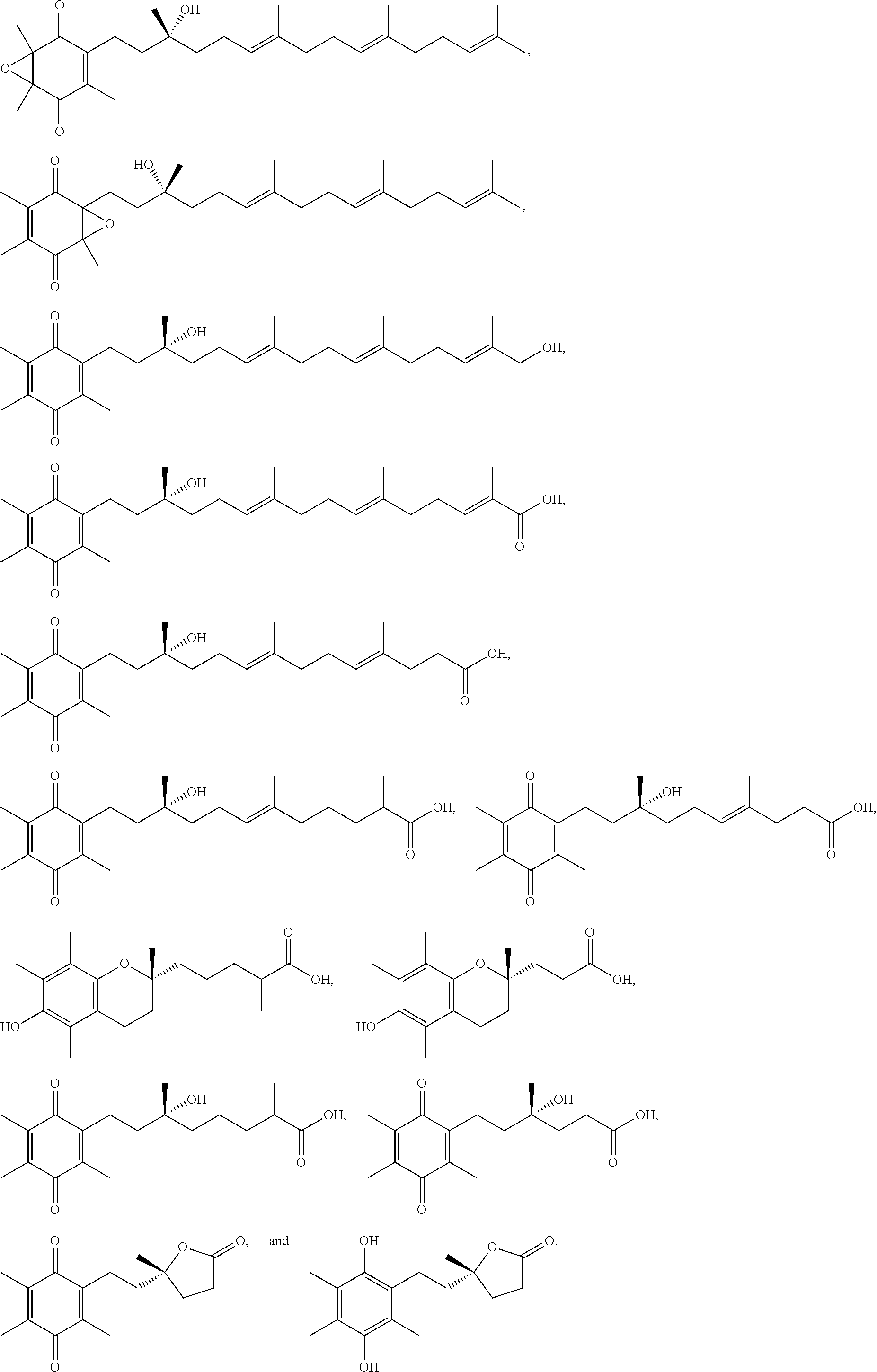

Non-limiting examples of metabolites of (R,R,R)-alpha-tocopherol include the following:

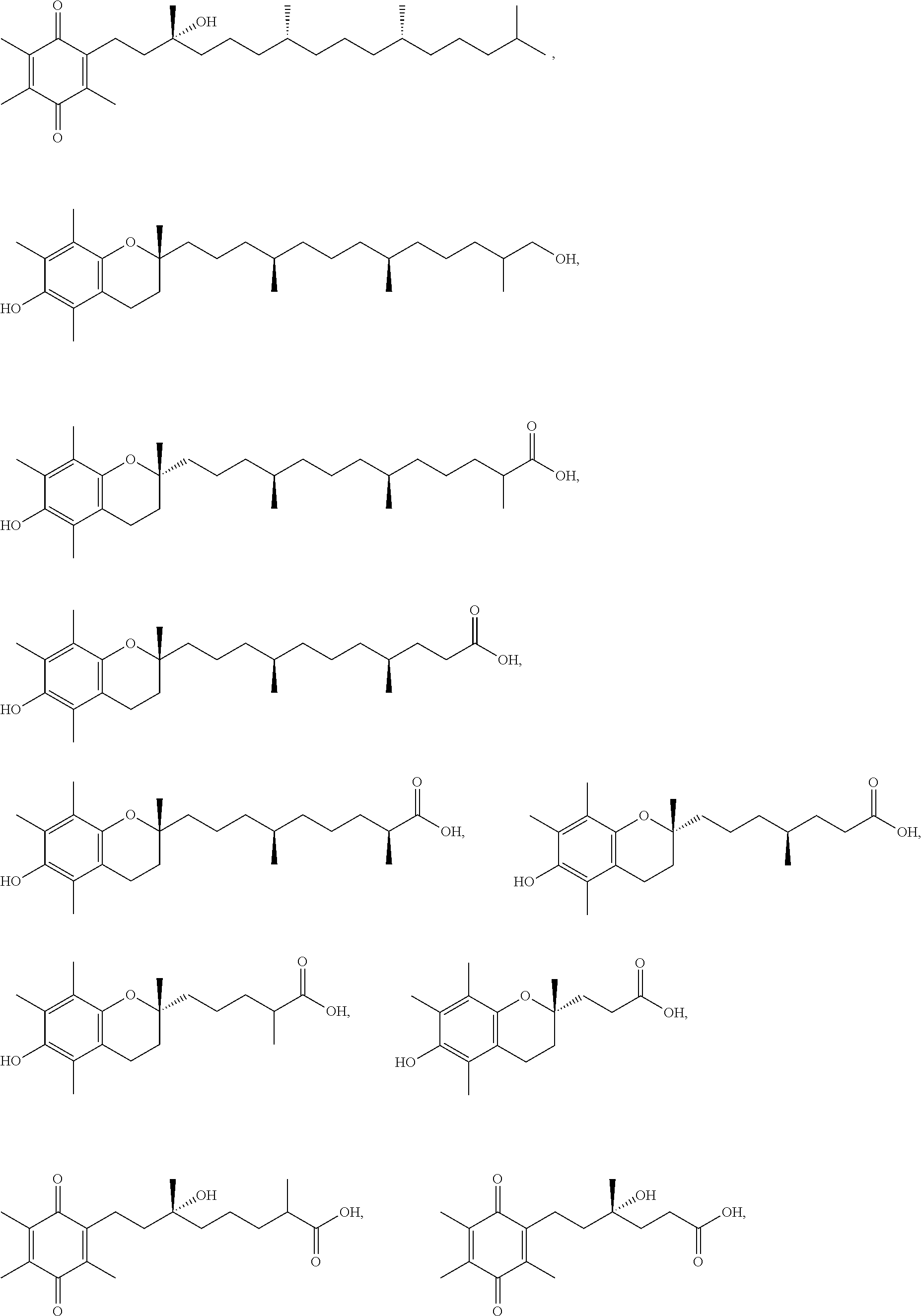

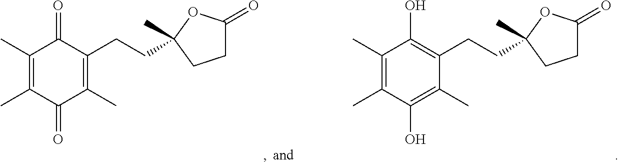 [SMILES: CC1=C(C)C(=O)C(CC[C@@]2(C)CCC(=O)O2)=C(C)C1=O ; Cc1c(C)c(O)c(CC[C@@]2(C)CCC(=O)O2)c(C)c1O]

Metabolites of (R,R,R)-alpha tocopherol also include metabolites of (R,R,R)-alpha tocopherol quinone.

Non-limiting examples of metabolites of (R,R,R)-alpha tocopherol quinone include the following:

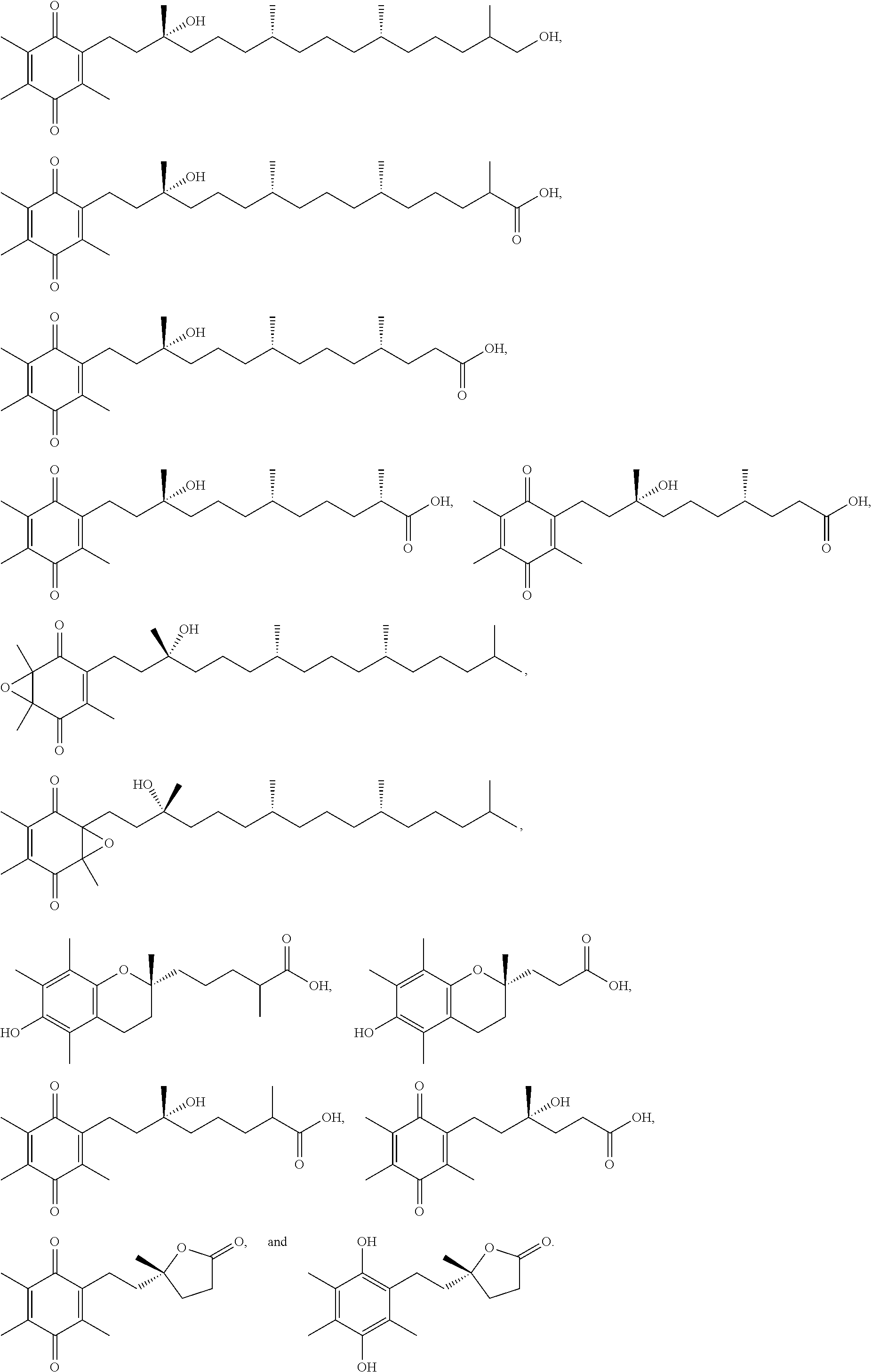

Although the compositions are described as being enriched in or comprising substantially pure synthetic, non-plant based stereoisomer(s) of the above-described compounds, in the case of certain compounds for use in certain applications or in formulations or co-administrations, the composition may further comprise one or more of the above described compounds which may be either synthetic or non-synthetic, plant-based or non-plant based, and may or may not be substantially pure isomers. For example, when the composition is a food to which substantially pure synthetic, non-plant based RRR-alpha tocopherol has been added, there may additionally be naturally occurring plant-based RRR-alpha tocopherol present, for example, in a food containing palm oil. As an additional example, a formulation may comprise substantially pure synthetic, non-plant based RRR-alpha tocopherol, and may additionally comprise beta tocopherol that is plant based, naturally occurring, and/or all-racemic.

### Formulations

The compound compositions described above may, in some embodiments, be incorporated in a broader formulation or composition, e.g., formulated with other components, to provide for different applications and to serve different purposes, such as to improve bioavailability, improve storage, controlled release, solubility, mode of administration, and the like. Non-limiting examples of such compositions include food products, medical foods, functional foods, nutritional supplements, vitamin supplements, pharmaceutical formulations, cosmetic formulations, etc. The compositions may be formulated for enteral, oral, parenteral, sublingual, inhalation (e.g. as mists or sprays), rectal, or topical use.

In some embodiments, the compositions described herein may be formulated as an admixture with edible oils (such as corn oil, cottonseed oil, sesame oil, coconut oil) or propylene glycol, e.g. for oral administration (e.g. in gelatin capsules), intraperitoneal administration, or subcutaneous administration.

In some embodiments, the compositions may be adsorbed by admixture to a solid matrix, such as calcium phosphate, calcium sulfate, starches, modified starches, microcrystalline cellulose, micro cellulose, or talcum. In some embodiments, the adsorbed solid may be milled into a free flowing powder and filled into hard gelatin capsules, e.g., for oral administration.

In still other embodiments, the composition may be admixed with petroleum jelly, dissolved in dimethylsulfoxide (DMSO), or it may be combined with an emulsifier to provide an oil in water emulsion, e.g. for use in topical or oral administration. Non-limiting examples of suitable emulsifiers include sodium lauryl sulfate, sorbitan monooleate, polyoxyethylene sorbitan monooleate, polysorbates, polaxomers, bile salts, glyceryl monostearate, copolymers of ethylene oxide and propylene oxide.

In certain embodiments, e.g., for medical food or clinical food applications, the compositions described herein may be formulated with one or more of dietary carbohydrates, e.g., sugars and starches, dietary fats and dietary proteins, e.g., whey proteins, soy proteins and the like. In some embodiments, these formulations may be in conjunction with one or more of the formulations described above.

In some embodiments, a composition may comprise one or more synthetic, non-plant based stereoisomers of alpha-, beta-, gamma-, or delta- tocopherol, tocotrienol, tocopherol quinone, tocotrienol quinone, tocopherol hydroquinone, tocotrienol hydroquinone, or a metabolite thereof. In some embodiments, the tocopherol, tocopherol quinone, or tocopherol hydroquinone isomer is RRR. In some embodiments, the tocopherol, tocopherol quinone, or tocopherol hydroquinone isomer(s) is R, S/R, S/R, wherein S/R indicates either or both configurations at that position. In some embodiments, the tocotrienol, tocotrienol quinone, or tocotrienol hydroquinone isomer is REE. In some embodiments, a specific stereoisomer(s) of a compound can be present in a composition in an amount of at least about 0.0001 weight %, 0.001 weight %, 0.01 weight %, 0.1 weight %, 0.2 weight %, 0.3 weight %, 0.4 weight %, 0.5 weight %, 0.6 weight %, 0.7 weight %, 0.8 weight %, 0.9 weight %, 1 weight %, 2 weight %, 3 weight %, 4 weight %, 5 weight %, 6 weight %, 7 weight %, 8 weight %, 9 weight %, 10 weight %, 11 weight %, 12 weight %, 13 weight %, 14 weight %, 15 weight %, 16 weight %, 17 weight %, 18 weight %, 19 weight %, 20 weight %, 21 weight %, 22 weight %, 23 weight %, 24 weight %, 25 weight %, 26 weight %, 27 weight %, 28 weight %, 29 weight %, 30 weight %, 35 weight %, 40 weight %, 50 weight %, 60 weight %, 70 weight %, 80 weight %, 90 weight %, 95 weight %, 96 weight %, 97 weight %, 98 weight %, 99 weight %, 99.5 weight percent, or 99.9 weight %, wherein the weight % of the stereoisomer(s) is based on a total weight % of the composition. In some embodiments, a specific stereoisomer(s) of a compound can be present in a composition in an amount of at most about 0.0001 weight %, 0.001 weight %, 0.01 weight %, 0.1 weight %, 0.2 weight %, 0.3 weight %, 0.4 weight %, 0.5 weight %, 0.6 weight %, 0.7 weight %, 0.8 weight %, 0.9 weight %, 1 weight %, 2 weight %, 3 weight %, 4 weight %, 5 weight %, 6 weight %, 7 weight %, 8 weight %, 9 weight %, 10 weight %, 11 weight %, 12 weight %, 13 weight %, 14 weight %, 15 weight %, 16 weight %, 17 weight %, 18 weight %, 19 weight %, 20 weight %, 21 weight %, 22 weight %, 23 weight %, 24 weight %, 25 weight %, 26 weight %, 27 weight %, 28 weight %, 29 weight %, 30 weight %, 35 weight %, 40 weight %, 50 weight %, 60 weight %, 70 weight %, 80 weight %, 90 weight %, 95 weight %, 96 weight %, 97 weight %, 98 weight %, 99 weight %, 99.5 weight percent, or 99.9 weight %, wherein the weight % of the stereoisomer(s) is based on a total weight % of the composition. In some embodiments, the specific stereoisomer(s) of a compound is present in a composition at about 55 weight %-99.9 weight %, about 80 weight %-99 weight %, or about 90 weight %-95 weight %, wherein the weight % of the isomer is based on a total weight % of all stereoisomers of the compound combined. In some or any embodiments in this paragraph, “weight %” is replaced by “mol %.”

In some embodiments, RRR-alpha tocopherol is present in a composition in an amount of at least about 0.0001 weight %, 0.001 weight %, 0.01 weight %, 0.1 weight %, 0.2 weight %, 0.3 weight %, 0.4 weight %, 0.5 weight %, 0.6 weight %, 0.7 weight %, 0.8 weight %, 0.9 weight %, 1 weight %, 2 weight %, 3 weight %, 4 weight %, 5 weight %, 6 weight %, 7 weight %, 8 weight %, 9 weight %, 10 weight %, 11 weight %, 12 weight %, 13 weight %, 14 weight %, 15 weight %, 16 weight %, 17 weight %, 18 weight %, 19 weight %, 20 weight %, 21 weight %, 22 weight %, 23 weight %, 24 weight %, 25 weight %, 26 weight %, 27 weight %, 28 weight %, 29 weight %, 30 weight %, 35 weight %, 40 weight %, 50 weight %, 60 weight %, 70 weight %, 80 weight %, 90 weight %, 95 weight %, 96 weight %, 97 weight %, 98 weight %, 99 weight %, 99.5 weight percent, or 99.9 weight %, wherein the weight % of the stereoisomer(s) is based on a total weight % of the composition. In some embodiments, RRR-alpha tocopherol is present in a composition in an amount of at most about 0.0001 weight %, 0.001 weight %, 0.01 weight %, 0.1 weight %, 0.2 weight %, 0.3 weight %, 0.4 weight %, 0.5 weight %, 0.6 weight %, 0.7 weight %, 0.8 weight %, 0.9 weight %, 1 weight %, 2 weight %, 3 weight %, 4 weight %, 5 weight %, 6 weight %, 7 weight %, 8 weight %, 9 weight %, 10 weight %, 11 weight %, 12 weight %, 13 weight %, 14 weight %, 15 weight %, 16 weight %, 17 weight %, 18 weight %, 19 weight %, 20 weight %, 21 weight %, 22 weight %, 23 weight %, 24 weight %, 25 weight %, 26 weight %, 27 weight %, 28 weight %, 29 weight %, 30 weight %, 35 weight %, 40 weight %, 50 weight %, 60 weight %, 70 weight %, 80 weight %, 90 weight %, 95 weight %, 96 weight %, 97 weight %, 98 weight %, 99 weight %, 99.5 weight percent, or 99.9 weight %, wherein the weight % of the stereoisomer(s) is based on a total weight % of the composition. In some embodiments, the RRR-isomer of alpha-tocopherol is present in a composition at about 55 weight %-99.9 weight %, about 80 weight %-99 weight %, or about 90 weight %-95 weight %, wherein the weight % of the isomer is based on a total weight % of all stereoisomers of alpha-tocopherol combined. In some or any embodiments in this paragraph, “weight %” is replaced by “mol %.”

In some embodiments, RRR-alpha tocopherol quinone is present in a composition in an amount of at least about 0.0001 weight %, 0.001 weight %, 0.01 weight %, 0.1 weight %, 0.2 weight %, 0.3 weight %, 0.4 weight %, 0.5 weight %, 0.6 weight %, 0.7 weight %, 0.8 weight %, 0.9 weight %, 1 weight %, 2 weight %, 3 weight %, 4 weight %, 5 weight %, 6 weight %, 7 weight %, 8 weight %, 9 weight %, 10 weight %, 11 weight %, 12 weight %, 13 weight %, 14 weight %, 15 weight %, 16 weight %, 17 weight %, 18 weight %, 19 weight %, 20 weight %, 21 weight %, 22 weight %, 23 weight %, 24 weight %, 25 weight %, 26 weight %, 27 weight %, 28 weight %, 29 weight %, 30 weight %, 35 weight %, 40 weight %, 50 weight %, 60 weight %, 70 weight %, 80 weight %, 90 weight %, 95 weight %, 96 weight %, 97 weight %, 98 weight %, 99 weight %, 99.5 weight percent, or 99.9 weight %, wherein the weight % of the stereoisomer(s) is based on a total weight % of the composition. In some embodiments, RRR-alpha tocopherol quinone is present in a composition in an amount of at most about 0.0001 weight %, 0.001 weight %, 0.01 weight %, 0.1 weight %, 0.2 weight %, 0.3 weight %, 0.4 weight %, 0.5 weight %, 0.6 weight %, 0.7 weight %, 0.8 weight %, 0.9 weight %, 1 weight %, 2 weight %, 3 weight %, 4 weight %, 5 weight %, 6 weight %, 7 weight %, 8 weight %, 9 weight %, 10 weight %, 11 weight %, 12 weight %, 13 weight %, 14 weight %, 15 weight %, 16 weight %, 17 weight %, 18 weight %, 19 weight %, 20 weight %, 21 weight %, 22 weight %, 23 weight %, 24 weight %, 25 weight %, 26 weight %, 27 weight %, 28 weight %, 29 weight %, 30 weight %, 35 weight %, 40 weight %, 50 weight %, 60 weight %, 70 weight %, 80 weight %, 90 weight %, 95 weight %, 96 weight %, 97 weight %, 98 weight %, 99 weight %, 99.5 weight percent, or 99.9 weight %, wherein the weight % of the stereoisomer(s) is based on a total weight % of the composition. In some embodiments, the RRR-isomer of alpha-tocopherol quinone is present in a composition at about 55 weight %-99.9 weight %, about 80 weight %-99 weight %, or about 90 weight %-95 weight %, wherein the weight % of the isomer is based on a total weight % of all stereoisomers of alpha-tocopherol quinone combined. In some or any embodiments in this paragraph, “weight %” is replaced by “mol %.”

In some embodiments, RRR-alpha tocopherol hydroquinone is present in a composition in an amount of at least about 0.0001 weight %, 0.001 weight %, 0.01 weight %, 0.1 weight %, 0.2 weight %, 0.3 weight %, 0.4 weight %, 0.5 weight %, 0.6 weight %, 0.7 weight %, 0.8 weight %, 0.9 weight %, 1 weight %, 2 weight %, 3 weight %, 4 weight %, 5 weight %, 6 weight %, 7 weight %, 8 weight %, 9 weight %, 10 weight %, 11 weight %, 12 weight %, 13 weight %, 14 weight %, 15 weight %, 16 weight %, 17 weight %, 18 weight %, 19 weight %, 20 weight %, 21 weight %, 22 weight %, 23 weight %, 24 weight %, 25 weight %, 26 weight %, 27 weight %, 28 weight %, 29 weight %, 30 weight %, 35 weight %, 40 weight %, 50 weight %, 60 weight %, 70 weight %, 80 weight %, 90 weight %, 95 weight %, 96 weight %, 97 weight %, 98 weight %, 99 weight %, 99.5 weight percent, or 99.9 weight %, wherein the weight % of the stereoisomer(s) is based on a total weight % of the composition. In some embodiments, RRR-alpha tocopherol hydroquinone is present in a composition in an amount of at most about 0.0001 weight %, 0.001 weight %, 0.01 weight %, 0.1 weight %, 0.2 weight %, 0.3 weight %, 0.4 weight %, 0.5 weight %, 0.6 weight %, 0.7 weight %, 0.8 weight %, 0.9 weight %, 1 weight %, 2 weight %, 3 weight %, 4 weight %, 5 weight %, 6 weight %, 7 weight %, 8 weight %, 9 weight %, 10 weight %, 11 weight %, 12 weight %, 13 weight %, 14 weight %, 15 weight %, 16 weight %, 17 weight %, 18 weight %, 19 weight %, 20 weight %, 21 weight %, 22 weight %, 23 weight %, 24 weight %, 25 weight %, 26 weight %, 27 weight %, 28 weight %, 29 weight %, 30 weight %, 35 weight %, 40 weight %, 50 weight %, 60 weight %, 70 weight %, 80 weight %, 90 weight %, 95 weight %, 96 weight %, 97 weight %, 98 weight %, 99 weight %, 99.5 weight percent, or 99.9 weight %, wherein the weight % of the stereoisomer(s) is based on a total weight % of the composition. In some embodiments, the RRR- isomer of alpha-tocopherol hydroquinone is present in a composition at about 55 weight %-99.9 weight %, about 80 weight %-99 weight %, or about 90 weight %-95 weight %, wherein the weight % of the isomer is based on a total weight % of all stereoisomers of alpha-tocopherol hydroquinone combined. In some or any embodiments in this paragraph, “weight %” is replaced by “mol %.”

In some embodiments, REE-alpha tocotrienol is present in a composition in an amount of at least about 0.0001 weight %, 0.001 weight %, 0.01 weight %, 0.1 weight %, 0.2 weight %, 0.3 weight %, 0.4 weight %, 0.5 weight %, 0.6 weight %, 0.7 weight %, 0.8 weight %, 0.9 weight %, 1 weight %, 2 weight %, 3 weight %, 4 weight %, 5 weight %, 6 weight %, 7 weight %, 8 weight %, 9 weight %, 10 weight %, 11 weight %, 12 weight %, 13 weight %, 14 weight %, 15 weight %, 16 weight %, 17 weight %, 18 weight %, 19 weight %, 20 weight %, 21 weight %, 22 weight %, 23 weight %, 24 weight %, 25 weight %, 26 weight %, 27 weight %, 28 weight %, 29 weight %, 30 weight %, 35 weight %, 40 weight %, 50 weight %, 60 weight %, 70 weight %, 80 weight %, 90 weight %, 95 weight %, 96 weight %, 97 weight %, 98 weight %, 99 weight %, 99.5 weight percent, or 99.9 weight %, wherein the weight % of the stereoisomer(s) is based on a total weight % of the composition. In some embodiments, REE-alpha tocotrienol is present in a composition in an amount of at most about 0.0001 weight %, 0.001 weight %, 0.01 weight %, 0.1 weight %, 0.2 weight %, 0.3 weight %, 0.4 weight %, 0.5 weight %, 0.6 weight %, 0.7 weight %, 0.8 weight %, 0.9 weight %, 1 weight %, 2 weight %, 3 weight %, 4 weight %, 5 weight %, 6 weight %, 7 weight %, 8 weight %, 9 weight %, 10 weight %, 11 weight %, 12 weight %, 13 weight %, 14 weight %, 15 weight %, 16 weight %, 17 weight %, 18 weight %, 19 weight %, 20 weight %, 21 weight %, 22 weight %, 23 weight %, 24 weight %, 25 weight %, 26 weight %, 27 weight %, 28 weight %, 29 weight %, 30 weight %, 35 weight %, 40 weight %, 50 weight %, 60 weight %, 70 weight %, 80 weight %, 90 weight %, 95 weight %, 96 weight %, 97 weight %, 98 weight %, 99 weight %, 99.5 weight percent, or 99.9 weight %, wherein the weight % of the stereoisomer(s) is based on a total weight % of the composition. In some embodiments, the REE-isomer of alpha-tocotrienol is present in a composition at about 55 weight %-99.9 weight %, about 80 weight %-99 weight %, or about 90 weight %-95 weight %, wherein the weight % of the isomer is based on a total weight % of all stereoisomers of alpha-tocotrienol combined. In some or any embodiments in this paragraph, “weight %” is replaced by “mol %.”

In some embodiments, REE-alpha tocotrienol quinone is present in a composition in an amount of at least about 0.0001 weight %, 0.001 weight %, 0.01 weight %, 0.1 weight %, 0.2 weight %, 0.3 weight %, 0.4 weight %, 0.5 weight %, 0.6 weight %, 0.7 weight %, 0.8 weight %, 0.9 weight %, 1 weight %, 2 weight %, 3 weight %, 4 weight %, 5 weight %, 6 weight %, 7 weight %, 8 weight %, 9 weight %, 10 weight %, 11 weight %, 12 weight %, 13 weight %, 14 weight %, 15 weight %, 16 weight %, 17 weight %, 18 weight %, 19 weight %, 20 weight %, 21 weight %, 22 weight %, 23 weight %, 24 weight %, 25 weight %, 26 weight %, 27 weight %, 28 weight %, 29 weight %, 30 weight %, 35 weight %, 40 weight %, 50 weight %, 60 weight %, 70 weight %, 80 weight %, 90 weight %, 95 weight %, 96 weight %, 97 weight %, 98 weight %, 99 weight %, 99.5 weight percent, or 99.9 weight %, wherein the weight % of the stereoisomer(s) is based on a total weight % of the composition. In some embodiments, REE-alpha tocotrienol quinone is present in a composition in an amount of at most about 0.0001 weight %, 0.001 weight %, 0.01 weight %, 0.1 weight %, 0.2 weight %, 0.3 weight %, 0.4 weight %, 0.5 weight %, 0.6 weight %, 0.7 weight %, 0.8 weight %, 0.9 weight %, 1 weight %, 2 weight %, 3 weight %, 4 weight %, 5 weight %, 6 weight %, 7 weight %, 8 weight %, 9 weight %, 10 weight %, 11 weight %, 12 weight %, 13 weight %, 14 weight %, 15 weight %, 16 weight %, 17 weight %, 18 weight %, 19 weight %, 20 weight %, 21 weight %, 22 weight %, 23 weight %, 24 weight %, 25 weight %, 26 weight %, 27 weight %, 28 weight %, 29 weight %, 30 weight %, 35 weight %, 40 weight %, 50 weight %, 60 weight %, 70 weight %, 80 weight %, 90 weight %, 95 weight %, 96 weight %, 97 weight %, 98 weight %, 99 weight %, 99.5 weight percent, or 99.9 weight %, wherein the weight % of the stereoisomer(s) is based on a total weight % of the composition. In some embodiments, the REE-isomer of alpha-tocotrienol quinone is present in a composition at about 55 weight %-99.9 weight %, about 80 weight %-99 weight %, or about 90 weight %-95 weight %, wherein the weight % of the isomer is based on a total weight % of all stereoisomers of alpha-tocotrienol quinone combined. In some or any embodiments in this paragraph, “weight %” is replaced by “mol %.”

In some embodiments, REE-alpha tocotrienol hydroquinone is present in a composition in an amount of at least about 0.0001 weight %, 0.001 weight %, 0.01 weight %, 0.1 weight %, 0.2 weight %, 0.3 weight %, 0.4 weight %, 0.5 weight %, 0.6 weight %, 0.7 weight %, 0.8 weight %, 0.9 weight %, 1 weight %, 2 weight %, 3 weight %, 4 weight %, 5 weight %, 6 weight %, 7 weight %, 8 weight %, 9 weight %, 10 weight %, 11 weight %, 12 weight %, 13 weight %, 14 weight %, 15 weight %, 16 weight %, 17 weight %, 18 weight %, 19 weight %, 20 weight %, 21 weight %, 22 weight %, 23 weight %, 24 weight %, 25 weight %, 26 weight %, 27 weight %, 28 weight %, 29 weight %, 30 weight %, 35 weight %, 40 weight %, 50 weight %, 60 weight %, 70 weight %, 80 weight %, 90 weight %, 95 weight %, 96 weight %, 97 weight %, 98 weight %, 99 weight %, 99.5 weight percent, or 99.9 weight %, wherein the weight % of the stereoisomer(s) is based on a total weight % of the composition. In some embodiments, REE-alpha tocotrienol hydroquinone is present in a composition in an amount of at most about 0.0001 weight %, 0.001 weight %, 0.01 weight %, 0.1 weight %, 0.2 weight %, 0.3 weight %, 0.4 weight %, 0.5 weight %, 0.6 weight %, 0.7 weight %, 0.8 weight %, 0.9 weight %, 1 weight %, 2 weight %, 3 weight %, 4 weight %, 5 weight %, 6 weight %, 7 weight %, 8 weight %, 9 weight %, 10 weight %, 11 weight %, 12 weight %, 13 weight %, 14 weight %, 15 weight %, 16 weight %, 17 weight %, 18 weight %, 19 weight %, 20 weight %, 21 weight %, 22 weight %, 23 weight %, 24 weight %, 25 weight %, 26 weight %, 27 weight %, 28 weight %, 29 weight %, 30 weight %, 35 weight %, 40 weight %, 50 weight %, 60 weight %, 70 weight %, 80 weight %, 90 weight %, 95 weight %, 96 weight %, 97 weight %, 98 weight %, 99 weight %, 99.5 weight percent, or 99.9 weight %, wherein the weight % of the stereoisomer(s) is based on a total weight % of the composition. In some embodiments, the REE-isomer of alpha-tocotrienol hydroquinone is present in a composition at about 55 weight %-99.9 weight %, about 80 weight %-99 weight %, or about 90 weight %-95 weight %, wherein the weight % of the isomer is based on a total weight % of all stereoisomers of alpha-tocotrienol hydroquinone combined. In some or any embodiments in this paragraph, “weight %” is replaced by “mol %.”

In some embodiments, a specific stereoisomer(s) of a compound can be present in a composition in a positive amount and less than about 0.01 weight %, 0.1 weight %, 0.2 weight %, 0.3 weight %, 0.4 weight %, 0.5 weight %, 0.6 weight %, 0.7 weight %, 0.8 weight %, 0.9 weight %, 1 weight %, 2 weight %, 3 weight %, 4 weight %, 5 weight %, 6 weight %, 7 weight %, 8 weight %, 9 weight %, 10 weight %, 11 weight %, 12 weight %, 13 weight %, 14 weight %, 15 weight %, 16 weight %, 17 weight %, 18 weight %, 19 weight %, 20 weight %, 21 weight %, 22 weight %, 23 weight %, 24 weight %, 25 weight %, 26 weight %, 27 weight %, 28 weight %, 29 weight %, 30 weight %, 35 weight %, 40 weight %, 50 weight %, 60 weight %, 70 weight %, 80 weight %, 90 weight %, or 95 weight %, wherein the weight % of the stereoisomer(s) is based on a total weight % of the composition. In some or any embodiments in this paragraph, “weight %” is replaced by “mol %.”

In some embodiments, a composition may comprise a substantially pure single stereoisomer and comprise less than about 10%, 9%, 8%, 7%, 6%, 5%, 4%, 3%, 2%, 1%, or 0.5% of all other stereoisomers of that compound combined, wherein % is weight %. In some embodiments, a composition comprises a substantially pure single stereoisomer of alpha tocopherol, and comprises less than about 10%, 9%, 8%, 7%, 6%, 5%, 4%, 3%, 2%, 1%, or 0.5% of all other stereoisomers of alpha tocopherol combined. In some embodiments, a composition comprises a substantially pure single stereoisomer of alpha tocopherol quinone, and comprises less than about 10%, 9%, 8%, 7%, 6%, 5%, 4%, 3%, 2%, 1%, or 0.5% of all other stereoisomers of alpha tocopherol quinone combined. In some embodiments, a composition comprises a substantially pure single stereoisomer of alpha tocopherol hydroquinone, and comprises less than about 10%, 9%, 8%, 7%, 6%, 5%, 4%, 3%, 2%, 1%, or 0.5% of all other stereoisomers of alpha tocopherol hydroquinone combined. In some embodiments, a composition comprises a substantially pure single stereoisomer of alpha tocotrienol, and comprises less than about 10%, 9%, 8%, 7%, 6%, 5%, 4%, 3%, 2%, 1%, or 0.5% of all other stereoisomers of alpha tocotrienol combined. In some embodiments, a composition comprises a substantially pure single stereoisomer of alpha tocotrienol quinone, and comprises less than about 10%, 9%, 8%, 7%, 6%, 5%, 4%, 3%, 2%, 1%, or 0.5% of all other stereoisomers of alpha tocotrienol quinone combined. In some embodiments, a composition comprises a substantially pure single stereoisomer of alpha tocotrienol hydroquinone, and comprises less than about 10%, 9%, 8%, 7%, 6%, 5%, 4%, 3%, 2%, 1%, or 0.5% of all other stereoisomers of alpha tocotrienol hydroquinone combined.

In some embodiments, a composition may be substantially free of one or more stereoisomer(s), comprising less than about 10%, 9%, 8%, 7%, 6%, 5%, 4%, 3%, 2%, 1%, or 0.5% of those stereoisomer(s), when compared as a weight % relative to all stereoisomers of that compound combined. In some embodiments, a composition is substantially free of S, S/R, S/R isomers of a tocopherol or tocopherol quinone or hydroquinone (wherein S/R indicates either or both configurations at that position), comprising less than about 10%, 9%, 8%, 7%, 6%, 5%, 4%, 3%, 2%, 1%, or 0.5% of those stereoisomer(s), when compared as a weight % relative to all stereoisomers of the tocopherol or tocopherol quinone or hydroquinone combined. In some embodiments, a composition is substantially free of S, Z/E, Z/E isomers of a tocotrienol or tocotrienol quinone or hydroquinone (wherein Z/E indicates either or both configurations at that position), comprising less than about 10%, 9%, 8%, 7%, 6%, 5%, 4%, 3%, 2%, 1%, or 0.5% of those stereoisomer(s), when compared as a weight % relative to all stereoisomers of the tocotrienol or tocotrienol quinone or hydroquinone combined. In some embodiments, a composition is substantially free of S, S/R, S/R isomers of alpha tocopherol (wherein S/R indicates either or both configurations at that position), comprising less than about 10%, 9%, 8%, 7%, 6%, 5%, 4%, 3%, 2%, 1%, or 0.5% of those stereoisomer(s), when compared as a weight % relative to all stereoisomers of alpha tocopherol combined. In some embodiments, a composition is substantially free of S, S/R, S/R isomers of alpha tocopherol quinone (wherein S/R indicates either or both configurations at that position), comprising less than about 10%, 9%, 8%, 7%, 6%, 5%, 4%, 3%, 2%, 1%, or 0.5% of those stereoisomer(s), when compared as a weight % relative to all stereoisomers of alpha tocopherol quinone combined. In some embodiments, a composition is substantially free of S, S/R, S/R isomers of alpha tocopherol hydroquinone (wherein S/R indicates either or both configurations at that position), comprising less than about 10%, 9%, 8%, 7%, 6%, 5%, 4%, 3%, 2%, 1%, or 0.5% of those stereoisomer(s), when compared as a weight % relative to all stereoisomers of alpha tocopherol hydroquinone combined. In some embodiments, a composition is substantially free of S, Z/E, Z/E isomers of alpha tocotrienol (wherein Z/E indicates either or both configurations at that position), comprising less than about 10%, 9%, 8%, 7%, 6%, 5%, 4%, 3%, 2%, 1%, or 0.5% of those stereoisomer(s), when compared as a weight % relative to all stereoisomers of alpha tocotrienol combined. In some embodiments, a composition is substantially free of S, Z/E, Z/E of alpha tocotrienol quinone (wherein Z/E indicates either or both configurations at that position), comprising less than about 10%, 9%, 8%, 7%, 6%, 5%, 4%, 3%, 2%, 1%, or 0.5% of those stereoisomer(s), when compared as a weight % relative to all stereoisomers of alpha tocotrienol quinone combined. In some embodiments, a composition is substantially free of S, Z/E, Z/E of alpha tocotrienol hydroquinone (wherein Z/E indicates either or both configurations at that position), comprising less than about 10%, 9%, 8%, 7%, 6%, 5%, 4%, 3%, 2%, 1%, or 0.5% of those stereoisomer(s), when compared as a weight % relative to all stereoisomers of alpha tocotrienol hydroquinone combined.

### Topical Cosmetic, Skin Care, and Ophthalmic Compositions

Compositions may be formulated for topical use in a wide variety of cosmetic and skin care compositions, including but not limited to soaps, facial washing cream, facial washing foam, cleansing cream, cleansing milk, cleansing lotion, massage cream, lotion, face lotion, body lotion, skin nourishing lotion, cold cream, moisturizing cream, emulsion, pack, after-shaving cream, sun-screening cream, suntan oil, night cream, night lotion, morning lotion, eye cream, eye essence, cleaning cream, makeup, makeup base, foundation, body shampoo, hair shampoo, hair rinse, hair conditioner, hair-nourishing agent, hair-growing agent, stick pomade, hair cream, hair liquid, hair-setting lotion, hair spray, hair dye, hair bleach, coloring rinse, coloring spray, permanent wave liquid, pressed powder, loose powder, eye shadow, hand cream, lipstick, or a similar product, over-the-counter or prescription topical medicines, etc. They may also be formulated for use in over-the-counter or prescription ophthalmic compositions such as anti-redness eyedrops, contact cleaning solutions, etc. In some embodiments, the stereoisomer(s) described herein are added to a topical composition as a preservative, e.g. to prevent oxidation of one or more components of the composition. In some embodiments, the stereoisomer(s) described herein are added to a topical composition for their beneficial effect on the skin or eye and/or to counteract a pro-oxidant activity of another component in the composition. Without wishing to be bound by any particular theory, the compositions described herein may have an antioxidant effect on animal tissues. Such compositions can be formulated as, e.g., ointments, lotions, creams, foams, gels, sprays, fluids, drops, etc. with standard ingredients as would be known to one skilled in the art. The stereoisomer(s) described herein may be present in the topical composition in any of the amounts or weight percentages described herein.

### Food and Supplement Compositions

The compositions described herein may be added to, or formulated as a part of a food product. The food may be formulated for human use, or for animal use (e.g. a pet food, a livestock food, a laboratory animal food). A composition of the present disclosure can be used in, for example, hospital food, baby food, food products for immunocompromised subjects, or for healthy subjects. In some embodiments, the stereoisomer(s) described herein are added to a food as a nutritional supplement (e.g. a functional food). In some embodiments, the stereoisomer(s) described herein are added to a food for use as a preservative. Without wishing to be bound by any particular theory, a composition may be used as a preservative to preserve freshness, prevent oxidation, and/or to protect natural flavor in the food product, over time, and thus may lead to an increase in shelf-life of the product. A composition may be used as an additive to make the food resistant to high temperature food processing steps, and have low volatility and good solubility in fats and oils. A composition may be useful in fat or oils, for example, vegetable and fish oils, milkfat, poultry fat, and citrus oils. A composition may be used in food goods such as baked goods, packaged foods, cereals, dehydrated potatoes, nuts, noodles, meat products, and egg products. The stereoisomer(s) described herein may be present in the food product in any of the amounts or weight percentages described herein.

The compositions described herein may also be formulated as a nutritional, nutraceutical, dietary, or vitamin supplement, for e.g. humans, pets, or livestock. The stereroisomer(s) described herein may be the sole active ingredient(s), or they may be combined with other nutritional, nutraceutical, or dietary supplements, such as minerals, vitamins, essential fatty acids, etc. They may be formulated for occasional or regular use, such as multiple times a day, daily, every other day, every week, etc. The supplement may be provided in various forms, e.g. pills, capsules, liquid, powders, etc. utilizing standard ingredients as would be known to one skilled in the art. The stereoisomer(s) described herein may be present in the nutritional or vitamin composition in any of the amounts or weight percentages described herein.

### Medical Foods

In some embodiments, medical foods are foods that are specially formulated and intended for the dietary management of a disease that has distinctive nutritional needs that cannot be met by normal diet alone. Such medical foods are labeled for the dietary management of a specific medical disorder, disease or condition for which there are distinctive nutritional requirements, and are intended to be used under medical supervision. In some embodiments, the compositions described herein may be formulated as a medical food, including one or more of dietary carbohydrates, e.g., sugars and starches, dietary fats, dietary proteins, e.g., whey proteins, soy proteins and the like, vitamins, mineral, etc. In some embodiments, the medical food is a nutritionally complete formula. In some embodiments, the medical food is a nutritionally incomplete formula. The stereoisomer(s) described herein may be present in the medical food in any of the amounts or weight percentages described herein; an appropriate amount of the stereoisomer(s) described herein for the particular disease may be determined by one skilled in the art.

### Pharmaceutical Compositions

The compounds described herein may be used in pharmaceutical applications. Non-limiting examples of disease indications for which the compounds and compositions described herein may be used include those described in U.S. Provisional Application No. 62/485,832, titled “METHODS AND COMPOSITIONS FOR TREATMENT OF INFLAMMATION AND OXIDATIVE STRESS,” filed on Apr. 14, 2017, by the same Applicant; U.S. Provisional Application No. 62/488,658, titled “METHODS AND COMPOSITIONS FOR TREATMENT OF INFLAMMATION AND OXIDATIVE STRESS,” filed on Apr. 21, 2017, also by the same Applicant; U.S. Provisional Application No. 62/531,866, titled “METHODS AND COMPOSITIONS FOR TREATMENT OF INFLAMMATION AND OXIDATIVE STRESS,” filed on Jul. 12, 2017, also by the same Applicant; U.S. Provisional Application No. 62/534,649, titled “METHODS AND COMPOSITIONS FOR TREATMENT OF INFLAMMATION AND OXIDATIVE STRESS,” filed on Jul. 19, 2017, also by the same Applicant; U.S. Provisional Application No. 62/572,406, titled “METHODS AND COMPOSITIONS FOR TREATMENT OF INFLAMMATION AND OXIDATIVE STRESS,” filed on Oct. 13, 2017, also by the same Applicant; and PCT Application No. PCT/US2018/27682, titled “METHODS AND COMPOSITIONS FOR TREATMENT OF INFLAMMATION AND OXIDATIVE STRESS,” filed on Apr. 14, 2018, also by the same Applicant, each of which is incorporated by reference herein in its entirety.

The compounds described herein can be formulated as pharmaceutical compositions by formulation with additives such as pharmaceutically acceptable excipients, pharmaceutically acceptable carriers, and pharmaceutically acceptable vehicles. Suitable pharmaceutically acceptable excipients, carriers and vehicles include processing agents and drug delivery modifiers and enhancers, for example, calcium phosphate, magnesium stearate, talc, monosaccharides, disaccharides, starch, gelatin, cellulose, methyl cellulose, sodium carboxymethyl cellulose, dextrose, hydroxypropyl-β-cyclodextrin, polyvinylpyrrolidinone, low melting waxes, ion exchange resins, and the like, as well as combinations of any two or more thereof. Other suitable pharmaceutically acceptable excipients are described in “Remington's Pharmaceutical Sciences,” Mack Pub. Co., N.J. (1991), and “Remington: The Science and Practice of Pharmacy,” Lippincott Williams & Wilkins, Philadelphia, 20th edition (2003) and 21st edition (2005), incorporated herein by reference.

A pharmaceutical composition can comprise a unit dose formulation, where the unit dose is a dose sufficient to have a therapeutic effect. The unit dose may be sufficient as a single dose to have a therapeutic effect. Alternatively, the unit dose may be a dose administered periodically in a course of treatment of a disorder.

Pharmaceutical compositions containing the compounds of this disclosure may be in any form suitable for the intended method of administration, including, for example, a solution, a suspension, or an emulsion. Liquid carriers are typically used in preparing solutions, suspensions, and emulsions. Liquid carriers contemplated for use in the practice of the present disclosure include, for example, water, saline, pharmaceutically acceptable organic solvent(s), pharmaceutically acceptable oils or fats, and the like, as well as mixtures of two or more thereof. The liquid carrier may contain other suitable pharmaceutically acceptable additives such as solubilizers, emulsifiers, nutrients, buffers, preservatives, suspending agents, thickening agents, viscosity regulators, stabilizers, and the like. Suitable organic solvents include, for example, monohydric alcohols, such as ethanol, and polyhydric alcohols, such as glycols. Suitable oils include, for example, soybean oil, sesame oil, coconut oil, olive oil, safflower oil, cottonseed oil, and the like. For parenteral administration, the carrier can also be an oily ester such as ethyl oleate, isopropyl myristate, and the like. Compositions of the present disclosure may also be in the form of microparticles, microcapsules, liposomal encapsulates, and the like, as well as combinations of any two or more thereof.

Time-release or controlled release delivery systems may be used, such as a diffusion controlled matrix system or an erodible system, as described for example in: Lee, “Diffusion-Controlled Matrix Systems”, pp. 155-198 and Ron and Langer, “Erodible Systems”, pp. 199-224, in “Treatise on Controlled Drug Delivery”, A. Kydonieus Ed., Marcel Dekker, Inc., New York. 1992. The matrix may be, for example, a biodegradable material that can degrade spontaneously in situ and in vivo, for example, by hydrolysis or enzymatic cleavage, e.g., by proteases. The delivery system may be, for example, a naturally occurring or synthetic polymer or copolymer, for example, in the form of a hydrogel. Exemplary polymers with cleavable linkages include polyesters, polyorthoesters, polyanhydrides, polysaccharides, poly(phosphoesters), polyamides, polyurethanes, poly(imidocarbonates), and poly(phosphazenes).

The compounds of the disclosure may be administered enterally, orally, parenterally, sublingually, by inhalation (e.g., as mists or sprays), rectally, or topically in dosage unit formulations containing conventional nontoxic pharmaceutically acceptable carriers, adjuvants, and vehicles as desired. For example, suitable modes of administration include oral, subcutaneous, transdermal, transmucosal, iontophoretic, intravenous, intraarterial, intramuscular, intraperitoneal, intranasal (e.g., via nasal mucosa), subdural, rectal, gastrointestinal, and the like, and directly to a specific or affected organ or tissue. For delivery to the central nervous system, spinal and epidural administration, or administration to cerebral ventricles, can be used. Topical administration may also involve the use of transdermal administration such as transdermal patches or iontophoresis devices. The term parenteral as used herein includes subcutaneous injections, intravenous, intramuscular, intrasternal injection, or infusion techniques. The compounds are mixed with pharmaceutically acceptable carriers, adjuvants, and vehicles appropriate for the desired route of administration. Oral administration is a preferred route of administration, and formulations suitable for oral administration are preferred formulations. The compounds described for use herein can be administered in solid form, in liquid form, in aerosol form, or in the form of tablets, pills, powder mixtures, capsules, granules, injectables, creams, solutions, suppositories, enemas, colonic irrigations, emulsions, dispersions, food premixes, and in other suitable forms. The compounds can also be administered in liposome formulations. Additional methods of administration are known in the art.

In some embodiments of this disclosure, especially those embodiments where a formulation is used for injection or other parenteral administration including the routes listed herein, but also including embodiments used for oral, gastric, gastrointestinal, or enteric administration, the formulations and preparations used in the methods of this disclosure are sterile. Sterile pharmaceutical formulations are compounded or manufactured according to pharmaceutical-grade sterilization standards (United States Pharmacopeia Chapters 797, 1072, and 1211; California Business & Professions Code 4127.7; 16 California Code of Regulations 1751, 21 Code of Federal Regulations 211) known to those of skill in the art.

Injectable preparations, for example, sterile injectable aqueous or oleaginous suspensions, may be formulated according to the known art using suitable dispersing or wetting agents and suspending agents. The sterile injectable preparation may also be a sterile injectable solution or suspension in a nontoxic parenterally acceptable diluent or solvent, for example, as a solution in propylene glycol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents.

The compounds of the present disclosure can also be administered in the form of liposomes. As known in the art, liposomes are generally derived from phospholipids or other lipid substances. Liposomes are formed by mono- or multilamellar hydrated liquid crystals that are dispersed in an aqueous medium. Any non-toxic, physiologically acceptable and metabolizable lipid capable of forming liposomes can be used. The present compositions in liposome form can contain, in addition to a compound of the present disclosure, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N. W., p. 33 et seq (1976).

This disclosure also provides articles of manufacture and kits containing the compositions described herein. This disclosure also provides kits comprising a composition as described herein in a suitable container, and instructions for use. In some embodiments, the kit of this disclosure comprises the container described above.

The amount of active ingredient that may be combined with the carrier materials to produce a single dosage form will vary depending upon the host to which the active ingredient is administered and the particular mode of administration. It will be understood, however, that the specific dose level for any particular subject will depend upon a variety of factors including the activity of the specific compound employed, the age, body weight, body area, body mass index (BMI), general health, sex, diet, time of administration, route of administration, rate of excretion, drug combination, and the type, progression, and severity of the particular disease undergoing therapy. The pharmaceutical unit dosage chosen is usually fabricated and administered to provide a defined final concentration of drug in the blood, tissues, organs, or other targeted region of the body. The therapeutically effective amount for a given situation can be readily determined by routine experimentation and is within the skill and judgment of the ordinary clinician.

Examples of dosages which can be used are a therapeutically effective amount within the dosage range of about 0.1 mg/kg to about 300 mg/kg body weight, or within about 1.0 mg/kg to about 100 mg/kg body weight, or within about 1.0 mg/kg to about 50 mg/kg body weight, or within about 1.0 mg/kg to about 30 mg/kg body weight, or within about 1.0 mg/kg to about 10 mg/kg body weight, or within about 10 mg/kg to about 100 mg/kg body weight, or within about 50 mg/kg to about 150 mg/kg body weight, or within about 100 mg/kg to about 200 mg/kg body weight, or within about 150 mg/kg to about 250 mg/kg body weight, or within about 200 mg/kg to about 300 mg/kg body weight, or within about 250 mg/kg to about 300 mg/kg body weight. Compounds of the present disclosure may be administered in a single daily dose, or the total daily dosage may be administered in divided dosage of two, three, or four times daily.

In some or any embodiments, the compounds of this disclosure are administered as the sole active agent(s) in the composition. In some or any embodiments, the compounds of this disclosure are administered as the sole active agent used in the recited method of treatment. In some or any embodiments, the compounds of this disclosure are administered as the sole active agent(s).

In some or any embodiments, the composition of this disclosure is the sole active agent used in the recited method of treatment. In some or any embodiments, the composition of this disclosure is administered as the sole active agent. In some or any embodiments, the composition of this disclosure consists of the recited compound(s) as the sole active agent(s) administered in the method of treatment. In some or any embodiments, the composition of this disclosure consists of the recited compound(s) as the sole active agent(s) administered. In some or any embodiments, the composition of this disclosure is administered as the sole composition containing an active agent(s).

In some or any embodiments, the composition of this disclosure consists of the recited compounds as the sole active agent(s) in the composition. In some or any embodiments, the compounds of this disclosure are present as the sole active agent(s) in the composition.

In some or any embodiments, the compounds of this disclosure are administered as the sole active agent(s) in a therapeutically effective amount or physiologically effective amount in the composition. In some or any embodiments, the compounds of this disclosure are administered as the sole active agent in a therapeutically effective amount or physiologically effective amount used in the recited method of treatment. In some or any embodiments, the compounds of this disclosure are administered as the sole active agent(s) in a therapeutically effective amount or physiologically effective amount.

In some or any embodiments, the composition of this disclosure is the sole active agent used in a therapeutically effective amount or physiologically effective amount in the recited method of treatment. In some or any embodiments, the composition of this disclosure is administered as the sole active agent in a therapeutically effective amount or physiologically effective amount. In some or any embodiments, the composition of this disclosure consists of the recited compound(s) as the sole active agent(s) administered in a therapeutically effective amount or physiologically effective amount in the method of treatment. In some or any embodiments, the composition of this disclosure consists of the recited compound(s) as the sole active agent(s) administered in a therapeutically effective amount or physiologically effective amount. In some or any embodiments, the composition of this disclosure is administered as the sole composition containing an active agent(s) in a therapeutically effective amount or physiologically effective amount.

While the compounds of this disclosure can be administered as the sole active agent, they can also be used in combination with one or more other agents used in the treatment of the particular disorder. When additional active agents are used in combination with the compounds of the present disclosure, the additional active agents may generally be employed in therapeutic amounts as indicated in the Physicians' Desk Reference (PDR) 53rd Edition (1999), or such therapeutically useful amounts as would be known to one of ordinary skill in the art.

The compounds of this disclosure and the other therapeutically active agents can be administered at the recommended maximum clinical dosage or at lower doses. Dosage levels of the active compounds in the compositions of this disclosure may be varied so as to obtain a desired therapeutic response depending on the route of administration, severity of the disease and the response of the patient. When administered in combination with other therapeutic agents, the therapeutic agents can be formulated as separate compositions that are given at the same time or different times, or the therapeutic agents can be given as a single composition.

Non-limiting embodiments of this disclosure are listed below.
1. A composition comprising a synthetic, non-plant based, substantially pure stereoisomer of an alpha, beta, gamma, or delta tocopherol or tocotrienol, or the quinone or hydroquinone form thereof, or a metabolite thereof.
2. The composition of embodiment 1, wherein the stereoisomer is selected from the group consisting of: (R,R,R)-alpha-tocopherol, (R,R,R)-beta-tocopherol, (R,R,R)-gamma-tocopherol, (R,R,R)-delta-tocopherol, (R,E,E)-alpha-tocotrienol, (R,E,E)-beta-tocotrienol, (R,E,E)-gamma-tocotrienol, (R,E,E)-delta-tocotrienol, (R,R,R)-alpha-tocopherol quinone, (R,R,R)-beta-tocopherol quinone, (R,R,R)-gamma-tocopherol quinone, (R,R,R)-delta-tocopherol quinone, (R,E,E)-alpha-tocotrienol quinone, (R,E,E)-beta-tocotrienol quinone, (R,E,E)-gamma-tocotrienol quinone, (R,E,E)-delta-tocotrienol quinone, (R,R,R)-alpha-tocopherol hydroquinone, (R,R,R)-beta-tocopherol hydroquinone, (R,R,R)-gamma-tocopherol hydroquinone, (R,R,R)-delta-tocopherol hydroquinone, (R,E,E)-alpha-tocotrienol hydroquinone, (R,E,E)-beta-tocotrienol hydroquinone, (R,E,E)-gamma-tocotrienol hydroquinone, (R,E,E)-delta-tocotrienol hydroquinone, and metabolites thereof.
3. The composition of embodiment 1, wherein the stereoisomer is (R,R,R)-alpha-tocopherol.
4. The composition of embodiment 1, wherein the stereoisomer is (R,R,R)-beta-tocopherol.
5. The composition of embodiment 1, wherein the stereoisomer is (R,R,R)-gamma-tocopherol.
6. The composition of embodiment 1, wherein the stereoisomer is (R,R,R)-delta-tocopherol.
7. The composition of embodiment 1, wherein the stereoisomer is (R,E,E)-alpha-tocotrienol.
8. The composition of embodiment 1, wherein the stereoisomer is (R,E,E)-beta-tocotrienol.
9. The composition of embodiment 1, wherein the stereoisomer is (R,E,E)-gamma-tocotrienol.
10. The composition of embodiment 1, wherein the stereoisomer is (R,E,E)-delta-tocotrienol.
11. The composition of embodiment 1, wherein the stereoisomer is (R,R,R)-alpha-tocopherol quinone.
12. The composition of embodiment 1, wherein the stereoisomer is (R,R,R)-beta-tocopherol quinone.
13. The composition of embodiment 1, wherein the stereoisomer is (R,R,R)-gamma-tocopherol quinone.
14. The composition of embodiment 1, wherein the stereoisomer is (R,R,R)-delta-tocopherol quinone.
15. The composition of embodiment 1, wherein the stereoisomer is (R,E,E)-alpha-tocotrienol quinone.
16. The composition of embodiment 1, wherein the stereoisomer is (R,E,E)-beta-tocotrienol quinone.
17. The composition of embodiment 1, wherein the stereoisomer is (R,E,E)-gamma-tocotrienol quinone.
18. The composition of embodiment 1, wherein the stereoisomer is (R,E,E)-delta-tocotrienol quinone.
19. The composition of embodiment 1, wherein the stereoisomer is (R,R,R)-alpha-tocopherol hydroquinone.
20. The composition of embodiment 1, wherein the stereoisomer is (R,R,R)-beta-tocopherol hydroquinone.
21. The composition of embodiment 1, wherein the stereoisomer is (R,R,R)-gamma-tocopherol hydroquinone.
22. The composition of embodiment 1, wherein the stereoisomer is (R,R,R)-delta-tocopherol hydroquinone.
23. The composition of embodiment 1, wherein the stereoisomer is (R,E,E)-alpha-tocotrienol hydroquinone.
24. The composition of embodiment 1, wherein the stereoisomer is (R,E,E)-beta-tocotrienol hydroquinone.
25. The composition of embodiment 1, wherein the stereoisomer is (R,E,E)-gamma-tocotrienol hydroquinone.
26. The composition of embodiment 1, wherein the stereoisomer is (R,E,E)-delta-tocotrienol hydroquinone.
27. The composition of any one of embodiments 1-26, wherein the substantially pure stereoisomer comprises at least about 0.001% by weight of the composition.
28. The composition of any one of embodiments 1-26, wherein the substantially pure stereoisomer comprises at least about 0.01% by weight of the composition.
29. The composition of any one of embodiments 1-26, wherein the substantially pure stereoisomer comprises at least about 0.1% by weight of the composition.
30. The composition of any one of embodiments 1-26, wherein the substantially pure stereoisomer comprises at least about 1.0% by weight of the composition.
31. The composition of any one of embodiments 1-26, wherein the substantially pure stereoisomer comprises at least about 10.0% by weight of the composition.
32. A composition comprising one or more synthetic, non-plant based, substantially pure stereoisomers of:
   (a) a tocopherol selected from the group consisting of: alpha-tocopherol, beta-tocopherol, gamma-tocopherol, and delta-tocopherol, wherein the one or more stereoisomers of the tocopherol comprise an asymmetric carbon at the 2-position of the chromanol ring, wherein the 2-position asymmetric carbon has an R configuration; or a metabolite thereof; or
   (b) a tocopherol quinone selected from the group consisting of alpha-tocopherol quinone, beta-tocopherol quinone, gamma-tocopherol quinone, and delta-tocopherol quinone, wherein the one or more stereoisomers of the tocopherol quinone comprise an asymmetric carbon at the 3-position of the tail group, wherein the 3-position of the tail group has an R configuration; or a metabolite thereof; or
   (c) a tocopherol hydroquinone selected from the group consisting of alpha-tocopherol hydroquinone, beta-tocopherol hydroquinone, gamma-tocopherol hydroquinone, and delta-tocopherol hydroquinone, wherein the one or more stereoisomers of the tocopherol quinone comprise an asymmetric carbon at the 3-position of the tail group, wherein the 3-position of the tail group has an R configuration; or a metabolite thereof.
33. A composition comprising one or more synthetic, non-plant based, substantially pure stereoisomers of:
   (a) a tocotrienol selected from the group consisting of: alpha-tocotrienol, beta-tocotrienol, gamma-tocotrienol, and delta-tocotrienol, wherein the one or more stereoisomers of the tocotrienol comprise an asymmetric carbon at the 2-position of the chromanol ring, wherein the 2-position asymmetric carbon has an R configuration; or a metabolite thereof; or
   (b) a tocotrienol quinone selected from the group consisting of alpha-tocotrienol quinone, beta-tocotrienol quinone, gamma-tocotrienol quinone, and delta-tocotrienol quinone, wherein the one or more stereoisomers of the tocotrienol quinone comprise an asymmetric carbon at the 3-position of the tail group, wherein the 3-position of the tail group has an R configuration; or a metabolite thereof; or
   (c) a tocotrienol hydroquinone selected from the group consisting of alpha-tocotrienol hydroquinone, beta-tocotrienol hydroquinone, gamma-tocotrienol hydroquinone, and delta-tocopheral hydroquinone, wherein the one or more stereoisomers of the tocotrienol hydroquinone comprise an asymmetric carbon at the 3-position of the tail group, wherein the 3-position of the tail group has an R configuration; or a metabolite thereof.
34. The composition of embodiment 33, wherein the one or more stereoisomers are alpha-tocopherol stereoisomers.
35. The composition of embodiment 33, wherein the one or more stereoisomers are beta-tocopherol stereoisomers.
36. The composition of embodiment 33, wherein the one or more stereoisomers are gamma-tocopherol stereoisomers.
37. The composition of embodiment 33, wherein the one or more stereoisomers are delta-tocopherol stereoisomers.
38. The composition of embodiment 33, wherein the one or more stereoisomers are alpha-tocopherol quinone stereoisomers.
39. The composition of embodiment 33, wherein the one or more stereoisomers are beta-tocopherol quinone stereoisomers.
40. The composition of embodiment 33, wherein the one or more stereoisomers are gamma-tocopherol quinone stereoisomers.
41. The composition of embodiment 33, wherein the one or more stereoisomers are delta-tocopherol quinone stereoisomers.
42. The composition of embodiment 33, wherein the one or more stereoisomers are alpha-tocopherol hydroquinone stereoisomers.
43. The composition of embodiment 33, wherein the one or more stereoisomers are beta-tocopherol hydroquinone stereoisomers.
44. The composition of embodiment 33, wherein the one or more stereoisomers are gamma-tocopherol hydroquinone stereoisomers.
45. The composition of embodiment 33, wherein the one or more stereoisomers are delta-tocopherol hydroquinone stereoisomers.
46. The composition of any one of embodiments 33-45, wherein the substantially pure stereoisomer or stereoisomers comprise at least about 0.001% by weight of the composition.
47. The composition of any one of embodiments 33-45, wherein the substantially pure stereoisomer or stereoisomers comprise at least about 0.01% by weight of the composition.
48. The composition of any one of embodiments 33-45, wherein the substantially pure stereoisomer or stereoisomers comprise at least about 0.1% by weight of the composition.
49. The composition of any one of embodiments 33-45, wherein the substantially pure stereoisomer or stereoisomers comprise at least about 1.0% by weight of the composition.
50. The composition of any one of embodiments 33-45, wherein the substantially pure stereoisomer or stereoisomers comprise at least about 10.0% by weight of the composition.
51. The composition of any one of embodiments 1-50, wherein the purity is at least about 90%.
52. The composition of any one of embodiments 1-50, wherein the purity is at least about 95%.
53. The composition of any one of embodiments 1-50, wherein the purity is at least about 98%.
54. The composition of any one of embodiments 1-50, wherein the purity is at least about 99%.
55. The composition of any one of embodiments 1-50, wherein the purity is at least about 99.5%.
56. A composition comprising one or more synthetic, non-plant based stereoisomers of a compound selected from the group consisting of alpha, beta, gamma, and delta tocopherol or tocotrienol, and the quinone and hydroquinone forms thereof, wherein the synthetic, non-plant based stereoisomers have an activity that is at least about 5% greater than an equimolar mixture of all diastereomers of the compound.
57. A composition comprising one or more synthetic, non-plant based stereoisomers of a compound selected from the group consisting of alpha, beta, gamma, and delta tocopherol or tocotrienol, and the quinone and hydroquinone forms thereof;
   wherein the one or more synthetic, non-plant based stereoisomers of the tocopherol or tocotrienol comprise an asymmetric carbon at the 2-position of the chromanol ring, wherein the 2-position asymmetric carbon has an R configuration;
   wherein the one or more synthetic, non-plant based stereoisomers of the tocopherol or tocotrienol quinone or hydroquinone comprise an asymmetric carbon at the 3-position of the tail group, wherein the 3-position of the tail group has an R configuration; and
   wherein the synthetic, non-plant based stereoisomers have an activity that is at least about 5% greater than an equimolar mixture of the corresponding R and S isomers of the compound.
58. A composition comprising a synthetic, non-plant based alpha-tocopherol, wherein the composition has an alpha-tocopherol activity that is equal to or greater than about 1.1 IU per mg of synthetic, non-plant based alpha-tocopherol.
59. The composition of embodiment 58, wherein the activity is equal to or greater than about 1.4 IU per mg of synthetic, non-plant based alpha-tocopherol.
60. The composition of any one of embodiments 58 or 59, wherein the synthetic, non-plant based alpha-tocopherol comprises a stereoisomer of alpha-tocopherol, wherein the stereoisomer comprises an asymmetric carbon at the 2-position of the chromanol ring, wherein the asymmetric carbon has an R configuration.
61. The composition of embodiment 60, wherein the stereoisomer comprises three asymmetric carbons, wherein each asymmetric carbon has an R configuration.
62. The composition of embodiment 61, wherein the stereoisomer is substantially pure.
63. A composition comprising a synthetic, non-plant based alpha-tocopherol quinone, wherein the composition has a baseline activity in a Sulfide:quinone oxidoreductase colorimetric assay that is at least about 5% higher than a baseline activity of a composition that comprises a mixture of RRR- and SRR-alpha-tocopherol quinones with a diastereomeric ratio of 1:1 in the assay, wherein each composition has an equivalent amount of RRR-alpha-tocopherol quinone.
64. The composition of any one of embodiments 1-63, wherein the composition is a food, a functional food, a medical food, a pharmaceutical formulation, a nutritional supplement, a nutraceutical supplement, a vitamin supplement, a cosmetic formulation, a skin care formulation, or an ophthalmic formulation.
65. The composition of any one of embodiments 1-64, wherein the composition is formulated for human use.
66. The composition of any one of embodiments 1-64, wherein the composition is formulated for animal use.
67. The composition of any one of embodiments 1-66, wherein the composition is formulated for enteral, oral, parenteral, sublingual, inhalation, or rectal use.
68. The composition of any one of embodiments 1-66, wherein the composition is formulated for topical use.
69. The composition of any one of embodiments 1-66, wherein the composition is formulated for oral use.
70. A method of treating an individual, comprising: administering to a subject in need thereof a therapeutically effective amount or a physiologically effective amount of a composition according to any one of embodiments 1-69.
71. A method for converting a tocopherol or tocotrienol quinone to its corresponding chroman, comprising mixing the quinone with HCOOH and Pd/C.
72. The method of embodiment 71, wherein the mixture is heated.
73. The method of claim 71 or 72, wherein alpha-tocopherol quinone is converted to alpha-tocopherol.
74. The method of claim 71 or 72, wherein alpha-tocotrienol quinone is converted to alpha-tocotrienol.

Provided herein is a composition comprising a synthetic, non-plant based, substantially pure stereoisomer of an alpha, beta, gamma, or delta tocopherol or tocotrienol, or the quinone or hydroquinone form thereof, or a metabolite thereof. In some or any embodiments, provided is the composition wherein the stereoisomer is (R,R,R)-alpha-tocopherol. In some or any embodiments, provided is the composition wherein the stereoisomer is (R,E,E)-alpha-tocotrienol. In some or any embodiments, provided is the composition, wherein the stereoisomer is (R,R,R)-alpha-tocopherol quinone. In some or any embodiments, provided is the composition wherein the stereoisomer is (R,E,E)-alpha-tocotrienol quinone. In some or any embodiments, provided is the composition wherein the stereoisomer is (R,R,R)-alpha-tocopherol hydroquinone. In some or any embodiments, provided is the composition wherein the stereoisomer is (R,E,E)-alpha-tocotrienol hydroquinone. In some or any embodiments, provided is the composition wherein the substantially pure stereoisomer comprises at least about 95%.

Provided herein is a composition comprising one or more synthetic, non-plant based, substantially pure stereoisomers of:
(a) a tocopherol selected from the group consisting of: alpha-tocopherol, beta-tocopherol, gamma-tocopherol, and delta-tocopherol, wherein the one or more stereoisomers of the tocopherol comprise an asymmetric carbon at the 2-position of the chromanol ring, wherein the 2-position asymmetric carbon has an R configuration; or a metabolite thereof; or
(b) a tocopherol quinone selected from the group consisting of alpha-tocopherol quinone, beta-tocopherol quinone, gamma-tocopherol quinone, and delta-tocopherol quinone, wherein the one or more stereoisomers of the tocopherol quinone comprise an asymmetric carbon at the 3-position of the tail group, wherein the 3-position of the tail group has an R configuration; or a metabolite thereof; or
(c) a tocopherol hydroquinone selected from the group consisting of alpha-tocopherol hydroquinone, beta-tocopherol hydroquinone, gamma-tocopherol hydroquinone, and delta-tocopherol hydroquinone, wherein the one or more stereoisomers of the tocopherol hydroquinone comprise an asymmetric carbon at the 3-position of the tail group, wherein the 3-position of the tail group has an R configuration; or a metabolite thereof.

In some or any embodiments, provided is the composition wherein the one or more stereoisomers are alpha-tocopherol stereoisomers. In some or any embodiments, provided is the composition wherein the one or more stereoisomers are alpha-tocopherol quinone stereoisomers. In some or any embodiments, provided is the composition wherein the one or more stereoisomers are alpha-tocopherol hydroquinone stereoisomers. In some or any embodiments, provided is the composition wherein the substantially pure stereoisomer is at least about 95%. In some or any embodiments, provided is the composition wherein the composition is formulated for human use. In some or any embodiments, provided is the composition wherein the composition is formulated for animal use.

Provided is a method of treating an individual, comprising: administering to a subject in need thereof a therapeutically effective amount or a physiologically effective amount of a composition according to any embodiment disclosed herein.

Provided is a method for converting a tocopherol quinone or tocotrienol quinone to its corresponding chroman, comprising mixing the quinone with HCOOH and Pd/C. In some or any embodiments, provided is the method wherein the mixture is heated.

Provided is a method for synthesizing (R,E,E)-α-tocotrienol quinone or (S,E,E)-α-tocotrienol quinone comprising

(a) treating the compound of Formula IXa or (IXb):

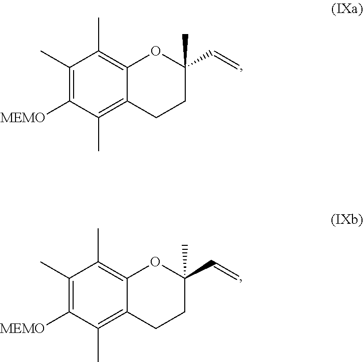

first with an organoborane in a first polar aprotic solvent; followed by a palladium catalyst, a compound of Formula V:

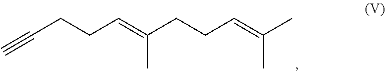

and a phosphate base in a second polar aprotic solvent and water to form a compound of Formula Xa or Xb, respectively:

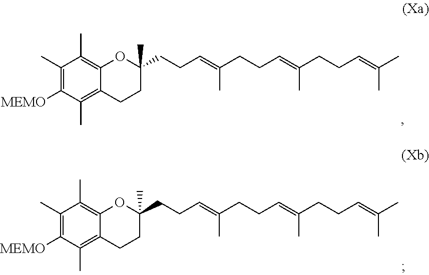

and
(b) treating the compound of Formula Xa or Xb with an oxidizing agent and a carbonate base in a mixture of a third polar aprotic solvent and water, to form (R,E,E)-α-tocotrienol quinone or (S,E,E)-α-tocotrienol quinone, respectively. In some or any embodiments, provided is a method wherein the organoborane is 9-BBN, the palladium catalyst is PdCl₂(dppf), the phosphate base is potassium phosphate. In some or any embodiments, provided is a method wherein the first polar aprotic solvent is selected from the group consisting of DMF and THF, the second polar aprotic solvent is selected from the group consisting of DMF and THF, and the third polar aprotic solvent is selected from the group consisting of isopropyl acetate.

In some or any embodiments, provided is a method for synthesizing (R,E,E)-α-tocotrienol quinone comprising

(a) treating the compound of Formula IXa:

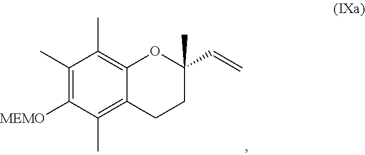

first with an organoborane in a first polar aprotic solvent; followed by a palladium catalyst, a compound of Formula V:

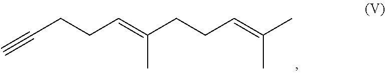

and a phosphate base in a second polar aprotic solvent and water to form a compound of Formula Xa:

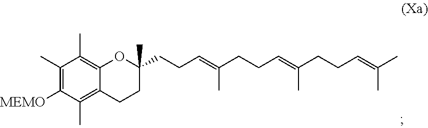

and
(b) treating the compound of Formula Xa with an oxidizing agent and a carbonate base in a mixture of a third polar aprotic solvent and water, to form (R,E,E)-α-tocotrienol quinone. In some or any embodiments, provided is a method wherein the organoborane is 9-BBN, the palladium catalyst is PdCl₂(dppf), the phosphate base is potassium phosphate. In some or any embodiments, provided is a method wherein the first polar aprotic solvent is selected from the group consisting of DMF and THF, the second polar aprotic solvent is selected from the group consisting of DMF and THF, and the third polar aprotic solvent is selected from the group consisting of isopropyl acetate.

In some or any embodiments, provided is a method for synthesizing ((S,E,E)-α-tocotrienol quinone comprising

(a) treating the compound of Formula (IXb):

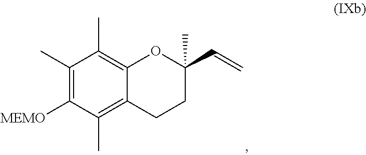

first with an organoborane in a first polar aprotic solvent; followed by a palladium catalyst, a compound of Formula V:

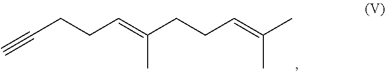

and a phosphate base in a second polar aprotic solvent and water to form a compound of Formula Xb:

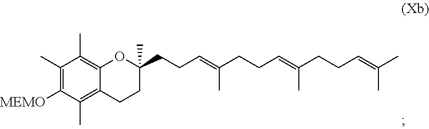

and
(b) treating the compound of Formula Xb with an oxidizing agent and a carbonate base in a mixture of a third polar aprotic solvent and water, to form (S,E,E)-α-tocotrienol quinone, respectively. In some or any embodiments, provided is a method wherein the organoborane is 9-BBN, the palladium catalyst is PdCl₂(dppf), the phosphate base is potassium phosphate. In some or any embodiments, provided is a method wherein the first polar aprotic solvent is selected from the group consisting of DMF and THF, the second polar aprotic solvent is selected from the group consisting of DMF and THF, and the third polar aprotic solvent is selected from the group consisting of isopropyl acetate.

### Methods of Synthesis

Compounds (including single stereoisomers and mixture of stereoisomers) of the present disclosure can be prepared and synthesized by a method described herein, or by similar or alternative methods that would be apparent to one skilled in the art, in view of the disclosure provided herein.

The quinone form of a compound can be reduced to the dihydroquinone form with reducing agents such as sodium dithionite. The hydroquinone form can be oxidized to the quinone form with oxidizing agents such as ceric ammonium nitrate or ferric chloride. The quinone and hydroquinone forms are also readily converted electrochemically, as is well known in the art. See, e.g., Section 33.4 of Streitweiser & Heathcock, Introduction to Organic Chemistry, New York: Macmillan, 1976.

After synthesis of a compound, the compound may be isolated from the reaction mixture by filtration, extraction, centrifugation, solubilization, concentration, washing, adsorption, purification, or a combination thereof.

Further purification of a compound of the present disclosure may comprise chromatography, fractionation, crystallization, or a combination thereof. In some embodiments, a compound may be purified by High Performance Liquid Chromatography (HPLC), column chromatography, gas chromatography, supercritical fluid chromatography, ion exchange chromatography, or size-exclusion chromatography.

When desired, the various isomers of the compounds of the present disclosure, if present, may be resolved by various methods, for example, through the formation of diastereoisomeric salts or complexes which may be separated, for example, by crystallization; via formation of diasteroisomeric derivatives which may be separated, for example, by crystallization, gas-liquid or liquid chromatography; selective reaction of one enantiomer with an enantiospecific reagent, for example, enzymatic oxidation or reduction, followed by separation of the modified and unmodified enantiomers; or gas-liquid or liquid chromatography in a chiral environment, for example, on a chiral support, such as silica with a bound chiral ligand or in the presence of a chiral solvent. Alternatively, a specific enantiomer may be synthesized by asymmetric synthesis using optically active reagents, substrates, catalysts or solvents, or by converting one enantiomer to the other by asymmetric transformation. In some embodiments, a composition enriched in one or more of the desired stereoisomers is produced without chromatography techniques (e.g., without using chromatography to separate the desired stereoisomer(s) from the non-desired stereoisomer(s)). As a non-limiting example, a composition enriched in RRR-alpha tocopherol is produced without using chromatography to separate the RRR-alpha tocopherol from other alpha tocopherol stereoisomers (i.e., enantiomers and/or diastereomers).

In some embodiments, the purity of a compound may be measured by a chromatographic method or a spectroscopic method, including, but not limited to, high performance liquid chromatography (HPLC), mass spectrometry, high resolution mass spectrometry, nuclear magnetic resonance spectroscopy, infrared spectroscopy or Raman spectroscopy.

Methods for preparing the composition of the present disclosure are not limited to those described in the examples, and appropriate alterations and modifications can be added to these methods.

### EXAMPLES

The following examples are given for the purpose of illustrating various embodiments of the present disclosure and are not meant to limit the present disclosure in any fashion. The present examples; along with the methods described herein are presently representative of preferred cases; are exemplary; and are not intended as limitations on the scope of the present disclosure. Changes therein and other uses which are encompassed within the spirit of the present disclosure as defined by the scope of the claims will occur to those skilled in the art.

Abbreviations:

| | |
|---|---|
| 9-BBN | 9-Borabicyclo[3.3.1]nonane |
| CAN | Ceric ammonium nitrate |
| Cp₂ZrCl₂ | Zirconocene dichloride |
| DIPEA | N,N-Diisopropylethylamine |
| DMF | Dimethylformamide |
| DMPI | Dess Martin Periodinane |
| dppf | 1,1′-Bis(diphenylphosphino)ferrocene |
| IPA | isopropyl alcohol |
| i-PrOAc | Isopropyl acetate |
| LAH | Lithium aluminium hydride |
| MEMCl | 2-Methoxyethoxymethyl chloride |
| TBAF | Tetra-n-butylammonium fluoride |
| THF | tetrahydrofuran |
| TMS- | Trimethylsilyl |

### Example 1: Synthesis of (R,E,E)-Alpha Tocotrienol Quinone

(R,E,E)-Alpha tocotrienol quinone is synthesized according to the following scheme:

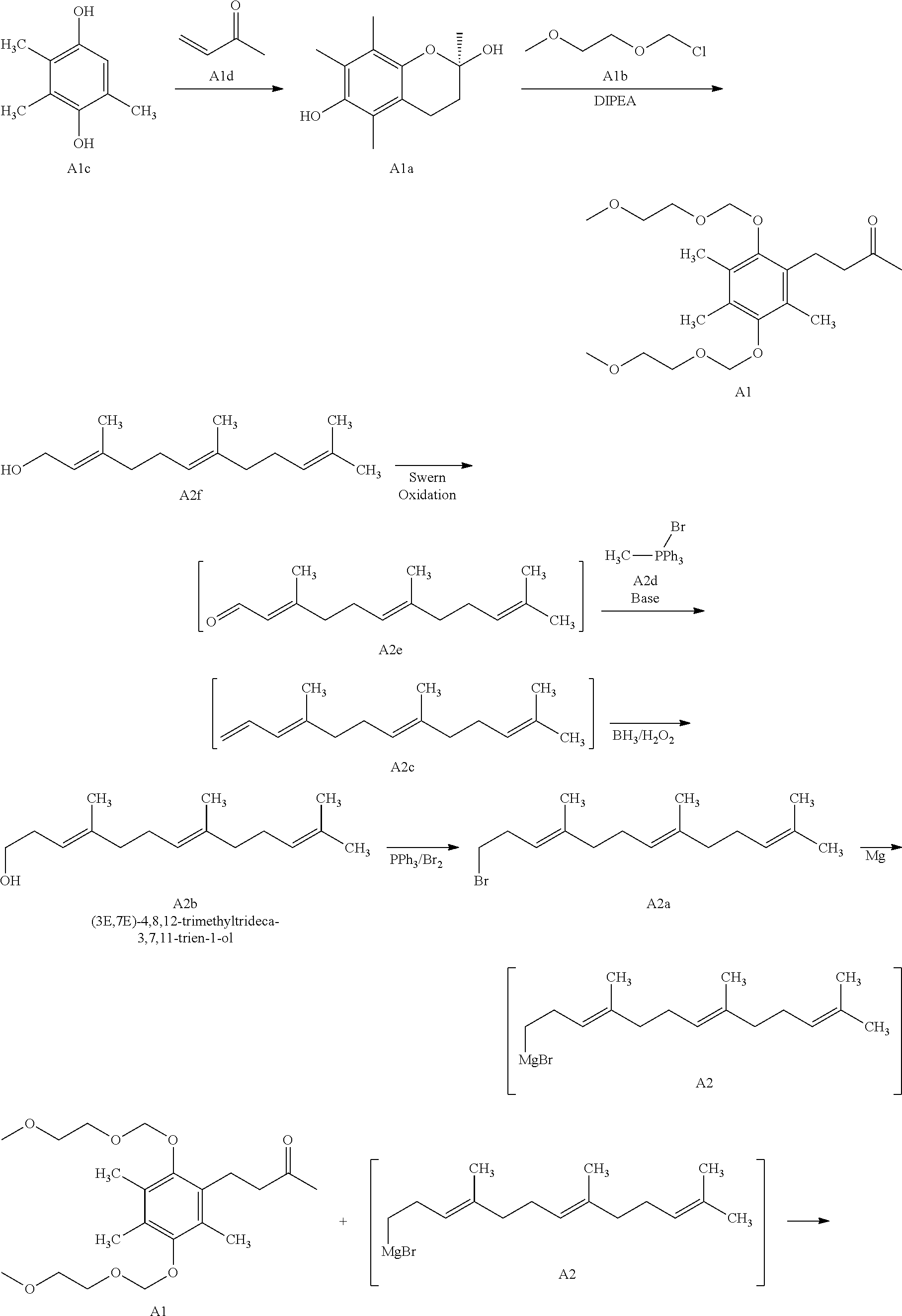

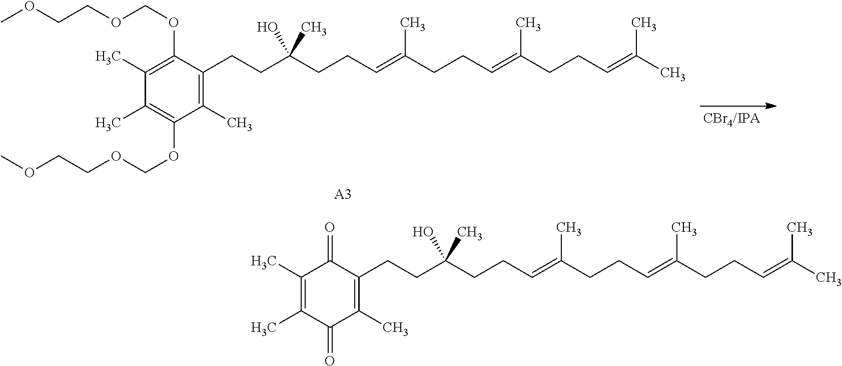

A3 can be prepared by stereoselective Grignard synthesis using A1 and A2 in the presence of L1 or L2:

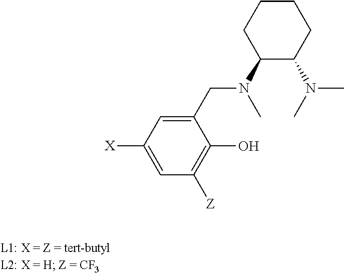

as described, for example, in Bieszczad and Gilheany, Angew. Chem. Int. Ed. 2017, 56, 4272-4276 (and Supporting Information).

### Example 2: Synthesis of (R,E,E)-Alpha-Tocotrienol Quinone

(R,E,E)-Alpha tocotrienol quinone is synthesized according to the following scheme:

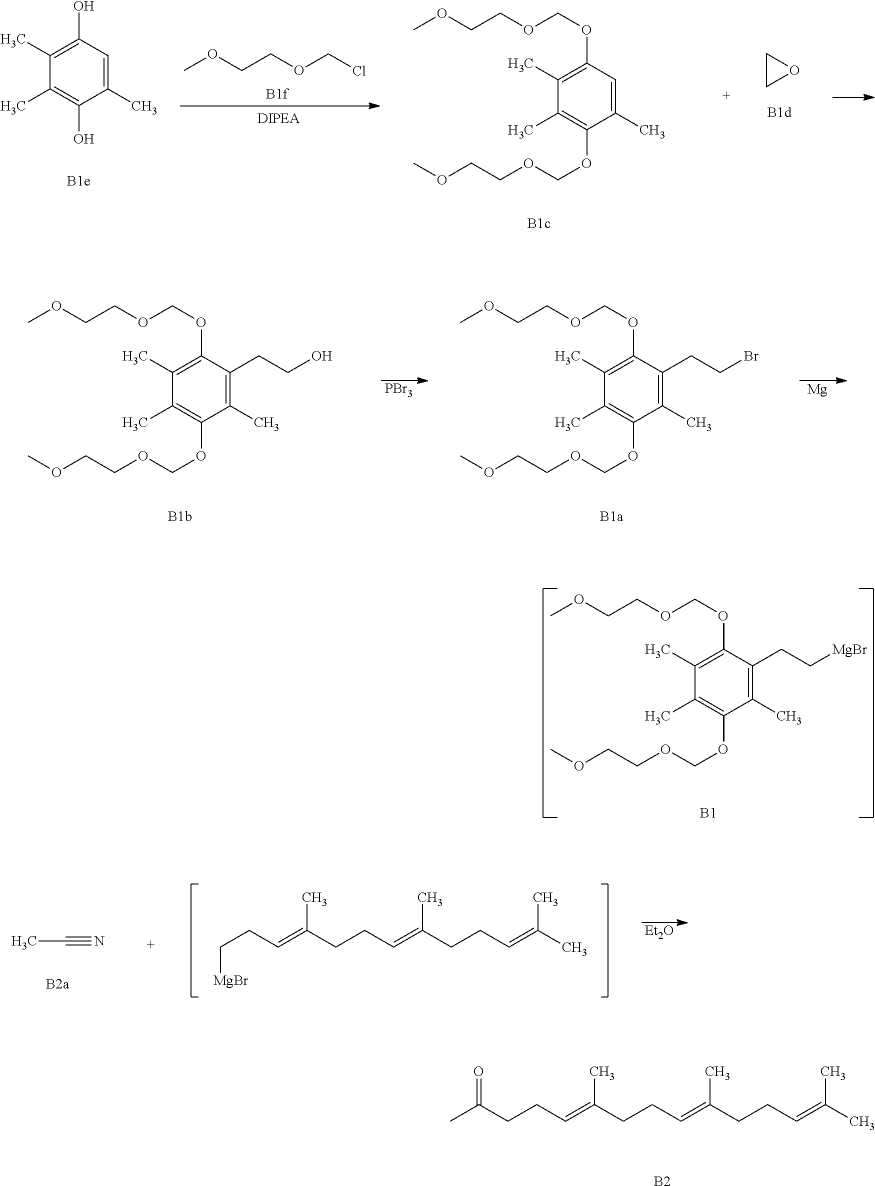

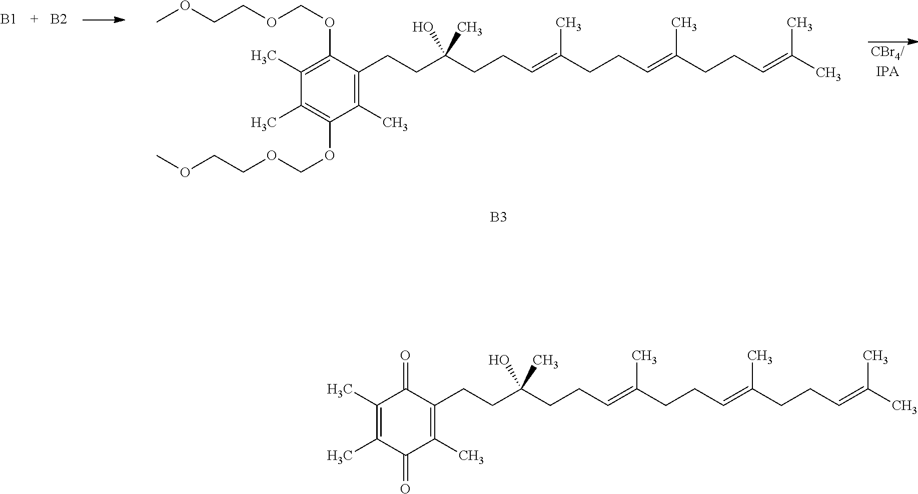

B3 can be prepared by stereoselective Grignard synthesis using B1 and B2 in the presence of L1 or L2:

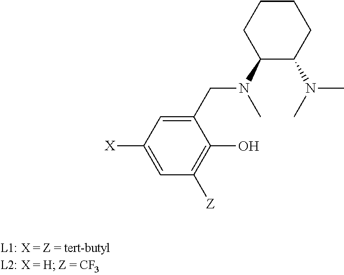

as described, for example, in Bieszczad and Gilheany, Angew. Chem. Int. Ed. 2017, 56, 4272-4276 (and Supporting Information).

### Example 3: Synthesis of (R,R,R)-Alpha Tocopherol Quinone

(R,R,R)-Alpha Tocopherol Quinone is synthesized in a similar manner to Examples 1 or 2, wherein compound A2f is replaced with

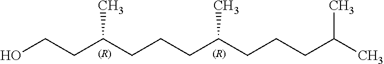

(CAS Reg. No. 54154-26-6), which is commercially available from Angene Chemical (Catalog No. AGN-PC-OSGMB9) or can be prepared using procedures known to one of ordinary skill in the art, including those in Matsueda et al. Tetrahedron Letters 2015, 56(23), 3346-3348; or by following Scheme 2 in Bieszczad et al. Angew. Chem. Int. Ed. 2017, 56, 4272-4276, followed by oxidation of (R,R,R)-alpha tocopherol to (R,R,R)-alpha tocopherol quinone, using conditions described herein.

### Example 4: Synthesis of (R,E,E)-Beta-, Gamma-, and Delta-Tocotrienol Quinones and (R,R,R)- Beta-, Gamma-, and Delta-Tocopherol Quinones

(R,E,E)-Beta-, gamma-, and delta-tocotrienol quinones and (R,R,R)-Beta-, gamma-, and delta-tocopherol quinones are synthesized in a similar manner to Examples 1-3, wherein compounds A1c and B1e are replaced with

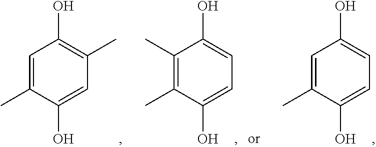

respectively.

### Example 5: Conversion of Alpha Tocopherol Quinone to Alpha Tocopherol (Chroman)

(R,R,R)-Alpha Tocopherol (Chroman) was synthesized from (R,R,R)-Alpha Tocopherol Quinone according to the following scheme:

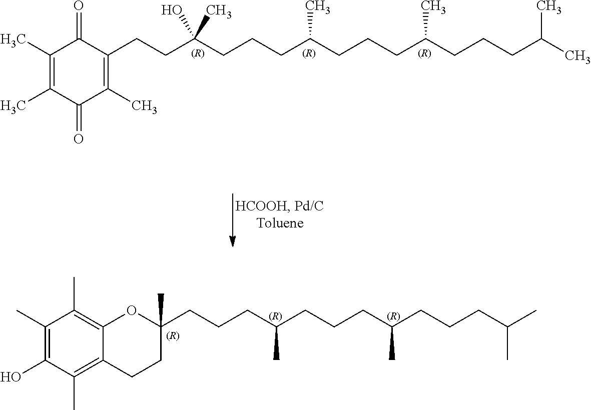

Into a 20 mL scintillation vial and stir bar was added 0.5 g (R,R,R)-α-tocopherol quinone (1.12 mmol), toluene (5 mL), Pd/C (5% wt Pd, ˜30 mg), and HCOOH (338 μL, 413 mg, 8.95 mmol), and the mixture was heated to 60° C. for 21 h. The reaction mixture was filtered through a PE filter and the filter rinsed with iPrOAc (5 mL). The combined organics were washed with 1 M NaHCO₃ (10 mL), dried over anhydrous Na₂SO₄, and concentrated to a brown oil. Purification by flash chromatography (SiO₂) returned 387.4 mg of putative (R,R,R)-α-tocopherol. UV conformed to expected. Purity 95% a/a UPLC. ¹H NMR (C₆D₆) ϵ 3.73 (s, 1H), 3.40 (m, 2H), 2.29 (s, 3H), 1.99 (s, 3H), 1.97 (s, 3H), 1.64-1.32 (m, 20H), 1.21 (s, 3H), 1.13-1.12 (m, 6H), 0.95-0.91 (m, 12 H). ¹³C NMR (C₆D₆) δ 146.0, 145.5, 122.7, 121.4, 118.7, 117.4, 74.4, 40.2, 39.8, 38.0, 37.9, 37.8, 33.3, 33.2, 32.1, 28.4, 25.3, 25.0, 24.0, 23.0, 22.9, 21.5, 21.1, 20.0, 19.9, 12.3, 11.4.

### Example 6: Conversion of Alpha Tocotrienol Quinone to Alpha Tocotrienol (Chroman)

(R,E,E)-Alpha Tocotrienol (Chroman) was synthesized from (R,E,E)-Alpha Tocotrienol Quinone according to the following scheme, in a manner similar to that described in Example 5, except the reaction was performed at room temperature rather than at 60° C.:

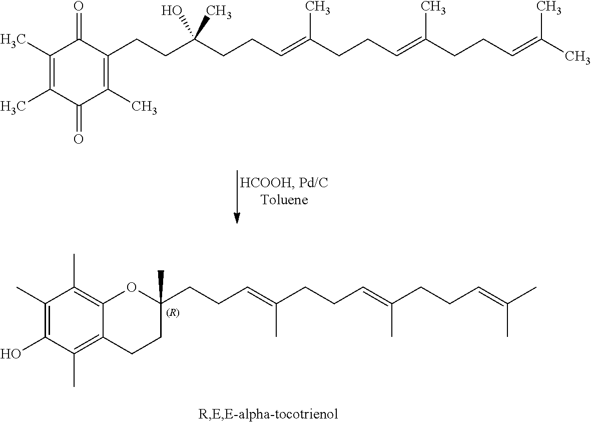

### Example 7: Conversion of (R,E,E)-Alpha Tocotrienol Quinone to (R,E,E)-Alpha Tocotrienol Hydroquinone

(R,E,E)-Alpha Tocotrienol Quinone is converted to the dihydroquinone form with sodium dithionite or hydrogen and palladium catalyst.

### Example 8

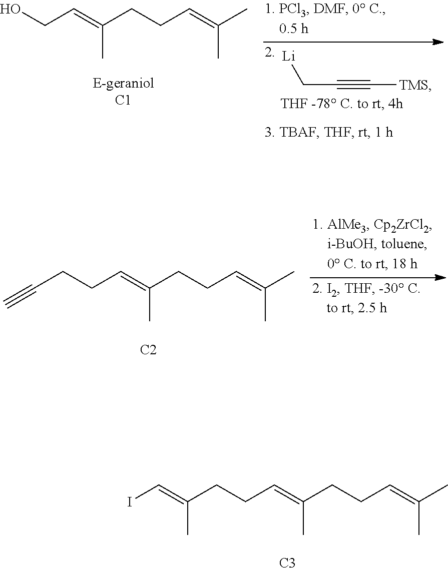

Synthesis of Geranyl Chloride (C2) From Geraniol (C1) For Use in Examples 8a-8c

DMF (48-52 mL) in a room temperature water bath was treated with PCl₃ (7.5 mL, 85.6 mmol) over 2-3 minutes with slow stirring. The stirring was suspended after 1-2 min and the mixture allowed to stand for 2.5 h. A clear solid contained within a clear, light brown liquid was broken up by resumption of stirring, and geraniol (22.75 mL, 20.0 g, 129.7 mmol) in DMF (25 mL) was added over 10 minutes, stirred for 2.25 h and the cloudy orange solution treated with H₂O (100 mL). After 0.5 h, the exotherm subsided, pentane was added, stirred and the organic layer separated. The aqueous layers were washed with pentane (2×50 mL) and the combined organics washed with 10 wt % NaCl (2×50 mL), NaHCO₃ (1×20 mL, post wash pH=9) and 20 wt % NaCl (20 mL). The pentane phase was dried over Na₂SO₄ and concentrated to a clear yellow oil (16.79 g, 75.3%). ¹H NMR showed ˜9% Sn2′ addition product, 2.5 wt % pentane. HPLC, 82% a/a purity with 12.3% Sn2′, 5% geraniol and 1% byproduct. ¹H NMR (CDCl_{3, 400} MHz) δ 5.45 (tq, J=8, 1.2 Hz, 1H), 5.08 (m, 1H), 4.09 (d, J=8 Hz, 2H), 2.08 (m, 4H), 1.72 (d, J=1.2 Hz, 3H), 1.68 (d, J=0.8 Hz, 3H), 1.61 (s, 3H).

Synthesis of Farnesyl Alkyne (C2) For Use in Examples 8a-8c

An N₂-purged flask was treated with THF (130 mL) and charged with TMS-propyne (4.4 mL, 3.33 g, 29.7 mmol), cooled to −78° C. and treated with n-BuLi (18.6 mL, 1.6 M in heptane, 29.7 mmol) over 3-4 minutes and stirred for 1.0 h at −78° C. The clear yellow solution was warmed to −17° C. over 0.25 h, recooled to −78° C. and charged with crude geraniol chloride (82% pure, 4.27 g, 24.75 mmol) in THF (20 mL) over 0.25 h. The reaction was warmed to room temperature overnight. HPLC analysis showed no remaining geraniol chloride.

Clear orange crude TMS-alkyne was cooled to 0° C. and charged with TBAF in THF (30-33 mL, 1 M in THF, 30-33 mmol) and stirred for 4 h at rt. Heptane was added (100 mL) and the cloudy pale brown solution treated with 10 wt % NaCl (100 mL) giving a substantial emulsion. The layers were separated, the aqueous layer washed with heptane (2×50 mL), and the organic layer washed twice with H₂O (100 mL) and the pH adjusted with HCl (˜2 mL, 12 M, 24 mmol) to pH=3 which turned both phases light yellow. The washed organics were dried with 20 wt % NaCl (100 mL) and Na₂SO₄ and concentrated to a brown oil. HPLC analysis showed incomplete loss of TMS and ¹H NMR confirmed the presence of TMS and a depression in alkyne signal area at 1.94 ppm. The crude oil was treated with TBAF in THF (100 mL, 1 M in THF, 100 mmol) and stirred for 6 h. HPLC confirmed no starting material. MTBE (300 mL) was added followed by heptane (100 mL) and the combined organics washed with H₂O (3×100 mL). Back extracted the aqueous layers with MTBE (2×100 mL) and the combined organics were washed with 10 wt % NaCl (100 mL), 20 wt % NaCl (200 mL) and dried over Na₂SO₄ before concentration to a yellow-orange oil. Flash chromatography (ISCO, CH₂Cl₂/Heptane) yielded 2.1 g of 16 a colorless oil (48.2%). HPLC>99% at 215 nm. ¹H NMR (CDCl₃, 400 MHz) δ 5.18 (br t, J=6.3 Hz, 1H), 5.10 (br t, J=6.8 Hz, 1H), 2.22 (m, 4H), 2.07 (m, 4H), 1.94 (t, J=2.5 Hz, 1H), 1.68 (s, 3H), 1.62 (s, 3H), 1.60 (s, 3H). ¹³C NMR (CDCl₃, 100 MHz) δ 136.9, 131.5, 124.4, 122.6, 84.7, 68.2, 39.8, 27.3, 26.8, 25.8, 19.1, 17.8, 16.3.

Synthesis of Vinyl Iodide (C3) For Use in Examples 8a-8c

In a toluene-rinsed, N₂-purged flask was added Cp₂ZrCl₂ (349.1 mg, 1.189 mmol) and AlMe₃ (11.9 mL, 2 M in toluene, 23.87 mmol) and the clear yellow solution cooled to 0° C. The stirred solution was treated with iso-butanol (220 μL) down the side of the flask over 1 minute (exothermic!) and stirred for 0.25 h. Alkyne (16, 2.1 g, 11.89 mmol) in toluene (3 mL) was added over 1 minute down side of flask via syringe and the alkyne source container rinsed with toluene (2×2 mL) and added to the reaction vessel. The solution was stirred at rt overnight giving a dark orange solution which showed incomplete reaction by HPLC. An additional 19 h stirring showed complete consumption of starting alkyne by HPLC. A solution of iodine (3.484 g, 13.36 mmol) in THF (10.75 mL) was prepared, the vinyl alane cooled to −30° C. and the I₂ solution added over 0.25 h giving a dark red-brown solution which was warmed to 0° C. and stirred for 0.5 h. IPA (5 mL) was added slowly (gas evolution, exotherm) followed by heptane (200 mL) and H₂O (10 mL) to give a suspension of white solids in a yellow solution. Celite (˜20 g) was added, stirred, filtered, and the solids rinsed with 2 volumes of heptane (˜20 mL each). The clear layers were separated, the organics washed with HCl (20 mL, 2.5 M), aqueous Na₂S₂O8 (20 mL), 20 wt % NaCl (20 mL) dried over Na₂SO₄ and concentrated. Toluene was removed by distillation on rotovap (30 mL heptane, 25° C., 17 mmHg) and the resulting oil subjected to flash chromatography on a base (Et₃N) treated SiO₂ column (ISCO, 0-10% CH₂Cl₂/heptane) returning 2.414 g (63.8%) of product as a clear, colorless oil. HPLC: <99% at 215 nm. ¹H NMR (CDCl₃, 400 MHz) δ 5.87 (dd, J=2.1, 1.0 Hz, 1H), 5.08 (m, 2H), 4.70 (d, J=9.8 Hz, 1H), 2.23 (m, 2H), 2.13 (m, 2H), 2.05 (m, 2H), 1.99 (m, 2H), 1.84 (s, 3H), 1.69 (s, 3H), 1.61 (s, 3H), 1.59 (s, 3H). ¹³C NMR (CDCl₃, 100 MHz) δ148.0, 136.2, 131.6, 124.4, 123.1, 74.9, 39.8, 39.7, 26.9, 26.4, 25.9, 24.1, 17.7, 16.2. gCOSY, gHSQCAD, gHMBCAD conform to expected.

### Example 8a: Synthesis of (R,E,E)-Alpha Tocotrienol Quinone

(R,E,E)-Alpha Tocotrienol Quinone was synthesized according to the following scheme.

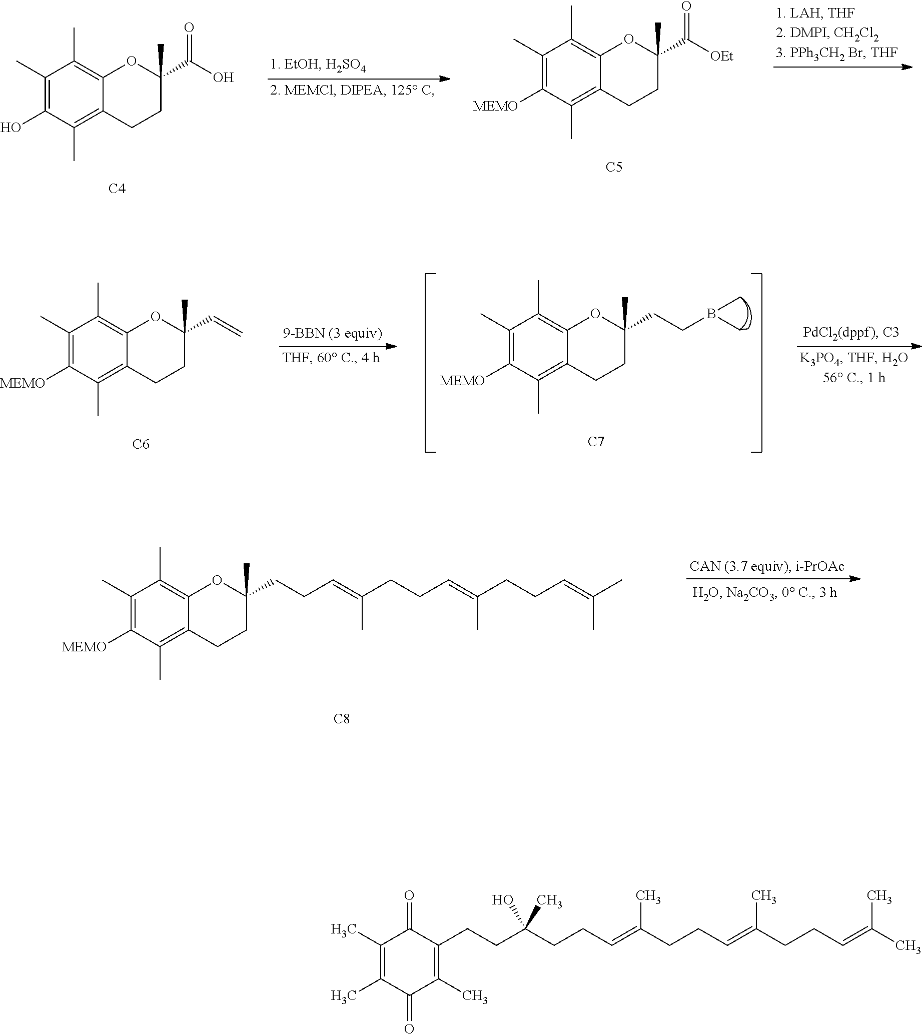

The phenolic oxygen of the ethyl ester of Trolox C4, which can be prepared using procedures known to one of ordinary skill in the art including those as described in WO 2016/100576, was protected as a MEM ether, the ether was removed with formic acid at elevated temperature to return chromanol. The MEM group was removed during the oxidation of C8 with CAN in i-PrOAc/H₂O, returning (R,E,E)-Alpha Tocotrienol Quinone.

### Synthesis of (S)-Ethyl Troloxate

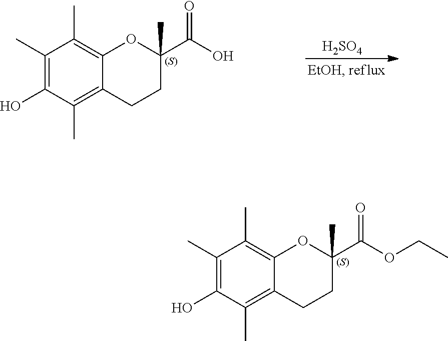

In a degassed 500 mL RB flask, (S)-Trolox (25 g, 100 mmol) was dissolved in ethanol (150 mL) and stirred. H₂SO₄ (conc., 5 mL) in ethanol (50 mL) was added and stirred at reflux (85° C.) overnight. The reaction mixture was concentrated in vacuo to remove ethanol then redissolved in ethyl acetate (100 mL). The mixture was then washed with DI H₂O (100 mL) and 20 wt % NaCl (100 mL), dried with sodium sulfate (1 g), filtered and concentrated in vacuo at no more than 50° C. Residual solvent was chased off with heptanes (3×3 mL) at 0 torr until constant mass which returned 25 g of pale brown solid (90%). HPLC: 94%, 215 nm. IR (ATR, neat) 3522.5, 2987.6, 2930.2, 1731.8, 1447.1, 1261.4, 1224.6, 1182.6, 1139.4, 1107.2, 1058.8, 1021.8, 947.7, 875.5, 830.2, 809.8, 767.2, 677.2 cm⁻.

### Synthesis of (S)-MEM Ethyl Troloxate

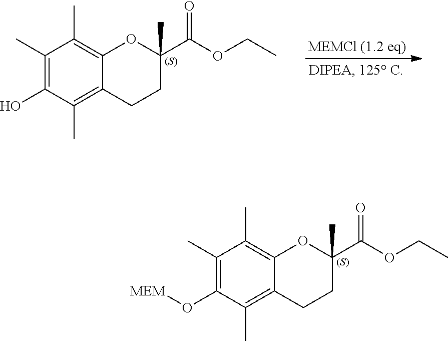

In a degassed 250 mL RB flask, ester (25 g, 90 mmol), MEMCl (15 mL, 135 mmol) and DIPEA (100 mL, 1.0M) were added and stirred for overnight at 125° C. The reaction mixture was then cooled to room temperature and diluted with THF (200 mL), washed with DI water (200 mL), 20 wt % NaCl (200 mL), dried with sodium sulfate (1 g), filtered and concentrated in vacuo at no more than 30° C. Residual solvent was chased off with heptanes (3×3 mL) at 0 torr until constant mass returning 30.0 g of brown/orange oil. Purification was performed by silica gel chromatography, eluting from 0-20% ethyl acetate in heptanes which returned 29.0 g of (S)-C5 clear yellow oil. The oil solidified to a yellow crystalline solid (88% yield) upon standing. HPLC: 98%, 255nm.¹H NMR (CDCl₃, 400 MHz) δ 4.93 (s, 2H), 4.11 (q, J=7.1 Hz, 2H), 3.94, (m, 2H), 3.60 (m, 2H), 3.40 (s, 3H), 2.60-2.40 (m, 3H), 2.18 (s, 3H), 2.15 (s, 3H), 2.10 (s, 3H), 1.86 (ddd, J=13.3, 10.6, 6.3 Hz, 1H), 1.59 (s, 3H), 1.17 (t, J=7.1 Hz, 3H). ¹³C NMR (CDCl₃, 100 MHz) δ 173.8, 148.2, 147.3, 128.3, 126.2, 123.1, 117.3, 98.5, 72.0, 69.3, 61.2, 59.2, 30.5, 25.5, 21.1, 14.2, 13.5, 12.6, 12.0. IR (ATR, neat) 2981.3, 2934.7, 1750.1, 1729.5, 1455.8, 1403.8, 1254.1, 1198.1, 1177.0, 1138.0, 1102.4, 1089.4, 1015.16, 942.8, 849.1, 764.0, 677.2 cm⁻¹.

### Synthesis of (S)-MEM Troloxol

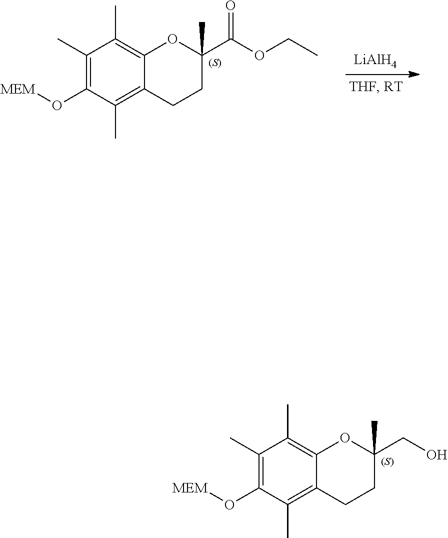

In a degassed 1 L RB flask, ester (25 g, 68 mmol) in THF (100 mL) was added to a well stirring suspension of LiAlH₄ (7.77 g, 205 mmol) in THF (400 mL) and stirred at room temperature for 0.5 h. The reaction mixture was slowly poured onto chilled aq. HC1 (50 mL, 2.5 M, 125 mmol) and DI water (400 mL). The product was the extracted with ethyl acetate (400 mL), dried with sodium sulfate (1 g), filtered and concentrated in vacuo at no more than 30° C. Residual solvent was chased off with heptanes (3×3 mL) at 0 torr until constant mass (15.0 g, 68% yield) returning an orange/yellow oil. HPLC: 96%, 215nm. ¹H NMR (CDCl₃, 400 MHz) δ 4.94, (s, 2H), 3.96 (m, 2H), 3.62-3.60 (m, 4H), 3.40 (s, 3H), 2.62 (m, 2H), 2.18 (s, 3H), 2.15 (s, 3H), 2.08 (s, 3H), 1.99 (ddd, J=13.6, 9.5, 7.4 Hz, 1H), 1.99 (s, 1H), 1.73 (ddd, J=13.6, 6.1, 4.9 Hz, 1H), 1.22 (s, 3H). ¹³C NMR (CDCl₃, 100 MHz) δ 147.5, 147.1, 128.4, 126.4, 123.0, 117.6, 98.5, 75.5, 72.0, 69.5, 69.3, 59.2, 27.7, 20.6, 20.3, 13.5, 12.6, 12.1.

### Synthesis of (S)-MEM Troloxal

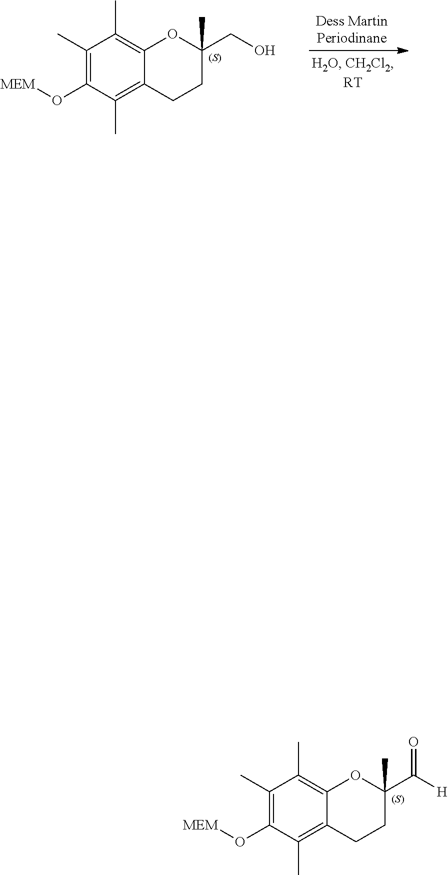

In a degassed 250 mL RB flask, alcohol (15 g, 46 mmol) was dissolved in methylene chloride (100 mL). Then Dess Martin Periodinane (DMP, 27.5 g, 65 mmol) was added to the mixture and stirred at room temperature for 0.5 h, while adding DI water 500 μL over 0.5 h. The reaction was then filtered and washed/rinsed with DI H₂O (100 mL) and 20 wt % NaCl solution (100 mL). The organic layer was then separated, dried with sodium sulfate (1 g), filtered and concentrated in vacuo at no more than 30° C. Residual solvent was chased off with heptanes (3×3 mL) at 0 torr until constant mass we obtained. About 20 g of aldehyde as a crude yellow oil was (16.2 g, 72% yield) used directly in the next step. ¹H NMR (CDCl₃, 400 MHz) δ 9.60 (S, 3H), 4.93 (S, 2H), 3.95 (m, 2H), 3.60 (m, 2H), 3.40 (S, 3H), 2.68-2.46 (m, 2H), 2.26 (m, 1H), 2.20 (s, 3H), 2.17 (s, 3H), 2.09 (s, 3H), 1.90 (m, 1H), 1.80 (m, 1H), 1.23 (s, 3H). ¹³C NMR (CDCl₃, 100 MHz) δ 205.0, 147.7, 128.7, 126.6, 123.2, 117.7, 98.5, 80.5, 71.9, 69.4, 59.2, 27.8, 21.6, 20.4, 13.5, 12.6, 12.1. IR (ATR, neat) 2980.6, 2932.7, 2826.6, 1736.5, 1454.8, 1402.5, 1375.1, 1253.5, 1105.2, 1088.6, 1061.7, 1007.2, 939.6, 807.4 cm⁻¹.

### Synthesis of (S)-MEM Troloxene

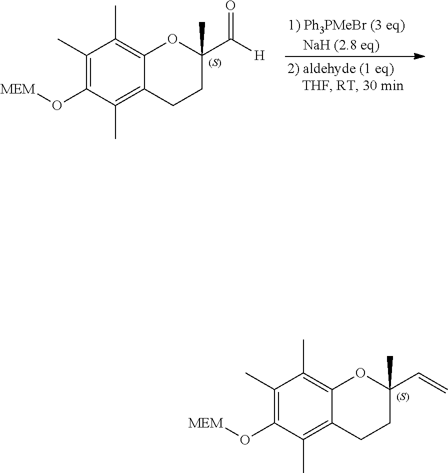

In a degassed 500 mL RB flask, methyltriphenylphosphonium bromide (51.0 g, 140 mmol) and NaH (5.24 g, 60% dispersion in mineral oil, 130 mmol) were suspended in THF (250 mL) at room temperature and stirred for 1 hr. Aldehyde (15 g, 46 mmol) was dissolved in THF (50 mL) and added to the reaction mixture, then stirred at room temperature for 1 h. The reaction was diluted with methylene chloride (200 mL) and the organic layer washed with aqueous sodium bicarbonate (200 mL, 1 M, 200 mmol) (slow addition), DI water (200 mL) and 20 wt % NaCl (200 mL) and dried with sodium sulfate (1 g), filtered and concentrated in vacuo at no more than 30° C. Residual solvent was chased off with heptanes (3×3 mL) at 0 torr until constant mass we obtained. A brown/yellow oil (55 g) was obtained which solidified upon storage at 2-8° C. Isolation was performed via a silica column, eluting from 0-15% ethyl acetate in heptanes returning a clear yellow oil of the desired product (7.0 g, 51% yield). HPLC: 86%, 255 nm. ¹H NMR (CDCl₃, 400 MHz) δ 5.85 (dd, J=17.3, 10.8 Hz, 1H), 5.13 (dd, J=17.3, 1.4 Hz, 1H), 5.03 (dd, J=10.8 Hz, 1.4 Hz, 1H), 4.94 (s, 2H), 3.96 (m, 2H), 3.61 (m, 2H), 3.40 (S, 3H), 2.60-2.48 (m, 2H), 2.219 (s, 3H), 2.14 (s, 3H), 2.12 (s, 3H), 1.91 (ddd, J =13.4, 6.1, 5.3 Hz, 1H), 1.82 (ddd, J=13.4 9.5, 6.2 Hz, 1H), 1.39 (s, 3H). ¹³C NMR (CDCl₃, 100 MHz) ϵ 148.2, 146.8, 141.9, 128.1, 126.1, 122.6, 117.7, 113.4, 98.5, 75.6, 72.0, 69.3, 59.2, 31.9, 27.2, 21.0, 13.5, 12.6, 12.0. IR (ATR, neat) 2977.2, 2930.4, 2826.3, 1453.8, 1403.0, 1370.8, 1253.8, 1087.9, 1061.4, 917.5, 843.6, 808.0, 743.4, 697.4 cm⁻¹.

### Synthesis of (R)-MEM-a-Tocotriene

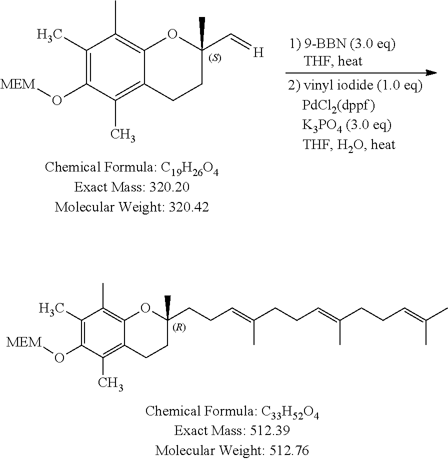

In a degassed 100 mL RB flask, troloxene (1.6 g, 5.0 mmol), was dissolved in THF (20 mL); then 9-BBN (30 mL, 0.5 M in THF, 15 mmol) was added and stirred at 60° C. for 1 h. PdCl₂(dppf)₂ (408 mg, 0.5 mmol) was added followed by the vinyl iodide C3 (1.61 g, 5.0 mmol) in THF (5 mL) and then K₃PO₄ (4.78 mL, 45% w/w in DI water) was added (slowly) and stirred at 60° C. for 1 h. The reaction was cooled to room temperature and then slowly quenched with hydrogen peroxide (10 mL, 35% w/w in DI water). The product was then extracted with heptanes (20 mL) and washed with DI water (20 mL), dried with sodium sulfate (500 mg), filtered and concentrated in vacuo at no more than 30° C. Residual solvent was chased off with heptanes (3×3 mL) at 0 torr until constant mass we obtained. The resulting brown oil (4.05 g) was purified by silica column, eluting from 0-40% ethyl acetate in heptanes returning a clear yellow oil of the desired product (1.57 g, 80%). HPLC: 81%, 215 nm. ^{H}H NMR (CDCl₃, 400 MHz) δ 5.10 (m, 3H), 4.94 (s, 2H), 3.96 (m, 2H), 3.61 (t, 2H), 3.40 (s, 3H), 2.58 (t, J=6.8 Hz, 2H), 2.18 (s, 3H), 2.14 (s, 3H), 2.13-2.05 (m, 10 H), 1.97 (m, 4H), 1.81 (ddd, J=20.2, 13.5, 6.6 Hz, 2H), 1.68 (s, 3H), 1.60-1.55 (m, 10H), 1.25 (s, 3H). ¹³C NMR (CDCl₃, 100 MHz) δ 148.2, 146.7, 135.2, 135.1, 131.4, 127.9, 125.9, 124.6, 124.5, 124.3, 123.1, 117.6, 98.5, 74.7, 72.0, 69.4, 59.2, 39.9, 39.8, 39.8, 31.5, 26.9, 26.8, 25.8, 24.0, 22.4, 20.8, 17.8, 16.2, 16.0 ,12.7, 11.9, 11.8. IR (ATR, neat) 2924.5, 2855.1, 1451.7, 1403.5, 1376.1, 1263.6, 1164.8, 1088.6, 1012.5 cm⁻¹.

### Synthesis of R-α-Tocotrienol Quinone

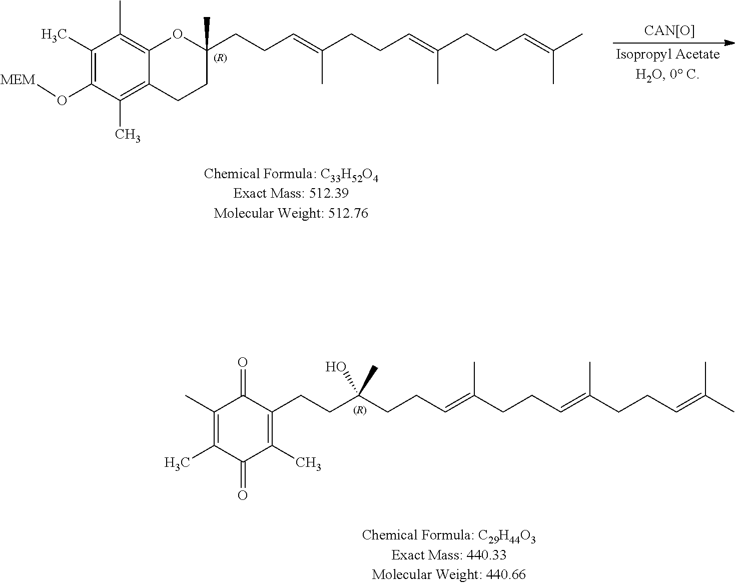

In a degassed 100mL RB flask, methyl R-α-tocotrienol (1.55 g, 3.0 mmol), isopropyl acetate (20 mL) and DI water (2 mL) were added at room temperature and stirred. The mixture was then cooled to 0-5° C. and stirred rapidly (900 RPM). In a separate vessel, cerium ammonium nitrate (CAN, 5.8 g, 10.5 mmol) was dissolved in DI water (8 mL) then sodium carbonate (960 mg) was added (slowly) and stirred for 15 min. After stirring, the CAN solution was added via a pressure-equalized dropping funnel into the reaction mixture over 10 min while maintaining an internal temperature of no more than 5° C. The suspension was then stirred for 3 h at 1500 RPM at room temperature.

After 3 h, the layers were split; layers separated in less than 10 sec. The top organic layer appeared clear with a green tint. The bottom aqueous layer appeared cloudy with a yellow tint. The organic phase from the reaction was then washed with 20 wt % NaCl (15 mL), dried with sodium sulfate (500 mg), filtered and concentrated in vacuo at no more than 30° C. Residual solvent was chased off with heptanes (3×3 mL) at 0 torr until constant mass (˜1.6 g) of an orange oil was obtained. Isolation was performed via a silica column, eluting from 0-25% ethyl acetate in heptanes returning 900 mg of R-EPI-743 as a clear orange oil (71%). HPLC: 86%, 255 nm. In comparison, the semisynthetic R-EPI-743 is also a clear orange oil.

### Example 8b: Synthesis of (Rac)-Alpha Tocotrienol Quinone

(rac)-Alpha Tocotrienol Quinone was synthesized according to the following scheme.

### Synthesis of Rac-Ethyl Toloxate

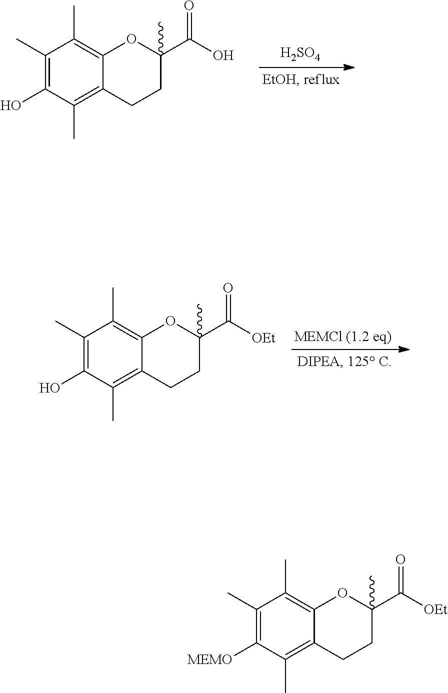

In a degassed 500 mL RB flask, rac-Trolox (25 g, 100 mmol) was dissolved in ethanol (150 mL) and stirred. H₂SO₄ (conc., 5 mL) in ethanol (50 mL) was added and stirred at reflux (85° C.) overnight. The reaction mixture was concentrated in vacuo to remove ethanol then redissolved in ethyl acetate (100 mL). The mixture was then washed with DI H₂O (100 mL) and 20 wt % NaCl (100 mL), dried with sodium sulfate (1 g), filtered and concentrated in vacuo at no more than 50° C. Residual solvent was chased off with heptanes (3×3 mL) at 0 torr until constant mass, giving 26.5 g of pale yellow/grey solid (99.6%). HPLC: 98%, 215 nm. IR (ATR, neat) 3522.5, 2987.6, 2930.2, 1731.8, 1447.1, 1261.4, 1224.6, 1182.6, 1139.4, 1107.2, 1058.8, 1021.8, 947.7, 875.5, 830.2, 809.8, 767.2, 677.2 cm⁻¹.

In a degassed 250 mL RB flask, ester (25 g, 90 mmol), MEMCl (22.4 mL, 24.7 g, 108 mmol) and DIPEA (198 mL, 0.5M) were added and stirred for overnight at 125° C. The reaction mixture was then cooled to room temperature and diluted with THF (300 mL), washed with DI water (250 mL), 20 wt % NaCl (250 mL), dried with sodium sulfate (1 g), filtered and concentrated in vacuo at no more than 30° C. Residual solvent was removed with heptanes (3×3 mL) at 0 torr until constant mass returning 31.6 g of brown/orange oil. Purification by silica gel chromatography, eluting from 0-20% ethyl acetate in heptanes, gave 24.2 g of a clear yellow oil (73%) which solidified to a yellow crystalline solid upon standing. HPLC: 98%, 255 nm. ¹H NMR (CDCl₃, 400 MHz) δ 4.93 (s, 2H), 4.11 (q, J=7.1 Hz, 2H), 3.94, (m, 2H), 3.60 (m, 2H), 3.40 (s, 3H), 2.60-2.40 (m, 3H), 2.18 (s, 3H), 2.15 (s, 3H), 2.10 (s, 3H), 1.86 (ddd, J=13.3, 10.6, 6.3 Hz, 1H), 1.59 (s, 3H), 1.17 (t, J=7.1 Hz, 3H). ¹³C NMR (CDCl₃, 100 MHz) δ 173.8, 148.2, 147.3, 128.3, 126.2, 123.1, 117.3, 98.5, 72.0, 69.3, 61.2, 59.2, 30.5, 25.5, 21.1, 14.2, 13.5, 12.6, 12.0. IR (ATR, neat) 2981.3, 2934.7, 1750.1, 1729.5, 1455.8, 1403.8, 1254.1, 1198.1, 1177.0, 1138.0, 1102.4, 1089.4, 1015.16, 942.8, 849.1, 764.0, 677.2 cm⁻¹.

### Synthesis of Rac-MEM Troloxene

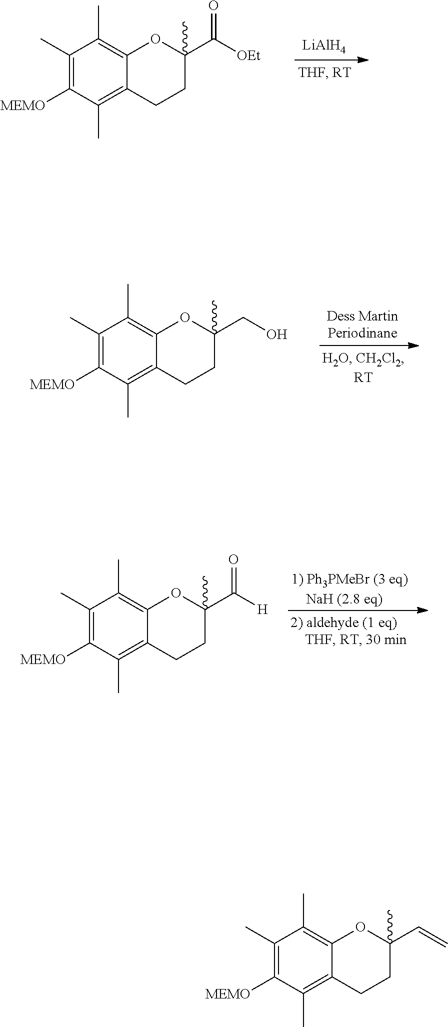

In a degassed 1 L RB flask, ester (24 g, 65.5 mmol) in THF (100 mL) was added to a well stirring suspension of LiAlH₄ (7.5 g, 196 mmol) in THF (400 mL) and stirred at room temperature for 0.5 h. The reaction mixture was slowly poured onto chilled aq. HCl (50 mL, 2.5 M, 125 mmol) and DI water (400 mL). The product was the extracted with ethyl acetate (400 mL), dried with sodium sulfate (1 g), filtered and concentrated in vacuo at no more than 30° C. Residual solvent was removed with heptanes (3×3 mL) at 0 torr until constant mass (18.0 g, 99.6% yield) returning the product as an orange/yellow oil. HPLC: 98%, 215 nm. ¹H NMR (CDCl₃, 400 MHz) δ 4.94, (s, 2H), 3.96 (m, 2H), 3.62-3.60 (m, 4H), 3.40 (s, 3H), 2.62 (m, 2H), 2.18 (s, 3H), 2.15 (s, 3H), 2.08 (s, 3H), 1.99 (ddd, J=13.6, 9.5, 7.4 Hz, 1H), 1.99 (s, 1H), 1.73 (ddd, J=13.6, 6.1, 4.9 Hz, 1H), 1.22 (s, 3H). ¹³C NMR (CDCl₃, 100 MHz) δ 147.5, 147.1, 128.4, 126.4, 123.0, 117.6, 98.5, 75.5, 72.0, 69.5, 69.3, 59.2, 27.7, 20.6, 20.3, 13.5, 12.6, 12.1.

In a degassed 250 mL RB flask, alcohol (23 g, 70 mmol) was dissolved in methylene chloride (150 mL). Then Dess Martin Periodinane (DMP, 42.13 g, 100 mmol) was added to the mixture and stirred at room temperature for 0.5 h, while adding DI water 500 μL over 0.5 h. The reaction was then filtered and washed/rinsed with DI H₂O (100 mL) and 20 wt % NaCl solution (100 mL). The organic layer was then separated, dried with sodium sulfate (1 g), filtered and concentrated in vacuo at no more than 30° C. Residual solvent was chased off with heptanes (3×3 mL) at 0 torr until constant mass was obtained. About 26.3 g of crude yellow oil was purified by silica column, eluting from 0-20% ethyl acetate in heptanes returning a yellow oil (16.2 g, 72% yield) the product used directly in the next step. ¹H NMR (CDCl ₃, 400 MHz) δ 9.61 (S, 3H), 4.94 (S, 2H), 3.95 (m, 2H), 3.60 (m, 2H), 3.40 (S, 3H), 2.68-2.46 (m, 2H), 2.26 (m, 1H), 2.20 (s, 3H), 2.17 (s, 3H), 2.09 (s, 3H), 1.90 (m, 1H), 1.80 (m, 1H), 1.23 (s, 3H). ¹³C NMR (CDCl₃, 100 MHz) δ 205.0, 147.7, 128.7, 126.6, 123.2, 117.7, 98.5, 80.5, 71.9, 69.4, 59.2, 27.8, 21.6, 20.4, 13.5, 12.6, 12.1. IR (ATR, neat) 2980.6, 2932.7, 2826.6, 1736.5, 1454.8, 1402.5, 1375.1, 1253.5, 1105.2, 1088.6, 1061.7, 1007.2, 939.6, 807.4 cm⁻¹.

In a degassed 500 mL RB flask, methyltriphenylphosphonium bromide (54.3 g, 150 mmol) and NaH (5.57 g, 60% dispersion in mineral oil, 140 mmol) were suspended in THF (200 mL) at room temperature and stirred for 1 hr. Aldehyde (16 g, 50 mmol) was dissolved in THF (50 mL) and added to the reaction mixture, then stirred at room temperature for 1 h. The reaction was diluted with methylene chloride (200 mL) and the organic layer washed with aqueous sodium bicarbonate (200 mL, 1 M, 200 mmol) (slow addition), DI water (200 mL) and 20 wt % NaCl (200 mL) and dried with sodium sulfate (1 g), filtered and concentrated in vacuo at no more than 30° C. Residual solvent was chased off with heptanes (3×3 mL) at 0 torr until constant mass. The brown/yellow oil (46 g) solidified upon storage at 2-8° C. Isolation was performed via a silica column, eluting from 0-15% ethyl acetate in heptanes returning the product as a clear yellow oil (14.2 g, 95% yield). HPLC: 98%, 255 nm. ¹H NMR (CDCl₃, 400 MHz) δ 5.85 (dd, J=17.3, 10.8 Hz, 1H), 5.10 (dd, J=17.3, 1.4 Hz, 1H), 5.01 (dd, J=10.8 Hz, 1.4 Hz, 1H), 4.94 (s, 2H), 3.96 (m, 2H), 3.61 (m, 2H), 3.4 (S, 3H), 2.60-2.48 (m, 2H), 2.219 (s, 3H), 2.14 (s, 3H), 2.12 (s, 3H), 1.91 (ddd, J=13.4, 6.1, 5.3 Hz, 1H), 1.82 (ddd, J=13.4 9.5, 6.2 Hz, 1H), 1.39 (s, 3H). ¹³C NMR (CDCl₃, 100 MHz) δ 148.2, 146.8, 141.9, 128.1, 126.1, 122.6, 117.7, 113.4, 98.5, 75.6, 72.0, 69.3, 59.2, 31.9, 27.2, 21.0, 13.5, 12.6, 12.0. IR (ATR, neat) 2977.2, 2930.4, 2826.3, 1453.8, 1403.0, 1370.8, 1253.8, 1087.9, 1061.4, 917.5, 843.6, 808.0, 743.4, 697.4 cm⁻¹.

### Synthesis of Rac-MEM-a-Tocotriene

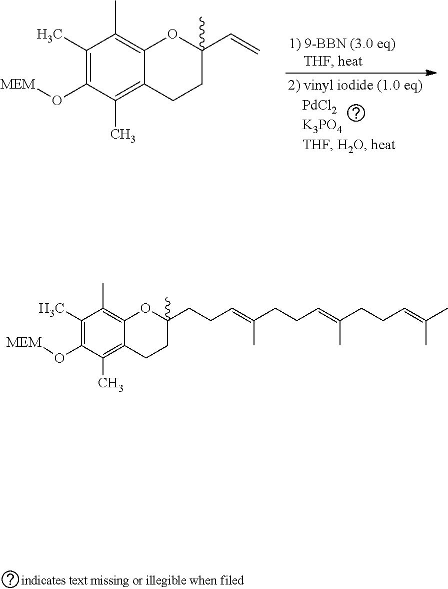

In a degassed 100 mL RB flask, troloxene (1.25 g, 5.0 mmol), was dissolved in THF (20 mL); then 9-BBN (30 mL, 0.5 M in THF, 15 mmol) was added and stirred at 60° C. for 1 h. PdCl₂(dppf₂) (408 mg, 0.5 mmol) was added followed by the vinyl iodide (C3, 1.61 g, 5.0 mmol) in THF (5 mL) and then K₃PO₄ (4.78 mL, 45% w/w in DI water) was added slowly and stirred at 60° C. for 1 h. The reaction was cooled to room temperature and then slowly quenched with hydrogen peroxide (10 mL, 35% w/w in DI water). The product was then extracted with heptanes (20 mL) and washed with DI water (20 mL), dried with sodium sulfate (500 mg), filtered and concentrated in vacuo at no more than 30° C. Residual solvent was chased off with heptanes (3×3 mL) at 0 torr until constant mass was obtained. The resulting brown oil (3.9 g) was purified by silica column, eluting from 0-40% ethyl acetate in heptanes returning a clear yellow oil (1.75 g, 80%). HPLC: 93%, 215 nm. ¹H NMR (CDCl₃, 400 MHz) δ 5.10 (m, 3H), 4.94 (s, 2H), 3.95 (m, 2H), 3.61 (t, 2H), 3.40 (s, 3H), 2.58 (t, J=6.8 Hz, 2H), 2.18 (s, 3H), 2.14 (s, 3H), 2.13-2.05 (m, 10 H), 1.99 (m, 4H), 1.81 (ddd, J=20.2, 13.5, 6.6 Hz, 2H), 1.68 (s, 3H), 1.64-1.53 (m, 10H), 1.25 (s, 3H). ¹³C NMR (CDCl₃, 100 MHz) δ 148.2, 146.7, 135.2, 135.1, 131.4, 127.9, 125.9, 124.6, 124.5, 124.3, 123.1, 117.6, 98.5, 74.7, 72.0, 69.4, 59.2, 39.9, 39.8, 39.8, 31.5, 26.9, 26.8, 25.8, 24.0, 22.4, 20.8, 17.8, 16.2, 16.0 ,12.7, 11.9, 11.8. IR (ATR, neat) 2924.5, 2855.1, 1451.7, 1403.5, 1376.1, 1263.6, 1164.8, 1088.6, 1012.5 cm⁻¹.

### Synthesis of Rac-α-Tocotrienol Quinone

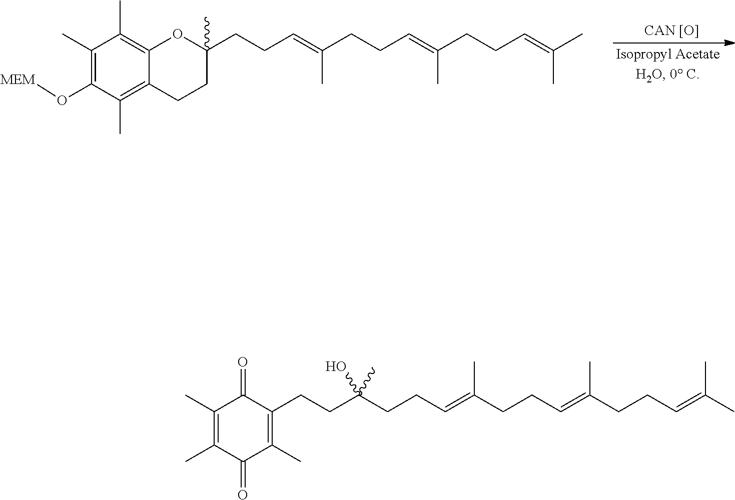

In a degassed 100mL RB flask, methyl rac-a-tocotrienol (1.75 g, 4.0 mmol), isopropyl acetate (20 mL) and DI water (2 mL) were added at room temperature and stirred. The mixture was then cooled to 0-5° C. and stirred rapidly (900 RPM). In a separate vessel, cerium ammonium nitrate (CAN, 8.77 g, 16.0 mmol) was dissolved in DI water (8 mL) then sodium carbonate (1.25g) was added (slowly) and stirred for 15 min. After stirring, the CAN solution was added via a pressure-equalized dropping funnel into the reaction mixture over 10 min while maintaining an internal temperature of no more than 5° C. The suspension was then stirred for 3 h at 1500 RPM at room temperature.

After 3 h, the layers were split; layers separated in less than 10 sec. The top organic layer appeared clear with a green tint. The bottom aqueous layer appeared cloudy with a yellow tint. The organic phase from the reaction was then washed with 20 wt % NaCl (15 mL), dried with sodium sulfate (500 mg), filtered and concentrated in vacuo at no more than 30° C. Residual solvent was chased off with heptanes (3×3 mL) at 0 torr until constant mass (˜1.5 g) of an orange oil was obtained. Isolation was performed via a silica column, eluting from 0-25% ethyl acetate in heptanes returning 1.25 g of rac-α-tocotrienol quinone as a clear orange oil (71%). HPLC: 93%, 255 nm. In comparison, the semisynthetic R-EPI-743 is also a clear orange oil.

### Example 8c: Synthesis of (S,E,E)-Alpha Tocotrienol Quinone

(S,E,E)-Alpha Tocotrienol Quinone was synthesized according to the following scheme.

### Synthesis of (R)-Ethyl Troloxate

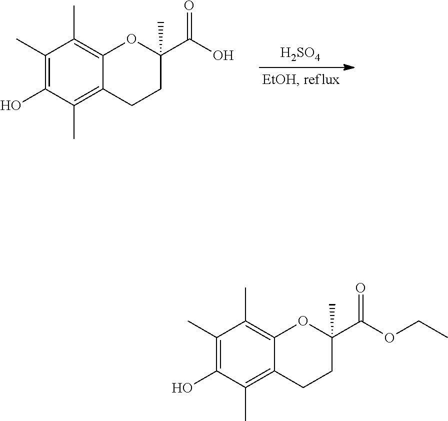 [SMILES: Cc1c(C)c2c(c(C)c1O)CC[C@](C)(C(=O)O)O2 ; "H2SO4" ; CCOC(=O)[C@@]1(C)CCc2c(C)c(O)c(C)c(C)c2O1]

In a degassed 500 mL RB flask, (R)-Trolox (25 g, 100 mmol) was dissolved in ethanol (150 mL) and stirred. H₂SO₄ (conc., 5 mL) in ethanol (50 mL) was added and stirred at reflux (85° C.) overnight. The reaction mixture was concentrated in vacuo to remove ethanol then redissolved in ethyl acetate (100 mL). The mixture was then washed with DI H₂O (100 mL) and 20 wt % NaCl (100 mL), dried with sodium sulfate (1 g), filtered and concentrated in vacuo at no more than 50° C. Residual solvent was chased off with heptanes (3×3 mL) at 0 torr until constant mass which returned 26 g of pale brown solid (94%). HPLC: 96%, 215 nm. IR (ATR, neat) 3525.1, 2987.6, 2930.2, 1715.9, 1447.1, 1261.4, 1224.6, 1182.6, 1139.4, 1107.2, 1058.8, 1021.8, 947.7, 875.5, 830.2, 809.8, 767.2, 677.2 cm⁻¹.

### Synthesis of (R)-MEM Ethyl Troloxate

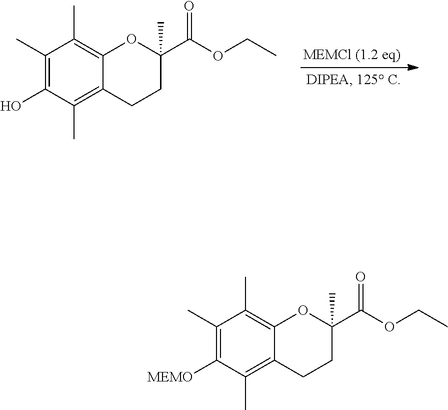

In a degassed 250 mL RB flask, starting ester (25 g, 90 mmol), MEMCl (15 mL, 135 mmol) and DIPEA (100 mL, 1.0M) were added and stirred for overnight at 125° C. The reaction mixture was then cooled to room temperature and diluted with THF (200 mL), washed with DI water (200 mL), 20 wt % NaCl (200 mL), dried with sodium sulfate (1 g), filtered and concentrated in vacuo at no more than 30° C. Residual solvent was chased off with heptanes (3×3 mL) at 0 torr until constant mass returning 33.8 g of brown/orange oil. Purification was performed by silica gel chromatography, eluting from 0-20% ethyl acetate in heptanes which returned 31.5 g of (R)-MEM ethyl troloxate as a clear yellow oil. The oil solidified to a yellow crystalline solid (95% yield) upon standing. HPLC: 98%, 255 nm. ¹H NMR (CDCl₃, 400 MHz) δ 4.93 (s, 2H), 4.11 (q, J=7.1 Hz, 2H), 3.94, (m, 2H), 3.60 (m, 2H), 3.40 (s, 3H), 2.60-2.40 (m, 3H), 2.18 (s, 3H), 2.15 (s, 3H), 2.10 (s, 3H), 1.86 (ddd, J=13.3, 10.6, 6.3 Hz, 1H), 1.59 (s, 3H), 1.17 (t, J=7.1 Hz, 3H). ¹³C NMR (CDCl₃, 100 MHz) δ 173.8, 148.2, 147.3, 128.3, 126.2, 123.1, 117.3, 98.5, 72.0, 69.3, 61.2, 59.2, 30.5, 25.5, 21.1, 14.2, 13.5, 12.6, 12.0. IR (ATR, neat) 2981.3, 2934.7, 1750.1, 1729.5, 1455.8, 1403.8, 1254.1, 1198.1, 1177.0, 1138.0, 1102.4, 1089.4, 1015.16, 942.8, 849.1, 764.0, 677.2 cm⁻¹.

### Synthesis of (R)-MEM Troloxol

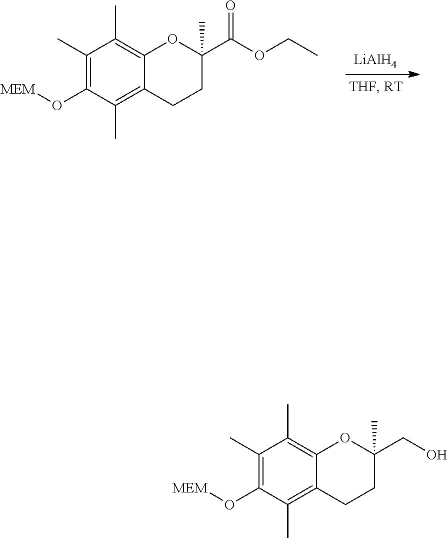

In a degassed 1 L RB flask, (R)-MEM ethyl troloxate (30 g, 82 mmol) in THF (100 mL) was added to a well stirring suspension of LiAlH₄ (9.32 g, 246 mmol) in THF (400 mL) and stirred at room temperature for 0.5 h. The reaction mixture was slowly poured onto chilled aq. HCl (50 mL, 2.5 M, 125 mmol) and DI water (400 mL). The product was the extracted with ethyl acetate (400 mL), dried with sodium sulfate (1 g), filtered and concentrated in vacuo at no more than 30° C. Residual solvent was chased off with heptanes (3×3 mL) at 0 torr until constant mass (25.0 g, 95% yield) returning (R)-MEM troloxol an orange/yellow oil. HPLC: 98%, 215 nm. ¹H NMR (CDCl₃, 400 MHz) δ 4.94, (s, 2H), 3.96 (m, 2H), 3.62-3.60 (m, 4H), 3.40 (s, 3H), 2.62 (m, 2H), 2.18 (s, 3H), 2.15 (s, 3H), 2.08 (s, 3H), 1.99 (ddd, J=13.6, 9.5, 7.4 Hz, 1H), 1.99 (s, 1H), 1.73 (ddd, J=13.6, 6.1, 4.9 Hz, 1H), 1.22 (s, 3H). ¹³C NMR (CDCl₃, 100 MHz) δ 147.5, 147.1, 128.4, 126.4, 123.0, 117.6, 98.5, 75.5, 72.0, 69.5, 69.3, 59.2, 27.7, 20.6, 20.3, 13.5, 12.6, 12.1.

### Synthesis of (R)-MEM Troloxal

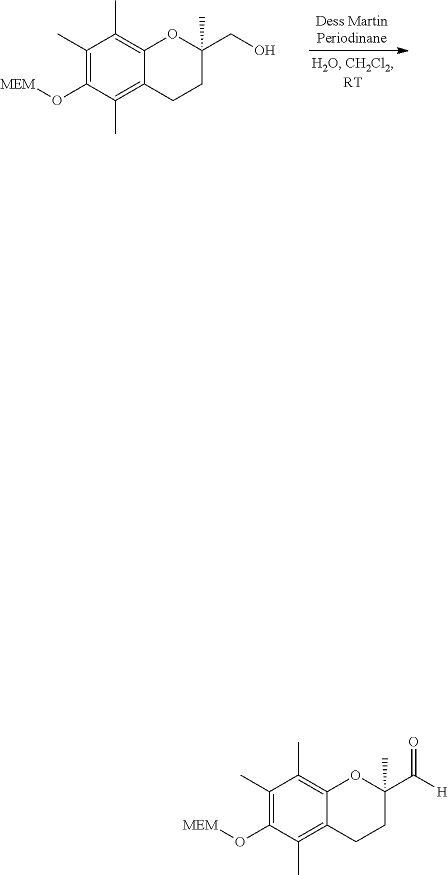

In a degassed 250 mL RB flask, (R)-MEM troloxol (25 g, 77 mmol) was dissolved in methylene chloride (150 mL). Then Dess Martin Periodinane (DMP, 45.8 g, 108 mmol) was added to the mixture and stirred at room temperature for 0.5 h, while adding DI water 500 μL over 0.5 h. The reaction was then filtered and washed/rinsed with DI H₂O (100 mL) and 20 wt % NaCl solution (100 mL). The organic layer was then separated, dried with sodium sulfate (1 g), filtered and concentrated in vacuo at no more than 30° C. Residual solvent was chased off with heptanes (3×3 mL) at 0 torr until constant mass we obtained. The resulting orange oil (40 g) was purified by a silica plug, eluting with 20% ethyl acetate in heptanes returning (R)-MEM troloxal as a clear yellow oil (18.2 g, 74%). ¹H NMR (CDCl₃, 400 MHz) δ 9.60 (S, 3H), 4.93 (S, 2H), 3.95 (m, 2H), 3.60 (m, 2H), 3.40 (S, 3H), 2.68-2.46 (m, 2H), 2.26 (m, 1H), 2.20 (s, 3H), 2.17 (s, 3H), 2.09 (s, 3H), 1.90 (m, 1H), 1.80 (m, 1H), 1.23 (s, 3H). ¹³C NMR (CDCl₃, 100 MHz) δ 205.0, 147.7, 128.7, 126.6, 123.2, 117.7, 98.5, 80.5, 71.9, 69.4, 59.2, 27.8, 21.6, 20.4, 13.5, 12.6, 12.1. IR (ATR, neat) 2980.6, 2932.7, 2826.6, 1736.5, 1454.8, 1402.5, 1375.1, 1253.5, 1105.2, 1088.6, 1061.7, 1007.2, 939.6, 807.4 cm⁻¹.

### Synthesis of (R)-MEM Troloxene

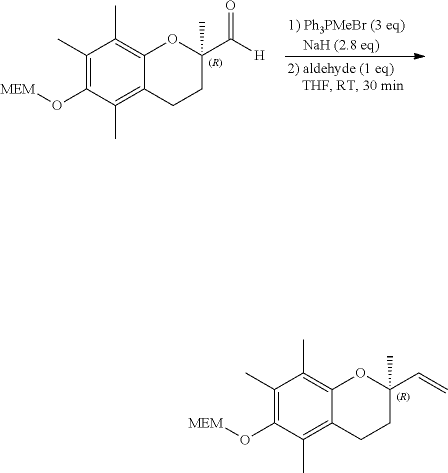

In a degassed 500 mL RB flask, methyltriphenylphosphonium bromide (60.0 g, 168 mmol) and NaH (6.30 g, 60% dispersion in mineral oil, 157 mmol) were suspended in THF (200 mL) at room temperature and stirred for 1 hr. (R)-MEM troloxal (18 g, 56 mmol) was dissolved in THF (50 mL) and added to the reaction mixture, then stirred at room temperature for 1 h. The reaction was diluted with methylene chloride (200 mL) and the organic layer washed with aqueous sodium bicarbonate (200 mL, 1 M, 200 mmol) (slow addition), DI water (200 mL) and 20 wt % NaCl (200 mL) and dried with sodium sulfate (1 g), filtered and concentrated in vacuo at no more than 30° C. Residual solvent was chased off with heptanes (3×3 mL) at 0 torr until constant mass we obtained. A brown/yellow oil (45 g) was obtained which solidified upon storage at 2-8° C. Isolation was performed via a silica column, eluting from 0-15% ethyl acetate in heptanes returning (R)-MEM troloxene as a clear yellow oil (13.5 g, 81% yield). HPLC: 98%, 215 nm. ¹H NMR (CDCl₃, 400 MHz) δ 5.85 (dd, J=17.3, 10.8 Hz, 1H), 5.13 (dd, J=17.3, 1.4 Hz, 1H), 5.03 (dd, J=10.8 Hz, 1.4 Hz, 1H), 4.94 (s, 2H), 3.96 (m, 2H), 3.61 (m, 2H), 3.40 (S, 3H), 2.60-2.48 (m, 2H), 2.219 (s, 3H), 2.14 (s, 3H), 2.12 (s, 3H), 1.91 (ddd, J=13.4, 6.1, 5.3 Hz, 1H), 1.82 (ddd, J=13.4 9.5, 6.2 Hz, 1H), 1.39 (s, 3H). ¹³C NMR (CDCl₃, 100 MHz) δ 148.2, 146.8, 141.9, 128.1, 126.1, 122.6, 117.7, 113.4, 98.5, 75.6, 72.0, 69.3, 59.2, 31.9, 27.2, 21.0, 13.5, 12.6, 12.0. IR (ATR, neat) 2977.2, 2930.4, 2826.3, 1453.8, 1403.0, 1370.8, 1253.8, 1087.9, 1061.4, 917.5, 843.6, 808.0, 743.4, 697.4 cm⁻¹.

### Synthesis of (S)-MEM-α-Tocotriene

In a degassed 100 mL RB flask, (R)-MEM troloxene (600 mg, 1.9 mmol), was dissolved in THF (10 mL); then 9-BBN (11.4 mL, 0.5 M in THF, 5.7 mmol) was added and stirred at 60° C. for 1 h. PdCl₂(dppf)₂ (155 mg, 0.19 mmol) was added followed by the vinyl iodide C3 (600 mg, 1.9 mmol) in THF (2 mL) and then K₃PO₄ (1.8 mL, 45% w/w in DI water) was added (slowly) and stirred at 60° C. for 1 h. The reaction was cooled to room temperature and then slowly quenched with hydrogen peroxide (5 mL, 35% w/w in DI water). The product was then extracted with heptanes (10 mL) and washed with DI water (10 mL), dried with sodium sulfate (500 mg), filtered and concentrated in vacuo at no more than 30° C. Residual solvent was chased off with heptanes (3×3 mL) at 0 torr until constant mass we obtained. The resulting brown oil (2.0 g) was purified by silica column, eluting from 0-40% ethyl acetate in heptanes returning (S)-MEM-α-tocotriene as a clear yellow oil of (520 mg, 54%). HPLC: 92%, 215 nm. ¹H NMR (CDCl₃, 400 MHz) δ 5.10 (m, 3H), 4.94 (s, 2H), 3.96 (m, 2H), 3.61 (t, 2H), 3.40 (s, 3H), 2.58 (t, J=6.8 Hz, 2H), 2.18 (s, 3H), 2.14 (s, 3H), 2.13-2.05 (m, 10 H), 1.97 (m, 4H), 1.81 (ddd, J=20.2, 13.5, 6.6 Hz, 2H), 1.68 (s, 3H), 1.60-1.55 (m, 10H), 1.25 (s, 3H). ¹³C NMR (CDCl₃, 100 MHz) δ 148.2, 146.7, 135.2, 135.1, 131.4, 127.9, 125.9, 124.6, 124.5, 124.3, 123.1, 117.6, 98.5, 74.7, 72.0, 69.4, 59.2, 39.9, 39.8, 39.8, 31.5, 26.9, 26.8, 25.8, 24.0, 22.4, 20.8, 17.8, 16.2, 16.0 ,12.7, 11.9, 11.8. IR (ATR, neat) 2924.5, 2855.1, 1451.7, 1403.5, 1376.1, 1263.6, 1164.8, 1088.6, 1012.5 cm⁻¹.

### Synthesis of S-α-Tocotrienol Quinone

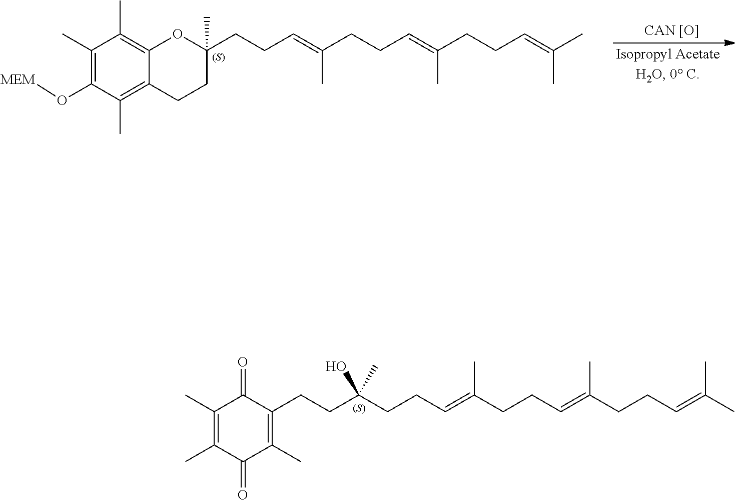

In a degassed 100 mL RB flask, (S)-MEM-α-tocotriene (500 mg, 1.0 mmol), isopropyl acetate (10 mL) and DI water (2 mL) were added at room temperature and stirred. The mixture was then cooled to 0-5° C. and stirred rapidly (900 RPM). In a separate vessel, cerium ammonium nitrate (CAN, 1.9 g, 3.5 mmol) was dissolved in DI water (8 mL) then sodium carbonate (300 mg) was added (slowly) and stirred for 15 min. After stirring, the CAN solution was added via a pressure-equalized dropping funnel into the reaction mixture over 10 min while maintaining an internal temperature of no more than 5° C. The suspension was then stirred for 3 h at 1500 RPM at room temperature.

After 3 h, the layers were split; layers separated in less than 10 sec. The top organic layer appeared clear with a green tint. The bottom aqueous layer appeared cloudy with a yellow tint. The organic phase from the reaction was then washed with 20 wt % NaCl (15 mL), dried with sodium sulfate (500 mg), filtered and concentrated in vacuo at no more than 30° C. Residual solvent was chased off with heptanes (3×3 mL) at 0 torr until constant mass (˜500 mg) of an orange oil was obtained. Isolation was performed via a silica column, eluting from 0-25% ethyl acetate in heptanes returning 350 mg of S-α-tocotrienol quinone as a clear orange oil 1 b (80%). HPLC: 96%, 255 nm. In comparison, the semisynthetic R-EPI-743 is also a clear orange oil.

### Example 9: Potency of Compounds in Q7 Striatal Cell Survival Assay Upon Cystine Deprivation

In neuronal cells excess extracellular glutamate inhibits the cystine/glutamate antiporter leading to intracellular cysteine depletion, GSH depletion, ROS production and cell death, a phenomenon termed oxidative glutamate toxicity or oxytosis. Q7 cells (ST HDH Q7/7; immortalized mouse striatal cells) challenged with cystine-free media recapitulate this phenotype. An initial screen was performed to identify compounds effective in rescuing Q7 cells from death resulting from cystine deprivation. This method is further described in Yonezawa et al., J. Neurochem 67, 566-573 (1996), and Li et al., J Neurosci., 23, 5816-5826 (2003).

DMEM (Catalog no. 11995-040), DMEM without Cystine (Catalog no. 21013-024), PBS (Phosphate buffered saline), Penicillin-streptomycin mix, L-Glutamine and Pyruvate were purchased from Gibco. Fetal Bovine Serum was obtained from Mediatech, Inc. Mouse striatum derived ST HDH Q7/7 (Q7) cells were obtained from Dr. M. MacDonald (Massachusetts General Hospital). Methionine and Vitamin K2 were purchased from Sigma Aldrich. Cell Titer Glo 2.0 was purchased from Promega. Geneticin (G418) Sulfate was purchased from Santa Cruz Biotechnology. Cell culture medium (Growth medium) was made by combining 50 mL Fetal Bovine Serum, 100 U/mL penicillin, 100 microgram/mL streptomycin and 400 microgram/mL Geneticin (G418) Sulfate; DMEM was added to make the volume up to 500 mL. Assay medium (without Cystine) was made by combining 50 mL Fetal Bovine Serum, 100 U/mL penicillin, 100 microgram/mL streptomycin, 4 mM L-Glutamine, 1 mM Pyruvate and 30 mg/L Methionine; DMEM without Cystine was added to make the volume up to 500 mL. During the course of the experiments, these solutions were stored at 4° C. The cells were grown in 10-cm diameter tissue culture-treated dishes. Every fourth day, the cells were subcultured by trypsinization and re-seeding at a cell density of 500,000 cells per dish.

Test samples were supplied in 1.5 mL glass vials. The compounds were diluted with an appropriate volume of DMSO to result in a 1 mM stock solution. Once dissolved, they were stored at −20 ° C.

Q7 cells were cultured routinely as described herein. For 384-well cell survival assays, cells were seeded in clear bottom, black wall 384-well tissue culture-treated polystyrene plates by resuspending a cell suspension at a density of 50,000 cells/mL in growth medium, then dispensing 60 microliters of cell suspension per well using either an electronic multichannel pipette or a Multidrop™ Combi Reagent Dispenser (ThermoFisher Scientific), corresponding to 3,000 cells/well. The cell-containing plates were incubated 5 hours at 33° C. in an atmosphere with 95% humidity and 5% CO₂ to allow attachment of the cells to the culture plate. 5 hours after cell seeding into the 384-well assay plate, the cell culture medium was replaced by washing 2 times with 70 microliters/well PBS (without Ca⁺⁺ and Mg⁺⁺) using a BioTek ELx405 plate washer. After the final aspiration, 60 microliters/well of assay medium (without cystine) was added using the Multidrop™ Combi Reagent Dispenser. Within 45 minutes, test compounds were then added to varying final concentrations using the Tecan D300e Digital Dispenser, with subsequent back-filling with DMSO diluent to a final concentration of 0.3% (v/v).

After medium change to cystine-free medium and compound addition, cell plates were incubated at 33° C. in an atmosphere of 5% CO₂ and 95% humidity. 18 hours later, the plates were equilibrated to room temperature for 15 minutes. Then, 10 microliters/well of room temperature Cell Titer Glo 2.0 reagent was added using the Multidrop™ Combi Reagent Dispenser. After 15 minutes of incubation at room temperature, the luminescence (100 ms integration time) per well was determined using the BioTek Synergy plate reader. Data was imported into Microsoft Excel. ACAS Curve Curator (John McNeil and Company) was then used to calculate the EC₅₀ values for each compound using standard four-parameter curve fitting algorithms.

The relative viability of test compound-treated cells were calculated relative to the average cystine-deprived, DMSO-treated cell viability (defined as 0% relative viability) and the average cystine-deprived, Vitamin K2 (1 micromolar) treated cell viability (defined as 100% relative viability). EC₅₀ was the concentration corresponding to 50% relative viability. The assay performance was gauged by Z-prime calculations on each assay plate, with observed Z-prime values of >0.5.

The Q7 cell survival EC₅₀ value of compounds of the present disclosure after 18 h was measured, and is shown in Table 1.

TABLE 1

| | EC₅₀ (nM) |
|---|---|
| | geometric mean |
| | (n ≥ 3 independent |
| Compound | experiments) |
| RRR-alpha-tocopherol | 175 |
| SRR-alpha-tocopherol | 372 |
| Equal mixture of all 8 | 313 |
| stereoisomers of | |
| alpha-tocopherol | |

The SRR isomer of alpha-tocopherol and all-racemic alpha tocopherol were approximately half as potent as the RRR isomer in rescuing Q7 cells from oxidative stress.

### Protocol for Example 10-12

DMEM (Catalog no. 11995-040), Penicillin-streptomycin mix, Geneticin (G418) Sulfate were purchased from Thermo Fisher Scientific. Fetal Bovine Serum was obtained from Sigma Aldrich. Mouse striatum-derived ST HDH Q7/7 (Q7) cells were obtained from Dr. M. MacDonald (Massachusetts General Hospital). HepG2 cells were obtained from ATCC. Normal human fibroblast cells (NHF) were obtained from Coriell. Cell culture medium for Q7 cell was prepared by combining 50 mL Fetal Bovine Serum, 100 U/mL penicillin, 100 microgram/mL streptomycin, and 400 microgram/mL Geneticin (G418) Sulfate; DMEM was added to bring the final volume to 500 mL. For HepG2 cells and NHF, cell culture medium was made by combining 50 mL Fetal Bovine Serum, 100 U/mL penicillin, and 100 microgram/mL streptomycin; DMEM was added to bring the final volume to 500 mL. During the course of the experiments, these solutions were stored at 4° C. The cells were grown in 150-cm² tissue culture-treated flasks until they reached 90% confluency.

Test samples were supplied in 1.5 mL glass vials. The compounds were diluted with an appropriate volume of DMSO to result in a 10 mM stock solution. Once dissolved, they were stored at −20 ° C.

Test samples were applied according to the following protocol:

Q7, HepG2, and NHF Cells were seeded separately in clear bottom, 96-well tissue culture-treated polystyrene plates by resuspending each cell suspension at a density of 200,000 cells/mL in cell culture medium, then dispensing 50 microliters of cell suspension per well using an electronic multichannel pipette, corresponding to 10,000 cells/well. The cell-containing plates were incubated 18 hours at 33° C. for Q7, 37° C. for HepG2 and NHF in an atmosphere with 95% humidity and 5% CO₂ to allow attachment of the cells to the culture plate.

Test compounds were prepared at two times the concentration in fresh cell media using 10 mM stock solution and adjust DMSO concentration to 0.6% (v/v) by adding DMSO. 18 hours after cell seeding, 50 microliters of culture medium containing test compounds were added to the wells in octuplicate.

After compound addition, cell plates were incubated at 33° C. or 37° C. in an atmosphere of 5% CO₂ and 95% humidity. 2, 6, and 24 hours later, 50 microliters/well of cell culture media was collected and dispensed into cluster tubes, then frozen on dry ice. Cells were washed once with 150 microliters/well of room temperature wash buffer, followed by aspiration of the buffer. After aspiration of the wash buffer, the cell plates were sealed with sealing foil, frozen on dry ice, then transferred to −80° C. freezer.

Quantification of compounds was determined by mass spectrometry.

### Example 10: Conversion of R,R,R-Alpha-Tocopherol and All-Rac Alpha-Tocopherol to the Corresponding Quinones in HepG2 Cells

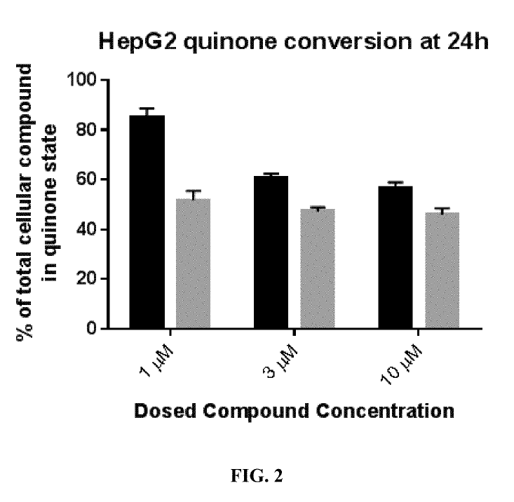

The rate of conversion of R,R,R-alpha-tocopherol (black bars) and a 1:1:1:1:1:1:1:1 equimolar mixture of all 8 stereoisomers of alpha-tocopherol (grey bars) to their corresponding quinones was measured in HepG2 cells and is shown in FIG. 2. The percent of total cellular compound in the quinone form was measured at three different dosed concentrations: 1 μM, 3 μM, and 10 μM. At all three concentrations, the percent of compound converted to the quinone form was higher when the specific R,R,R-alpha-tocopherol stereoisomer is dosed than when a mixture of all 8 stereoisomers of alpha-tocopherol was dosed.

Example 11: Conversion of R,R,R-Alpha-Tocopherol and All-Rac Alpha-Tocopherol to the Corresponding Quinones in NHF Cells

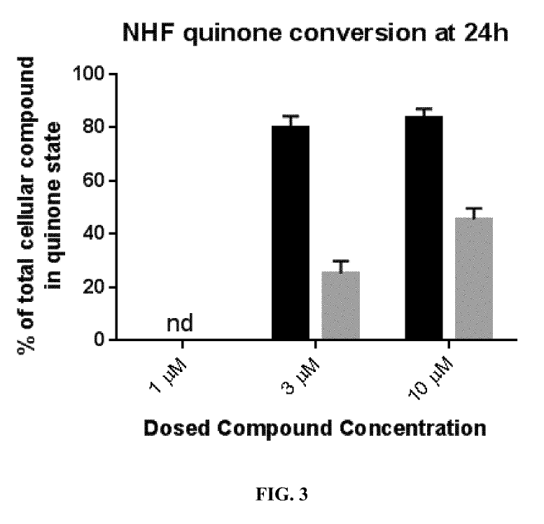

The rate of conversion of R,R,R-alpha-tocopherol (black bars) and a 1:1:1:1:1:1:1:1 equimolar mixture of all 8 stereoisomers of alpha-tocopherol (grey bars) to their corresponding quinones was measured in NHF cells and is shown in FIG. 3. The percent of total cellular compound in the quinone form was measured at two different dosed concentrations: 3 μM and 10 μM. At both concentrations, the percent of compound converted to the quinone form was higher when the specific R,R,R-alpha-tocopherol stereoisomer was dosed than when a mixture of all 8 stereoisomers of alpha-tocopherol was dosed. The percent of compound converted at a concentration of 3 μM was approximately 3 times higher with the specific RRR isomer than the mixture, and was approximately 2 times higher at a concentration of 10 μM.

Example 12: Conversion of R,R,R-Alpha-Tocopherol and All-Rac Alpha-Tocopherol to the Corresponding Quinones in Q7 Cells

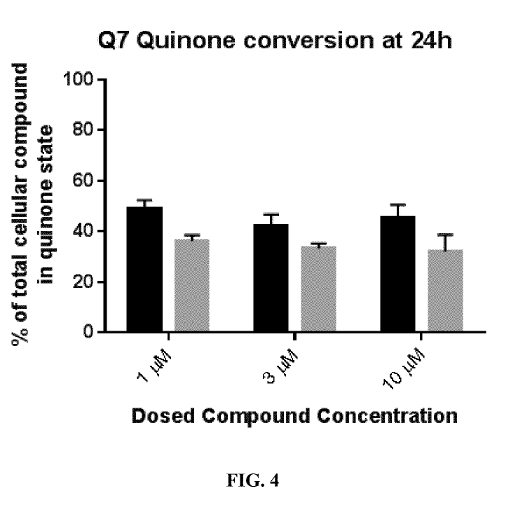

The rate of conversion of R,R,R-alpha-tocopherol (black bars) and a 1:1:1:1:1:1:1:1 equimolar mixture of all 8 stereoisomers of alpha-tocopherol (grey bars) to their corresponding quinones was measured in Q7 cells and is shown in FIG. 4. The percent of total cellular compound in the quinone form was measured at three different dosed concentrations: 1 μM, 3 μM, and 10 μM. At all three concentrations, the percent of compound converted to the quinone form was higher when the specific R,R,R-alpha-tocopherol stereoisomer was dosed than when a mixture of all 8 stereoisomers of alpha-tocopherol was dosed.

### Example 13: SQR Activity of Alpha-Tocopherol Quinones

SQR activity in the presence of varying concentrations of the isomers of quinone derivatives of Vitamin E was evaluated by monitoring the rate of quinone reduction using the absorbance of the quinone at 266 nm. Assays were conducted at 25° C. and pH 7.5 in a volume of 100 μL in the following buffer system: 100 mM Tris, 0.5 mM EDTA, 1.0% (w/v) Triton X-100 reduced. Sulfide (as Na₂S nonahydrate) as reducing substrate and sulfite (as Na₂SO₃) as sulfur acceptor substrate were held constant at saturating concentrations of 200 μM and 2 mM, respectively. Quinone substrates were added to final concentrations between 0-200 μM from DMSO stock solutions resulting in 2% final concentration of DMSO (enzymatic rates were independent of DMSO concentration in this range). SQR was added at 25 nM.

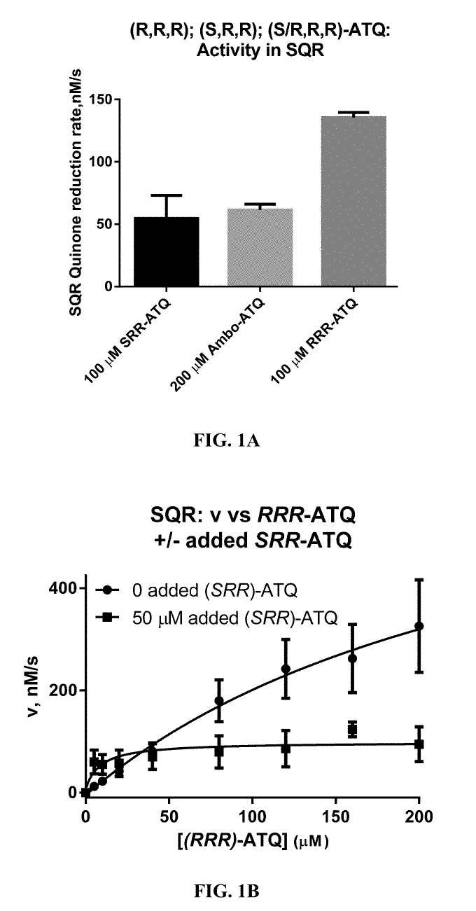

The rate of SQR reduction in the presence of 100 μM R,R,R-alpha-tocopherol quinone, 100 μM S,R,R-alpha-tocopherol quinone, and 200 μM of a 1:1 mixture of R,R,R-alpha-tocopherol quinone and S,R,R-alpha-tocopherol quinone (“Ambo-ATQ”) is shown in FIG. 1A. The reduction rate in the presence of 100 μM S,R,R-alpha-tocopherol quinone was about 50 nM/s, as was the 200 μM of a 1:1 ratio mixture of R,R,R-alpha-tocopherol quinone : S,R,R-alpha-tocopherol quinone. The reduction rate in the presence of 100 μM R,R,R-alpha-tocopherol quinone was more than twice the rate of the corresponding S,R,R stereoisomer, at around 125 nM/s. Notably, the 1:1 mixture, which contained the same amount of the R,R,R isomer as the pure R,R,R isomer, resulted in a much lower SQR activity than was seen with the pure R,R,R isomer, indicating that the S,R,R isomer has an inhibitory effect on the activity of the R,R,R isomer, resulting in lower SQR activity.

The rate of SQR reduction as a function of concentration is shown in FIG. 1B. In the absence of the S,R,R isomer, the rate of SQR reduction increases with increasing R,R,R-alpha-tocopherol quinone concentration. However, in the presence of S,R,R-alpha-tocopherol quinone, the rate of SQR reduction did not rise above about 100 nM/s, even with increasing concentration of the R,R,R isomer. Accordingly, this indicates the inhibitory effect of the S,R,R isomer on the activity of the R,R,R isomer, resulting in lower SQR activity.

### Example 14: Ointment Formulation

A composition of the present disclosure may be formulated as an ointment for topical administration with the composition listed in Table 2.

TABLE 2
Ointment formulation

| | Quantity |
|---|---|
| Ingredient | (mol %) |
| R,R,R-alpha-tocopherol | 10 |
| White wax | 5 |
| White petroleum | 85 |

### Example 15: Cream Formulation

A composition of the present disclosure may be formulated as a cream for topical administration with the composition listed in Table 3.

TABLE 3
Cream formulation

| | Quantity |
|---|---|
| Ingredient | (weight %) |
| Almond oil | 35 |
| White wax | 20 |
| R,R,R-alpha-tocopherol | 10 |
| Water | 45 |

While preferred embodiments of the present disclosure have been shown and described herein, it will be apparent to those skilled in the art that such embodiments are provided by way of example only. It is not intended that this disclosure be limited by the specific examples provided within the specification. While inventive subject matter has been described with reference to the aforementioned specification, the descriptions and illustrations of the embodiments herein are not meant to be construed in a limiting sense. Numerous variations, changes, and substitutions will now occur to those skilled in the art without departing from this disclosure. Furthermore, it shall be understood that all aspects of this disclosure are not limited to the specific depictions, configurations, or relative proportions set forth herein which depend upon a variety of conditions and variables. It should be understood that various alternatives to the embodiments described herein may be employed in practicing the inventive subject matter. It is therefore contemplated that this disclosure shall also cover any such alternatives, modifications, variations or equivalents. It is intended that the following claims define the scope of the inventive subject matter and that methods and structures within the scope of these claims and their equivalents be covered thereby.

What is claimed is:

1. A composition comprising a synthetic, non-plant based, substantially pure stereoisomer of an alpha, beta, gamma, or delta tocopherol or tocotrienol, or the quinone or hydroquinone form thereof, or a metabolite thereof.

2. The composition of claim 1, wherein the stereoisomer is (R,R,R)-alpha-tocopherol.

3. The composition of claim 1, wherein the stereoisomer is (R,E,E)-alpha-tocotrienol.

4. The composition of claim 1, wherein the stereoisomer is (R,R,R)-alpha-tocopherol quinone.

5. The composition of claim 1, wherein the stereoisomer is (R,E,E)-alpha-tocotrienol quinone.

6. The composition of claim 1, wherein the stereoisomer is (R,R,R)-alpha-tocopherol hydroquinone.

7. The composition of claim 1, wherein the stereoisomer is (R,E,E)-alpha-tocotrienol hydroquinone.

8. The composition of claim 1, wherein the substantially pure stereoisomer comprises at least about 95%.

9. A composition comprising one or more synthetic, non-plant based, substantially pure stereoisomers of:
(a) a tocopherol selected from the group consisting of: alpha-tocopherol, beta-tocopherol, gamma-tocopherol, and delta-tocopherol, wherein the one or more stereoisomers of the tocopherol comprise an asymmetric carbon at the 2-position of the chromanol ring, wherein the 2-position asymmetric carbon has an R configuration; or a metabolite thereof; or
(b) a tocopherol quinone selected from the group consisting of alpha-tocopherol quinone, beta-tocopherol quinone, gamma-tocopherol quinone, and delta-tocopherol quinone, wherein the one or more stereoisomers of the tocopherol quinone comprise an asymmetric carbon at the 3-position of the tail group, wherein the 3-position of the tail group has an R configuration; or a metabolite thereof; or
(c) a tocopherol hydroquinone selected from the group consisting of alpha-tocopherol hydroquinone, beta-tocopherol hydroquinone, gamma-tocopherol hydroquinone, and delta-tocopherol hydroquinone, wherein the one or more stereoisomers of the tocopherol hydroquinone comprise an asymmetric carbon at the 3-position of the tail group, wherein the 3-position of the tail group has an R configuration; or a metabolite thereof.

10. The composition of claim 9, wherein the one or more stereoisomers are alpha-tocopherol stereoisomers.

11. The composition of claim 9, wherein the one or more stereoisomers are alpha-tocopherol quinone stereoisomers.

12. The composition of claim 9, wherein the one or more stereoisomers are alpha-tocopherol hydroquinone stereoisomers.

13. The composition of claim 9, wherein the substantially pure stereoisomer is at least about 95%.

14. The composition of claim 1, wherein the composition is formulated for human use.

15. The composition of claim 1, wherein the composition is formulated for animal use.

16. A method of treating an individual, comprising: administering to a subject in need thereof a therapeutically effective amount or a physiologically effective amount of a composition according to claim 1.

17. A method for converting a tocopherol quinone or tocotrienol quinone to its corresponding chroman, comprising mixing the quinone with HCOOH and Pd/C.

18. The method of claim 17, wherein the mixture is heated.

19. A method for synthesizing (R,E,E)-α-tocotrienol quinone or (S,E,E)-α-tocotrienol quinone comprising
(a) treating the compound of Formula IXa or (IXb): 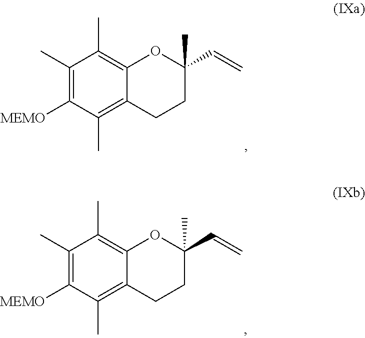
first with an organoborane in a first polar aprotic solvent followed by a palladium catalyst, a compound of Formula V: 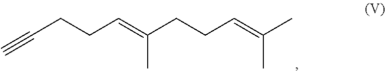
and a phosphate base in a second polar aprotic solvent and water to form a compound of Formula Xa or Xb, respectively: 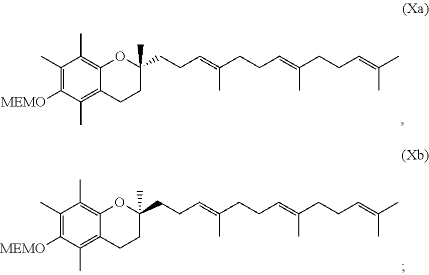
and
(b) treating the compound of Formula Xa or Xb with an oxidizing agent and a carbonate base in a mixture of a third polar aprotic solvent and water, to form (R,E,E)-α-tocotrienol quinone or (S,E,E)-α-tocotrienol quinone, respectively.

20. The method of claim 19, wherein the organoborane is 9-BBN, the palladium catalyst is PdCl₂(dppf), the phosphate base is potassium phosphate.

21. The method of claim 19, wherein the first polar aprotic solvent is selected from the group consisting of DMF and THF, the second polar aprotic solvent is selected from the group consisting of DMF and THF, and the third polar aprotic solvent is selected from the group consisting of isopropyl acetate.
